(19) United States
(12) Patent Application Publication
(10) Pub. No.: US 2023/0148575 A1
(43) Pub. Date: May 18, 2023

(54) GENETICALLY MODIFIED NON-HUMAN ANIMAL WITH HUMAN OR CHIMERIC IL1B AND/OR IL1A

(71) Applicants: Biocytogen Pharmaceuticals (Beijing) Co., Ltd., Beijing (CN); Biocytogen JiangSu Co., Ltd., Nantong, Jiangsu (CN)

(72) Inventors: Yuelei Shen, Beijing (CN); Meiling Zhang, Beijing (CN); Rui Huang, Beijing (CN); Yang Bai, Beijing (CN); Chaoshe Guo, Beijing (CN); Xiaofei Zhou, Beijing (CN)

(21) Appl. No.: 17/907,591

(22) PCT Filed: Apr. 7, 2021
(86) PCT No.: PCT/CN2021/085838

§ 371 Date: Sep. 28, 2022

(30) Foreign Application Priority Data
Apr. 7, 2020 (CN) 202010268583.1

(51) Int. Cl. A01K 67/027; C12N 15/11; C07K 16/24; G01N 33/50

(52) U.S. Cl. CPC A01K 67/0278; C12N 15/11; C07K 16/245; G01N 33/5088; A01K 2227/105; A01K 2217/072; A01K 2207/15; A01K 2267/03; G01N 2333/545

(57) ABSTRACT

The present disclosure relates to genetically modified non-human animals that express a human or chimeric (e.g., humanized) IL1B and/or IL1A, and methods of use thereof.

### CLAIM OF PRIORITY

This application claims the benefit of Chinese Patent Application App. No. CN202010268583.1, filed on Apr. 7, 2020. The entire contents of the foregoing application are incorporated herein by reference.

### TECHNICAL FIELD

This disclosure relates to genetically modified animal expressing human or chimeric (e.g., humanized) IL1B and/or IL1A, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory pathways typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an animal model with human IL1B or chimeric IL1B. The animal model can express human IL1B or chimeric IL1B (e.g., humanized IL1B) protein in its body. It can be used in the studies on the function of IL1B gene, and can be used in the screening and evaluation of anti-human IL1B antibodies. This disclosure is also related to an animal model with human IL1A or chimeric IL1A. The animal model can express human IL1A or chimeric IL1A (e.g., humanized IL1A) protein in its body. It can be used in the studies on the function of IL1A gene, and can be used in the screening and evaluation of anti-human IL1A antibodies. In some embodiments, the disclosure is related to IL1A/IL1B double gene humanized mice.

In addition, the animal models prepared by the methods described herein can be used in drug screening, pharmacodynamics studies, treatments for immune-related diseases (e.g., autoimmune disorders), and cancer therapy for human IL1B and/or IL1A target sites; they can also be used to facilitate the development and design of new drugs, and save time and cost. In summary, this disclosure provides a powerful tool for studying the function of IL1B and/or IL1A protein and a platform for screening drugs, e.g., antibodies, against autoimmune disorders (e.g., psoriasis).

In one aspect, the disclosure is related to a genetically-modified, non-human animal whose genome comprises at least one chromosome comprising a sequence encoding a human or chimeric interleukin 1 beta (IL1B).

In some embodiments, the sequence encoding the human or chimeric IL1B is operably linked to an endogenous regulatory element at the endogenous IL1B gene locus in the at least one chromosome. In some embodiments, the sequence encoding a human or chimeric IL1B is operably linked to an endogenous 5′ untranslated region (5′-UTR).

In some embodiments, the sequence encoding a human or chimeric IL1B comprises a sequence encoding an amino acid sequence that is at least 70%, 75%, 80%, 85%, 90%, 95%, 99%, or 100% identical to human IL1B (SEQ ID NO: 4).

In some embodiments, the sequence comprises a sequence that is at least 70%, 75%, 80%, 85%, 90%, 95%, 99%, or 100% identical to SEQ ID NO: 15, 16, 17, or 18.

In some embodiments, the animal is a mammal, e.g., a monkey, a rodent, or a mouse. In some embodiments, the mammal is a mouse.

In some embodiments, the animal does not express endogenous IL1B.

In some embodiments, the animal has one or more cells expressing human or chimeric IL1B.

In some embodiments, the expressed human or chimeric IL1B can bind to human IL-1 receptor type I (IL1R1). In some embodiments, the expressed human or chimeric IL1B can bind to endogenous IL1R1.

In one aspect, the disclosure is related to a genetically-modified, non-human animal. In some embodiments, the genome of the animal comprises a replacement of a sequence encoding a region of endogenous IL1B with a sequence encoding a corresponding region of human IL1B at an endogenous IL1B gene locus.

In some embodiments, the sequence encoding the corresponding region of human IL1B is operably linked to an endogenous regulatory element at the endogenous IL1B locus.

In some embodiments, the animal does not express endogenous IL1B, and the animal has one or more cells expressing human or chimeric IL1B.

In some embodiments, the replaced sequence encoding a region of endogenous IL1B comprises exon 1, exon 2, exon 3, exon 4, exon 5, exon 6, and/or exon 7, or a part thereof, of endogenous IL1B gene. In some embodiments, the animal is a rodent. In some embodiments, the animal is a mouse.

In some embodiments, the animal is a mouse, and the replaced sequence starts within exon 2 and ends within exon 7 of endogenous mouse IL1B gene.

In some embodiments, the animal is heterozygous with respect to the replacement at the endogenous IL1B gene locus. In some embodiments, the animal is homozygous with respect to the replacement at the endogenous IL1B gene locus.

In one aspect, the disclosure is related to a method for making a genetically-modified, non-human animal, comprising: replacing in at least one cell of the animal, at an endogenous IL1B gene locus, a sequence encoding a region of an endogenous IL1B with a sequence encoding a corresponding region of human IL1B.

In some embodiments, the sequence encoding the corresponding region of human IL1B comprises exon 2, exon 3, exon 4, exon 5, exon 6, and/or exon 7, or a part thereof, of a human IL1B gene.

In some embodiments, the sequence encoding the corresponding region of human IL1B encodes an amino acid sequence that is at least 70%, 75%, 80%, 85%, 90%, 95%, 99%, or 100% identical to SEQ ID NO: 4.

In some embodiments, the endogenous IL1B locus comprises exon 1, exon 2, exon 3, exon 4, exon 5, exon 6, and/or exon 7, or a part thereof, of the endogenous IL1B gene. In some embodiments, the animal is a rodent. In some embodiments, the animal is a mouse. In some embodiments, the animal is a mouse, and the replaced sequence starts from within exon 2 and ends within exon 7 of endogenous mouse IL1B gene.

In one aspect, the disclosure is related to a non-human animal comprising at least one cell comprising a nucleotide sequence encoding an exogenous IL1B polypeptide. In some embodiments, the exogenous IL1B polypeptide comprises at least 50 contiguous amino acid residues that are identical to the corresponding contiguous amino acid sequence of a human IL1B. In some embodiments, the animal expresses the exogenous IL1B.

In some embodiments, the exogenous IL1B polypeptide comprises an amino acid sequence that is at least 90%, 95%, or 99% identical to SEQ ID NO: 4.

In some embodiments, the nucleotide sequence is operably linked to an endogenous IL1B regulatory element of the animal.

In some embodiments, the nucleotide sequence is integrated to an endogenous IL1B gene locus of the animal.

In some embodiments, the animal in its genome comprises, preferably from 5′ to 3′: a mouse 5′ UTR, a sequence encoding the exogenous IL1B polypeptide, and a mouse 3′ UTR.

In one aspect, the disclosure is related to a method of making a genetically-modified non-human animal cell that expresses a chimeric IL1B, the method comprising: replacing at an endogenous IL1B gene locus, a nucleotide sequence encoding a region of endogenous IL1B with a nucleotide sequence encoding a corresponding region of human IL1B, thereby generating a genetically-modified non-human animal cell that includes a nucleotide sequence that encodes the chimeric IL1B. In some embodiments, the non-human animal cell expresses the chimeric IL1B. In some embodiments, the animal is a rodent. In some embodiments, the animal is a mouse.

In some embodiments, the nucleotide sequence encoding the chimeric IL1B is operably linked to an endogenous IL1B regulatory region, e.g., promoter.

In some embodiments, the animal as described herein further comprises a sequence encoding an additional human or chimeric protein. In some embodiments, the additional human or chimeric protein is interleukin 1 alpha (IL1A), IL-1 receptor type I (IL1R1), interleukin-1 receptor accessory protein (IL1RAP), programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40). In some embodiments, the additional human or chimeric protein is IL1A and the animal expresses the human or chimeric IL1A.

In some embodiments, the animal or mouse as described herein further comprises a sequence encoding an additional human or chimeric protein. In some embodiments, the additional human or chimeric protein is IL1A, IL1R1, IL1RAP, PD-1, CTLA-4, LAG-3, IL15 receptor, BTLA, PD-L1, CD3, CD27, CD28, CD47, CD137, CD154, TIGIT, TIM-3, GITR, SIRPα or OX40. In some embodiments, the additional human or chimeric protein is IL1A and the animal expresses the human or chimeric IL1A.

In one aspect, the disclosure is related to a method of determining effectiveness of an anti-IL1B antibody for treating an allergic disorder, comprising: a) administering the anti-IL1B antibody to the animal as described herein, in some embodiments, the animal has the allergic disorder; and b) determining effects of the anti-IL1B antibody in treating the allergic disorder.

In some embodiments, the allergic disorder is allergy, asthma, and/or atopic dermatitis.

In one aspect, the disclosure is related to a method of determining effectiveness of an anti-IL1B antibody for reducing an inflammation, comprising: a) administering the anti-IL1B antibody to the animal as described herein, in some embodiments, the animal has the inflammation; and b) determining effects of the anti-IL1B antibody for reducing the inflammation.

In one aspect, the disclosure is related to a method of determining effectiveness of an agent for treating an autoimmune disorder, comprising: a) administering the agent to the animal as described herein, in some embodiments, the animal has the autoimmune disorder; and b) determining effects of the agent for treating the autoimmune disorder. In some embodiments, the autoimmune disorder is rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

In some embodiments, the autoimmune disorder is psoriasis.

In some embodiments, the animal is a mouse and the psoriasis is induced by treating the mouse with imiquimod (IMQ). In some embodiments, the agent is a corticosteroid (e.g., dexamethasone). In some embodiments, the agent is an anti-IL1B antibody. In some embodiments, the anti-IL1B antibody is Gevokizumab or Canakinumab. In some embodiments, the effects are evaluated by clinical scores (e.g., Psoriasis Area Severity Index) and/or hematoxylin and eosin (HE) staining.

In one aspect, the disclosure is related to a method of determining effectiveness of an agent for treating an autoinflammatory disease, comprising: a) administering the agent to the animal as described herein, in some embodiments, the animal has the autoinflammatory disease; and b) determining effects of the agent for treating the autoinflammatory disease.

In some embodiments, the autoinflammatory disease is tumor necrosis factor receptor associated periodic syndrome (TRAPS), hyperimmunoglobulin D syndrome (HIDS)/mevalonate kinase deficiency (MKD), familial mediterranean fever (FMF), Still's disease, adult-onset Still's disease (AOSD), autoinflammatory periodic fever syndromes, cryopyrin-associated periodic syndromes (CAPS), Familial Cold Autoinflammatory Syndrome (FCAS), Muckle-Wells syndrome (MWS), Neonatal-Onset Multisystem Inflammatory Disease (NOMID), Deficiency of the interleukin-1 receptor antagonist (DIRA), or gouty arthritis. In some embodiments, the agent is an anti-IL1B antibody.

In one aspect, the disclosure is related to a method of determining effectiveness of an anti-IL1B antibody for treating a cancer, comprising: a) administering the anti-IL1B antibody to the animal as described herein, in some embodiments, the animal has the cancer; and b) determining inhibitory effects of the anti-IL1B antibody for treating the cancer.

In some embodiments, the cancer is a tumor, and determining the inhibitory effects of the treatment involves measuring the tumor volume in the animal.

In some embodiments, the cancer comprises one or more cancer cells that are injected into the animal.

In some embodiments, the cancer is breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, melanoma, or bone cancer. In some embodiments, the cancer is colorectal cancer, lung cancer, or melanoma.

In one aspect, the disclosure is related to a method of determining toxicity of an anti-IL1B antibody, the method comprising a) administering the anti-IL1B antibody to the animal as described herein; and b) determining weight change of the animal. In some embodiments, the method further comprises performing a blood test (e.g., determining red blood cell count).

In one aspect, the disclosure is related to a genetically-modified, non-human animal whose genome comprises at least one chromosome comprising a sequence encoding a human or chimeric interleukin 1 alpha (IL1A).

In some embodiments, the sequence encoding the human or chimeric IL1A is operably linked to an endogenous regulatory element at the endogenous IL1A gene locus in the at least one chromosome. In some embodiments, the sequence encoding a human or chimeric IL1A is operably linked to an endogenous 5′ untranslated region (5′-UTR).

In some embodiments, the sequence encoding a human or chimeric IL1A comprises a sequence encoding an amino acid sequence that is at least 70%, 75%, 80%, 85%, 90%, 95%, 99%, or 100% identical to human IL1A (SEQ ID NO: 11).

In some embodiments, the sequence comprises a sequence that is at least 70%, 75%, 80%, 85%, 90%, 95%, 99%, or 100% identical to SEQ ID NO: 32, 33, 34, or 35.

In some embodiments, the animal is a mammal, e.g., a monkey, a rodent, or a mouse. In some embodiments, the mammal is a mouse.

In some embodiments, the animal does not express endogenous IL1A.

In some embodiments, the animal has one or more cells expressing human or chimeric IL1A.

In some embodiments, the expressed human or chimeric IL1A can bind to human IL-1 receptor type I (IL1R1). In some embodiments, the expressed human or chimeric IL1A can bind to endogenous IL1R1.

In one aspect, the disclosure is related to a genetically-modified, non-human animal. In some embodiments, the genome of the animal comprises a replacement of a sequence encoding a region of endogenous IL1A with a sequence encoding a corresponding region of human IL1A at an endogenous IL1A gene locus.

In some embodiments, the sequence encoding the corresponding region of human IL1A is operably linked to an endogenous regulatory element at the endogenous IL1A locus.

In some embodiments, the animal does not express endogenous IL1A, and the animal has one or more cells expressing human or chimeric IL1A.

In some embodiments, the replaced sequence encoding a region of endogenous IL1A comprises exon 1, exon 2, exon 3, exon 4, exon 5, exon 6, and/or exon 7, or a part thereof, of endogenous IL1A gene. In some embodiments, the animal is a rodent. In some embodiments, the animal is a mouse.

In some embodiments, the animal is a mouse, and the replaced sequence starts within exon 2 and ends within exon 7 of endogenous mouse IL1A gene.

In some embodiments, the animal is heterozygous with respect to the replacement at the endogenous IL1A gene locus. In some embodiments, the animal is homozygous with respect to the replacement at the endogenous IL1A gene locus.

In one aspect, the disclosure is related to a method for making a genetically-modified, non-human animal, comprising: replacing in at least one cell of the animal, at an endogenous IL1A gene locus, a sequence encoding a region of an endogenous IL1A with a sequence encoding a corresponding region of human IL1A.

In some embodiments, the sequence encoding the corresponding region of human IL1A comprises exon 2, exon 3, exon 4, exon 5, exon 6, and/or exon 7, or a part thereof, of a human IL1A gene.

In some embodiments, the sequence encoding the corresponding region of human IL1A encodes an amino acid sequence that is at least 70%, 75%, 80%, 85%, 90%, 95%, 99%, or 100% identical to SEQ ID NO: 11.

In some embodiments, the endogenous IL1A locus comprises exon 1, exon 2, exon 3, exon 4, exon 5, exon 6, and/or exon 7, or a part thereof, of the endogenous IL1A gene. In some embodiments, the animal is a rodent. In some embodiments, the animal is a mouse. In some embodiments, the animal is a mouse, and the replaced sequence starts from within exon 2 and ends within exon 7 of endogenous mouse IL1A gene.

In one aspect, the disclosure is related to a non-human animal comprising at least one cell comprising a nucleotide sequence encoding an exogenous IL1A polypeptide. In some embodiments, the exogenous IL1A polypeptide comprises at least 50 contiguous amino acid residues that are identical to the corresponding contiguous amino acid sequence of a human IL1A. In some embodiments, the animal expresses the exogenous IL1A.

In some embodiments, the exogenous IL1A polypeptide comprises an amino acid sequence that is at least 90%, 95%, or 99% identical to SEQ ID NO: 11.

In some embodiments, the nucleotide sequence is operably linked to an endogenous IL1A regulatory element of the animal.

In some embodiments, the nucleotide sequence is integrated to an endogenous IL1A gene locus of the animal.

In some embodiments, the animal in its genome comprises, preferably from 5′ to 3′: a mouse 5′ UTR, a sequence encoding the exogenous IL1A polypeptide, and a mouse 3′ UTR.

In one aspect, the disclosure is related to a method of making a genetically-modified non-human animal cell that expresses a chimeric IL1A, the method comprising: replacing at an endogenous IL1A gene locus, a nucleotide sequence encoding a region of endogenous IL1A with a nucleotide sequence encoding a corresponding region of human IL1A, thereby generating a genetically-modified non-human animal cell that includes a nucleotide sequence that encodes the chimeric IL1A. In some embodiments, the non-human animal cell expresses the chimeric IL1A. In some embodiments, the animal is a rodent. In some embodiments, the animal is a mouse.

In some embodiments, the nucleotide sequence encoding the chimeric IL1A is operably linked to an endogenous IL1A regulatory region, e.g., promoter.

In some embodiments, the animal as described herein further comprises a sequence encoding an additional human or chimeric protein. In some embodiments, the additional human or chimeric protein is interleukin 1 beta (IL1B), IL-1 receptor type I (IL1R1), interleukin-1 receptor accessory protein (IL1RAP), programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40). In some embodiments, the additional human or chimeric protein is IL1B and the animal expresses the human or chimeric IL1B.

In some embodiments, the animal or mouse as described herein further comprises a sequence encoding an additional human or chimeric protein. In some embodiments, the additional human or chimeric protein is IL1B, IL1R1, IL1RAP, PD-1, CTLA-4, LAG-3, IL15 receptor, BTLA, PD-L1, CD3, CD27, CD28, CD47, CD137, CD154, TIGIT, TIM-3, GITR, SIRPα or OX40. In some embodiments, the additional human or chimeric protein is IL1B and the and the animal expresses the human or chimeric IL1B.

In one aspect, the disclosure is related to a method of determining effectiveness of an anti-IL1A antibody for treating an allergic disorder, comprising: a) administering the anti-IL1A antibody to the animal as described herein, in some embodiments, the animal has the allergic disorder; and b) determining effects of the anti-IL1A antibody in treating the allergic disorder.

In some embodiments, the allergic disorder is allergy, asthma, and/or atopic dermatitis.

In one aspect, the disclosure is related to a method of determining effectiveness of an anti-IL1A antibody for reducing an inflammation, comprising: a) administering the anti-IL1A antibody to the animal as described herein, in some embodiments, the animal has the inflammation; and b) determining effects of the anti-IL1A antibody for reducing the inflammation.

In one aspect, the disclosure is related to a method of determining effectiveness of an agent for treating an autoimmune disorder, comprising: a) administering the agent to the animal as described herein, in some embodiments, the animal has the autoimmune disorder; and b) determining effects of the agent for treating the autoimmune disorder. In some embodiments, the autoimmune disorder is rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

In some embodiments, the autoimmune disorder is psoriasis. In some embodiments, the agent is a corticosteroid (e.g., dexamethasone) or an anti-IL1A antibody.

In one aspect, the disclosure is related to a method of determining effectiveness of an agent for treating an autoinflammatory disease, comprising: a) administering the agent to the animal as described herein, in some embodiments, the animal has the autoinflammatory disease; and b) determining effects of the agent for treating the autoinflammatory disease.

In some embodiments, the autoinflammatory disease is tumor necrosis factor receptor associated periodic syndrome (TRAPS), hyperimmunoglobulin D syndrome (HIDS)/mevalonate kinase deficiency (MKD), familial mediterranean fever (FMF), Still's disease, adult-onset Still's disease (AOSD), autoinflammatory periodic fever syndromes, cryopyrin-associated periodic syndromes (CAPS), Familial Cold Autoinflammatory Syndrome (FCAS), Muckle-Wells syndrome (MWS), Neonatal-Onset Multisystem Inflammatory Disease (NOMID), Deficiency of the interleukin-1 receptor antagonist (DIRA), or gouty arthritis. In some embodiments, the agent is an anti-IL1A antibody or anti-IL1B antibody.

In one aspect, the disclosure is related to a method of determining effectiveness of an anti-IL1A antibody for treating a cancer, comprising: a) administering the anti-IL1A antibody to the animal as described herein, in some embodiments, the animal has the cancer; and b) determining inhibitory effects of the anti-IL1A antibody for treating the cancer.

In some embodiments, the cancer is a tumor, and determining the inhibitory effects of the treatment involves measuring the tumor volume in the animal.

In some embodiments, the cancer comprises one or more cancer cells that are injected into the animal.

In some embodiments, the cancer is a solid tumor, breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, melanoma, refractory cancer, or bone cancer.

In one aspect, the disclosure is related to a method of determining toxicity of an anti-IL1A antibody, the method comprising a) administering the anti-IL1A antibody to the animal as described herein; and b) determining weight change of the animal. In some embodiments, the method further comprises performing a blood test (e.g., determining red blood cell count).

In one aspect, the disclosure is related to a protein comprising an amino acid sequence, in some embodiments, the amino acid sequence is one of the following: (a) an amino acid sequence set forth in SEQ ID NO: 2, 4, 9, or 11; (b) an amino acid sequence that is at least 90% identical to SEQ ID NO: 2, 4, 9, or 11; (c) an amino acid sequence that is at least 91%, 92%, 93%, 94%, 95%, 96%, 97%, 98%, or 99% identical to SEQ ID NO: 2, 4, 9, or 11; (d) an amino acid sequence that is different from the amino acid sequence set forth in SEQ ID NO: 2, 4, 9, or 11 by no more than 10, 9, 8, 7, 6, 5, 4, 3, 2 or 1 amino acid; and (e) an amino acid sequence that comprises a substitution, a deletion and/or insertion of one, two, three, four, five or more amino acids to the amino acid sequence set forth in SEQ ID NO: 2, 4, 9, or 11.

In one aspect, the disclosure is related to a nucleic acid comprising a nucleotide sequence, in some embodiments, the nucleotide sequence is one of the following: (a) a sequence that encodes the protein as described herein; (b) SEQ ID NO: 1, 3, 5, 6, 7, 8, 10, 12, 13, or 14; (c) a sequence that is at least 90% identical to SEQ ID NO: 1, 3, 5, 6, 7, 8, 10, 12, 13, or 14; and (d) a sequence that is at least 91%, 92%, 93%, 94%, 95%, 96%, 97%, 98%, or 99% identical to 1, 3, 5, 6, 7, 8, 10, 12, 13, or 14.

In one aspect, the disclosure is related to a cell comprising the protein and/or the nucleic acid as described herein. In one aspect, the disclosure is related to an animal comprising the protein and/or the nucleic acid as described herein.

In one aspect, the disclosure is related to a cell comprising the protein as described herein and/or the nucleic acid as described herein. In one aspect, the disclosure is related to an animal comprising the protein as described herein and/or the nucleic acid as described herein.

The disclosure further relates to a IL1B and/or IL1A genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the IL1B and/or IL1A gene function, human IL1B antibodies, human IL1A antibodies, the drugs or efficacies for human IL1B and/or IL1A targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse IL1B gene locus.

FIG. 1B is a schematic diagram showing human IL1B gene locus.

FIG. 2 is a schematic diagram showing humanized IL1B gene locus.

FIG. 3 is a schematic diagram showing a IL1B gene targeting strategy.

FIG. 4 shows PCR identification results of cells after recombination. D01, D02, D03, D04, D05, and D06 are clone numbers. M is a marker. PC is a positive control. WT is a wild-type control. H₂O is a water control.

FIG. 5 shows Southern Blot results of cells after recombination. D01, D02, D03, D04, D05, and D06 are clone numbers. WT is a wild-type control.

FIG. 6 is a schematic diagram showing the FRT recombination process.

FIG. 7 shows PCR identification results of F1 generation mice by primers IL1B-F and IL1B-R. BF1-1, BF1-2, BF1-3, BF1-4, BF1-5, BF1-6, BF1-7, BF1-8, BF1-9, BF1-10, BF1-11, BF1-12, and BF1-13 are mouse numbers. M is a marker. PC1 and PC2 are positive controls. WT is a wild-type control. H₂O is a water control.

FIG. 8 shows PCR identification results of F1 generation mice by primers IL1B-F1 and IL1B-R. BF1-1, BF1-2, BF1-3, BF1-4, BF1-5, BF1-6, BF1-7, BF1-8, BF1-9, BF1-10, BF1-11, BF1-12, and BF1-13 are mouse numbers. M is a marker. PC1 and PC2 are positive controls. WT is a wild-type control. H₂O is a water control.

FIG. 9 shows PCR identification results of F1 generation mice (Neo cassette-removed) by primers Frt-F and Frt-R. BF1-1, BF1-2, BF1-3, BF1-4, BF1-5, BF1-6, BF1-7, BF1-8, BF1-9, BF1-10, BF1-11, BF1-12, and BF1-13 are mouse numbers. M is a marker. PC1 and PC2 are positive controls. WT is a wild-type control. H₂O is a water control.

FIG. 10A shows ELISA detection results of mouse IL1B in wild-type C57BL/6 mice and IL1B humanized heterozygous mice.

FIG. 10B shows ELISA detection results of human IL1B in wild-type C57BL/6 mice and IL1B humanized heterozygous mice.

FIG. 11A is a schematic diagram showing mouse IL1A gene locus.

FIG. 11B is a schematic diagram showing human IL1A gene locus.

FIG. 12 is a schematic diagram showing humanized IL1A gene locus.

FIG. 13 is a schematic diagram showing a IL1A gene targeting strategy.

FIG. 14 shows Southern Blot results of cells after recombination. E01, E02, E03, E04, E05, E06, and E07 are clone numbers. WT is a wild-type control.

FIG. 15 is a schematic diagram showing the FRT recombination process.

FIG. 16A shows PCR identification results of F1 generation mice by primers IL1A WT-F and IL1A WT-R. PC is a positive control. WT is a wild-type control. M is a marker. H₂O is a water control.

FIG. 16B shows PCR identification results of F1 generation mice by primers IL1A Mut-F and IL1A WT-R. PC is a positive control. WT is a wild-type control. M is a marker. H₂O is a water control.

FIG. 16C shows PCR identification results of F1 generation mice by primers IL1A Frt-F and IL1A Frt-R. PC is a positive control. WT is a wild-type control. M is a marker. H₂O is a water control.

FIG. 16D shows PCR identification results of F1 generation mice by primers IL1A Flp-F2 and IL1A Flp-R2. PC is a positive control. WT is a wild-type control. M is a marker. H₂O is a water control.

FIG. 17A shows ELISA detection results of mouse IL1B in wild-type C57BL/6 mice and IL1B humanized homozygous mice.

FIG. 17B shows ELISA detection results of human IL1B in wild-type C57BL/6 mice and IL1B humanized homozygous mice.

FIG. 18A shows ELISA detection results of mouse IL1A in wild-type C57BL/6 mice and IL1B humanized heterozygous mice.

FIG. 18B shows ELISA detection results of human IL1A in wild-type C57BL/6 mice and IL1B humanized heterozygous mice.

FIG. 19 shows the average body weight of humanized IL1B homozygous mice that were xenografted with mouse colon cancer cells (MC38), and then treated with anti-human IL1B antibody Canakinumab at 20 mg/kg.

FIG. 20 shows the percentage change of average body weight of humanized IL1B homozygous mice that were xenografted with mouse colon cancer cells (MC38), and then treated with anti-human IL1B antibody Canakinumab at 20 mg/kg.

FIG. 21 shows the average tumor volume of humanized IL1B homozygous mice that were xenografted with mouse colon cancer cells (MC38), and then treated with anti-human IL1B antibody Canakinumab at 20 mg/kg.

FIG. 22 shows the alignment between mouse IL1B amino acid sequence (NP_032387.1; SEQ ID NO: 2) and human IL1B amino acid sequence (NP_000567.1; SEQ ID NO: 4).

FIG. 23 shows the alignment between rat IL1B amino acid sequence (NP_113700.2; SEQ ID NO: 49) and human IL1B amino acid sequence (NP_000567.1; SEQ ID NO: 4).

FIG. 24 shows the alignment between mouse IL1A amino acid sequence (NP_034684.2; SEQ ID NO: 9) and human IL1A amino acid sequence (NP_000566.3; SEQ ID NO: 11).

FIG. 25 shows the alignment between rat IL1A amino acid sequence (NP_058715.1; SEQ ID NO: 50) and human IL1A amino acid sequence (NP_000566.3; SEQ ID NO: 11).

### DETAILED DESCRIPTION

This disclosure relates to transgenic non-human animal with human or chimeric (e.g., humanized) IL1B and/or IL1A, and methods of use thereof.

The interleukin-1 (IL-1) family of cytokines and receptors is unique in immunology because the IL-1 family and Toll-like receptor (TLR) families share similar functions. More than 95% of living organisms use innate immune mechanisms for survival whereas less than 5% depend on T- and B-cell functions. Innate immunity is manifested by inflammation, which can function as a mechanism of host defense but when uncontrolled is detrimental to survival. More than any other cytokine family, the IL-1 family is primarily associated with innate immunity. There are 11 members of the IL-1 family of cytokines and 10 members of the IL-1 family of receptors. More than any other cytokine family, the interleukin-1 family members are closely linked to damaging inflammation; however, the same members also function to increase nonspecific resistance to infection and development of the immune response to foreign antigens.

The IL-1 family of cytokines and receptors broadly affects a broad spectrum of immunological and inflammatory responses. The 11 members of the IL-1 family are divided into 3 subfamilies based on the IL-1 consensus sequence and the primary ligand binding receptor. With the exception of IL-1Ra, all members of the IL-1 family lack a signal peptide and are not readily secreted. They are found diffusely in the cytoplasm as precursors, and each precursor contains a three-amino acid conserved consensus sequence A-X-D, in which A may be any aliphatic amino acid, followed by any amino acid (X) and then D for aspartic acid. Nine amino acids before the consensus sequence is the N-terminal amino acid, which provides the optimal folding of the cytokine into the barrel shape for receptor binding. In the case of the IL1B precursor, nine amino acids before the consensus motif (Leu-Arg-Asp) is the caspase-1 cleavage site creating the N-terminus for optimal IL-1β bioactivity.

IL1A and IL1B bind the same receptor, the type 1 IL-1 receptor (IL-1R), recruiting both the IL-1R accessory protein and the adaptor protein MyD88 to the receptor complex, resulting in activation of the downstream signaling cascade and ultimately in the activation of a myriad of immune and inflammatory genes. Both IL1A and IL1B exist as pro-forms and cleaved forms, but whereas both forms of IL1A are biologically active, only the cleaved form of IL1B acts as a pyrogen. IL1A is grouped in a category of dual function cytokines (with IL-33 and IL-37), as it is located both within the nucleus of the cell where it plays a role in transcription, and also as a functional membrane bound cytokine. In addition, IL1A is released from necrotic cells allowing it to function as an “alarmin.” In contrast, the processing and bioavailability of IL1B is very tightly controlled. IL1B requires a “two-signal” process to become activated, with the initial priming signal triggering transcription of the gene and the second signal, resulting in inflammasome activation, allowing caspase-1 mediated cleavage and activation of IL1B.

A detailed description of the IL-1 family and its function can be found, e.g., in Dinarello, Charles A. “Overview of the IL-1 family in innate inflammation and acquired immunity.” Immunological Reviews 281.1 (2018): 8-27; and Baker, Kevin J. et al., “IL-1 family members in cancer; two sides to every story.” Frontiers in Immunology 10 (2019): 1197; each of which is incorporated herein by reference in its entirety. Thus, antibodies targeting the IL-1 family members can be potentially used to treat immune disorders (e.g., psoriasis) or cancers.

Experimental animal models are an indispensable research tool for studying the effects of these antibodies (e.g., IL1B or IL1A antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

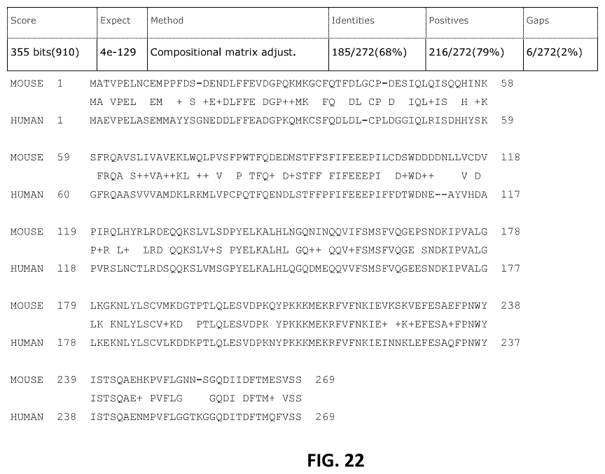

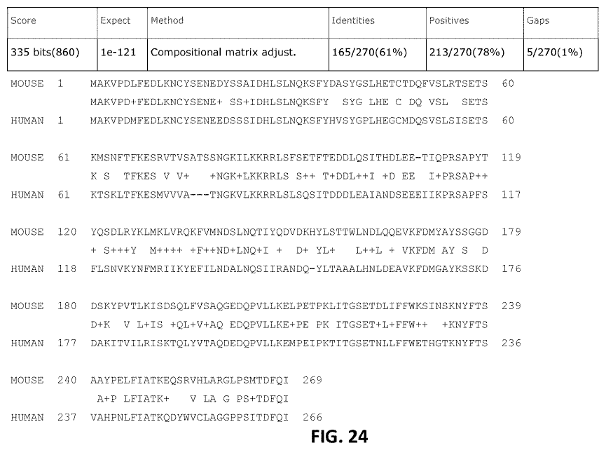

Particularly, the present disclosure demonstrates that a replacement with human IL1B sequence at an endogenous IL1B locus under control of endogenous regulatory elements provides a physiologically appropriate expression pattern and level that results in a useful humanized animal. As shown in the present disclosure, while the human IL1B sequence is quite different from the animal IL1B sequence (see e.g., FIG. 22), the human IL1B gene sequences are properly spliced in the animal, and the expressed human IL1B is functional and can properly interact with the endogenous IL1B receptor. The present disclosure also demonstrates that a replacement with human IL1A sequence at an endogenous IL1A locus under control of endogenous regulatory elements provides a physiologically appropriate expression pattern and level that results in a useful humanized animal. As shown in the present disclosure, while the human IL1A sequence is quite different from the animal IL1A sequence (see e.g., FIG. 24), the human IL1A gene sequences are properly spliced in the animal, and the expressed human IL1A is functional and can properly interact with the endogenous IL1A receptor. Both genetically modified animals that are heterozygous or homozygous for humanized IL1B and/or IL1A are grossly normal and can be used to evaluate the efficacy of anti-human IL1B or anti-human IL1A antibodies in an immune disorder model.

Unless otherwise specified, the practice of the methods described herein can take advantage of the techniques of cell biology, cell culture, molecular biology, transgenic biology, microbiology, recombinant DNA and immunology.

### IL1B

Interleukin 1 beta (IL-1B, IL-1(3, or IL1B), also known as leukocytic pyrogen, leukocytic endogenous mediator, mononuclear cell factor, lymphocyte activating factor is a cytokine protein that in humans is encoded by the IL1B gene. IL1B precursor is cleaved by cytosolic caspase 1 (interleukin 1 beta convertase) to form mature IL1B. IL1B is the most studied member of the IL-1 family due to its role in mediating autoinflammatory diseases. IL1B evolved to assist host defense against infection and a low dose of recombinant IL1B was shown to protect mice against lethal bacterial infection in the absence of neutrophils.

Unlike IL1A, IL1B is expressed in a more limited number of cell types and must be processed from its precursor form to become an active agonist in IL-1 signaling. IL1B is transcribed by monocytes, macrophages, and dendritic cells following Toll-like receptor (TLR) activation by pathogen-associated molecular patterns (PAMPs) or cytokine signaling. IL1B is also transcribed in the presence of itself in a form of auto-inflammatory induction. The inactive IL1B precursor needs to be processed by caspase-1 cleavage, which in turn requires activation by danger-associated molecular patterns (DAMPs).

IL1B is mainly produced by myeloid cells. It is synthesized as an inactive form, pro-IL1B that is activated intracellularly by caspase 1. Under normal conditions, IL-1β is secreted in low levels, and its expression and/or caspase 1-mediated activation increases under disease. In autoinflammatory diseases, high IL1B tissue levels are usually accompanied by an increase in blood levels given that monocytes release more processed IL1B. Secreted IL1B binds to its IL-1 receptor 1 (IL-1R1) and triggers a signaling cascade that controls gene expression of multiple transcription factors, growth factors and other interleukins involved in hematological function. Thereby, IL1B plays an important role in innate and adaptive immune cellular responses. It stimulates maturation of T cells and enhances proliferation of B cells. Further, IL1B promotes expression of inflammatory molecules such as cyclooxygenase type 2, type 2 phospholipase A, prostaglandin E2, platelet activating factor and nitric oxide, among others.

A detailed description of IL1B and its function can be found, e.g., in Arranz, Lorena, Maria del Mar Arriero, and Alicia Villatoro. “Interleukin-1β as emerging therapeutic target in hematological malignancies and potentially in their complications.” Blood reviews 31.5 (2017): 306-317; Rébé, Cedric, and François Ghiringhelli. “Interleukin-1β and Cancer.” Cancers 12.7 (2020): 1791; and Fields, James K. et al., “Structural basis of IL-1 family cytokine signaling.” Frontiers in Immunology 10 (2019): 1412; each of which is incorporated by reference in its entirety.

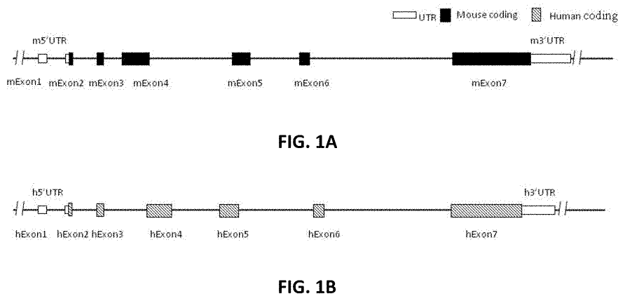

In human genomes, IL1B gene (Gene ID: 3553) locus has 7 exons, exon 1, exon 2, exon 3, exon 4, exon 5, exon 6, and exon 7 (FIG. 1B). The nucleotide sequence for human IL1B mRNA is NM 000576.3 (SEQ ID NO: 3), and the amino acid sequence for human IL1B is NP_000567.1 (SEQ ID NO: 4). The location for each exon and each region in human IL1B nucleotide sequence and amino acid sequence is listed below:

TABLE 1

| Human IL1B | NM_000576.3 | NP_000567.1 |
|---|---|---|
| (approximate | 1507bp | 269aa |
| location) | SEQ ID NO: 3 | SEQ ID NO: 4 |
| Exon 1 | 1-72 | — |
| Exon 2 | 73-134 | 1-16 |
| Exon 3 | 135-186 | 17-33 |
| Exon 4 | 187-388 | 34-100 |
| Exon 5 | 389-553 | 101-155 |
| Exon 6 | 554-684 | 156-199 |
| Exon 7 | 685-1507 | 200-269 |
| | | 1-116 |
| | | (N-terminal propeptide) |
| | | 117-269 |
| | | (C-terminal mature IL1B) |
| Replaced region | 88-897 | 1-269 |
| in Example | | |

The human IL1B gene (Gene ID: 3553) is located in Chromosome 2 of the human genome, which is located from 112829751 to 112836843 of NC 000002.12 (GRCh38.p13 (GCF_000001405.39)). The 5′-UTR is from 112,836,779 to 112,836,230, exon 1 is from 112,836,779 to 112,836,708, the first intron is from 112,836,707 to 112,836,245, exon 2 is from 112,836,244 to 112,836,183, the second intron is from 112,836,182 to 112,835,618, exon 3 is from 112,835,617 to 112,835,566, the third intron is from 112,835,565 to 112,833,576, exon 4 is from 112,833,575 to 112,833,374, the forth intron is from 112,833,373 to 112,832,827, exon 5 is from 112,832,826 to 112,832,662, the fifth intron is from 112,832,661 to 112,831,423, exon 6 is from 112,831,422 to 112,831,292, the sixth intron is from 112,831,291 to 112,830,574, exon 7 is from 112,830,573 to 112,829,751, and the 3′-UTR is from 112830360 to 112,829,751, based on transcript NM 000576.3. All relevant information for human IL1B locus can be found in the NCBI website with Gene ID: 3553, which is incorporated by reference herein in its entirety.

Human IL1B is synthesized as an inactive precursor that is cleaved by IL-1 converting enzyme (ICE) between Asp116 and Ala117 to form C-terminal mature IL1B and N-terminal IL1B propeptide. Therefore, an N-terminal propeptide (or propeptide) corresponds to amino acids 1-116 of SEQ ID NO: 4, and a C-terminal mature IL1B corresponds to amino acids 117-269 of SEQ ID NO: 4. Details can be found, e.g., in UniProt Database (UniProt ID: P01584); Higgins, Gloria C. et al., “Interleukin 1 beta propeptide is detected intracellularly and extracellularly when human monocytes are stimulated with LPS in vitro.” The Journal of Experimental Medicine 180.2 (1994): 607-614; and Afonina, Inna S., et al., “Proteolytic processing of interleukin-1 family cytokines: variations on a common theme.” Immunity 42.6 (2015): 991-1004; each of which is incorporated herein by reference in its entirety.

In mice, IL1B gene locus has 7 exons, exon 1, exon 2, exon 3, exon 4, exon 5, exon 6, and exon 7 (FIG. 1A). The nucleotide sequence for mouse IL1B mRNA is NM 008361.4 (SEQ ID NO: 1), the amino acid sequence for mouse IL1B is NP_032387.1 (SEQ ID NO: 2). The location for each exon and each region in the mouse IL1B nucleotide sequence and amino acid sequence is listed below:

TABLE 2

| Mouse IL1B | NM_008361.4 | NP_032387.1 |
|---|---|---|
| (approximate | 1348bp | 269aa |
| location) | SEQ ID NO: 1 | SEQ ID NO: 2 |
| Exon 1 | 1-72 | — |
| Exon 2 | 73-134 | 1-16 |
| Exon 3 | 135-183 | 17-32 |
| Exon 4 | 184-385 | 33-99 |
| Exon 5 | 386-556 | 100-156 |
| Exon 6 | 557-687 | 157-200 |
| Exon 7 | 688-1348 | 201-269 |
| | | 1-117 |
| | | (N-terminal propetide) |
| | | 118-269 |
| | | (C-terminal mature IL1B) |
| Replaced region | 88-897 | 1-269 |
| in Example | | |

The mouse IL1B gene (Gene ID: 16176) is located in Chromosome 2 of the mouse genome, which is located from 129364569 to 129371164 of NC 000068.7 (GRCm38.p6 (GCF_000001635.26)). The 5′-UTR is from 129,371,139 to 129,370,331, exon 1 is from 129,371,139 to 129,371,068, the first intron is from 129,371,067 to 129,370,346, exon 2 is from 129,370,345 to 129,370,284, the second intron is from 129,370,283 to 129,369,752, exon 3 is from 129,369,751 to 129,369,703, the third intron is from 129,369,702 to 129,368,159, exon 4 is from 129,368,158 to 129,367,957, the forth intron is from 129,367,956 to 129,367,411, exon 5 is from 129,367,410 to 129,367,240, the fifth intron is from 129,367,239 to 129,366,091, exon 6 is from 129,366,090 to 129,365,960, the sixth intron is from 129,365,959 to 129,365,239, exon 7 is from 129,365,238 to 129,364,570, and the 3′-UTR is from 129365028 to 129,364,570, based on transcript NM 008361.4. All relevant information for mouse IL1B locus can be found in the NCBI website with Gene ID: 16176, which is incorporated by reference herein in its entirety.

According to the UniProt Database (UniProt ID: P10749), an N-terminal propeptide (or propeptide) corresponds to amino acids 1-117 of SEQ ID NO: 2, and a C-terminal mature IL1B corresponds to amino acids 118-269 of SEQ ID NO: 2.

FIG. 22 shows the alignment between mouse IL1B amino acid sequence (NP_032387.1; SEQ ID NO: 2) and human IL1B amino acid sequence (NP_000567.1; SEQ ID NO: 4). Thus, the corresponding amino acid residue or region between human and mouse IL1B can be found in FIG. 22.

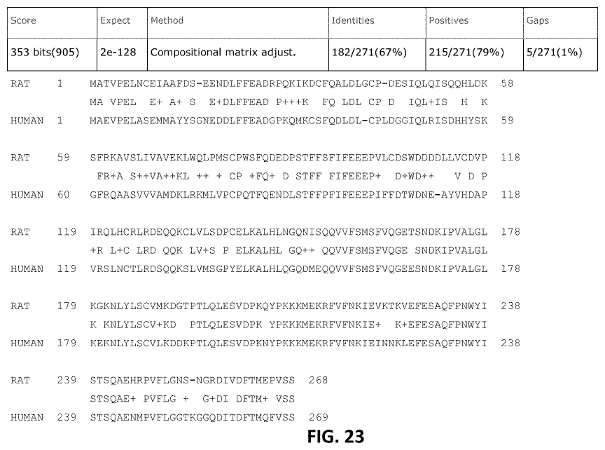

IL1B genes, proteins, and locus of the other species are also known in the art. For example, the gene ID for IL1B in Rattus norvegicus (rat) is 24494, the gene ID for IL1B in Macaca mulatta (Rhesus monkey) is 704701, the gene ID for IL1B in Sus scrofa (pig) is 397122, the gene ID for IL1B in Oryctolagus cuniculus (rabbit) is 100008990, and the gene ID for IL1B in Felis catus (domestic cat) is 768274. The relevant information for these genes (e.g., intron sequences, exon sequences, amino acid residues of these proteins) can be found, e.g., in NCBI database, which is incorporated by reference herein in its entirety. FIG. 23 shows the alignment between rodent IL1B amino acid sequence (NP_113700.2; SEQ ID NO: 49) and human IL1B amino acid sequence (NP_000567.1; SEQ ID NO: 4). Thus, the corresponding amino acid residue or region between rodent and human IL1B can be found in FIG. 23.

The present disclosure provides human or chimeric (e.g., humanized) IL1B nucleotide sequence and/or amino acid sequences. In some embodiments, the entire sequence of mouse exon 1, exon 2, exon 3, exon 4, exon 5, exon 6, exon 7, propeptide, and/or mature IL1B, are replaced by the corresponding human sequence. In some embodiments, a “region” or a “portion” of mouse exon 1, exon 2, exon 3, exon 4, exon 5, exon 6, exon 7, propeptide, and/or mature IL1B, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, or 260 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, propeptide, or mature IL1B. In some embodiments, a region, a portion, or the entire sequence of mouse exon 1, exon 2, exon 3, exon 4, exon 5, exon 6, and/or exon 7 (e.g., a portion of exon 2, exon 3, exon 4, exon 5, exon 6, and a portion of exon 7 of mouse IL1B gene) are replaced by human exon 1, exon 2, exon 3, exon 4, exon 5, exon 6, and/or exon 7 (e.g., a portion of exon 2, exon 3, exon 4, exon 5, exon 6, and a portion of exon 7 of human IL1B gene) sequence.

In some embodiments, the present disclosure also provides a chimeric (e.g., humanized) or human IL1B nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL1B mRNA sequence (e.g., SEQ ID NO: 1), mouse IL1B amino acid sequence (e.g., SEQ ID NO: 2), or a portion thereof (e.g., a portion of exon 1 and a portion of exon 7 of NM 008361.4 (SEQ ID NO: 1)); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL1B mRNA sequence (e.g., SEQ ID NO: 3), human IL1B amino acid sequence (e.g., SEQ ID NO: 4), or a portion thereof (e.g., a portion of exon 2, exon 3, exon 4, exon 5, exon 6, and a portion of exon 7, of NM 000576.3 (SEQ ID NO: 3)).

In some embodiments, the sequence encoding amino acids 1-269 of mouse IL1B (SEQ ID NO: 2) is replaced. In some embodiments, the sequence is replaced by a sequence encoding a corresponding region of human IL1B (e.g., amino acids 1-269 of human IL1B (SEQ ID NO: 4)).

In some embodiments, the sequence encoding amino acids 118-269 of mouse IL1B (SEQ ID NO: 2) is replaced. In some embodiments, the sequence is replaced by a sequence encoding a corresponding region of human IL1B (e.g., amino acids 117-269 of human IL1B (SEQ ID NO: 4)).

In some embodiments, the nucleic acid sequence described herein are operably linked to a promotor or regulatory element, e.g., an endogenous mouse IL1B promotor, an inducible promoter, an enhancer, and/or mouse or human regulatory elements. In some embodiments, the nucleic acid sequence described herein is connected to an endogenous 5′ UTR. In some embodiments, the 5′UTR is identical to nucleic acid positions 1-72 of exon 1 and positions 73-87 of exon 2 of SEQ ID NO: 1. In some embodiments, the nucleic acid sequence described herein is connected to a human 5′ UTR. In some embodiments, the nucleic acid sequence described herein is connected to an endogenous 3′ UTR. In some embodiments, the nucleic acid sequence described herein is connected to a human 3′ UTR.

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire mouse IL1B nucleotide sequence (e.g., a portion of exon 2, exons 3-6, and a portion of exon 7, of NM 008361.4 (SEQ ID NO: 1)).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse IL1B nucleotide sequence (e.g., exon 1, a portion of exon 2, and a portion of exon 7, of NM 008361.4 (SEQ ID NO: 1)).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human IL1B nucleotide sequence (e.g., exon 1, a portion of exon 2, and a portion of exon 7, of NM 000576.3 (SEQ ID NO: 3)).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL1B nucleotide sequence (e.g., a portion of exon 2, exons 3-6, and a portion of exon 7, of NM 000576.3 (SEQ ID NO: 3)).

In some embodiments, the amino acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL1B amino acid sequence (e.g., NP_032387.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL1B amino acid sequence (e.g., NP_032387.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL1B amino acid sequence (e.g., NP_000567.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL1B amino acid sequence (e.g., NP_000567.1 (SEQ ID NO: 4)).

### IL1A

Interleukin 1 alpha (IL-1A, ILla, or IL1A) also known as hematopoietin 1, is a cytokine of the interleukin 1 family that in humans is encoded by the IL1A gene. In general, Interleukin 1 is responsible for the production of inflammation, as well as the promotion of fever and sepsis.

IL1A is produced mainly by activated macrophages, as well as neutrophils, epithelial cells, and endothelial cells. It possesses metabolic, physiological, haematopoietic activities, and plays one of the central roles in the regulation of the immune responses. It binds to the interleukin-1 receptor. It is on the pathway that activates tumor necrosis factor-alpha.

The IL1A precursor gene is expressed constitutively in cells, including kidney, liver, lung, endothelial cells, astrocytes, and the epithelium of the gastrointestinal track. Unlike IL1B, IL1A is already active in its primary precursor form and acts as an alarmin by eliciting a signaling cascade through IL-1RI. Similar to other cytokines within the IL-1 family, IL1A is composed of 12 β-strands in a β-trefoil architecture.

IL1A is a “dual-function” cytokine. Dual-function cytokines are found in the nucleus where they bind to DNA and serve a function; the same cytokine binds to its cell membrane receptor and initiates signal transduction. There is a nuclear localization sequence in the precursor region of the cytokine and IL1A in the nucleus acts as a transcription factor. In that context, nuclear IL1A functions to increase gene expression, for example the chemokine IL-8. Nuclear translocation of IL1A can also be a sink for its pro-inflammatory properties. For example, the IL1A precursor shuttles between the cytosol and the nucleus within a few nanoseconds. When the cell is exposed to a proapoptotic signal, IL1A leaves the cytosolic pool and rapidly migrates to the nucleus where it binds tightly to chromatin and fails to induce inflammation. In contrast, when the cell is exposed to a necrotic signal, IL1A migrates from nucleus to the cytosol and the lysates of these cells are highly inflammatory. In general, when the precursor of IL1A is released from necrotic cells, IL1A is a DAMP and evokes a broad number of inflammatory reactions via the IL-1R1.

A detailed description of IL1A and its function can be found, e.g., in Di Paolo et al., “Interleukin 1α and the inflammatory process.” Nature Immunology 17.8 (2016): 906-913; Fields, James K., Sebastian Günther, and Eric J. Sundberg. “Structural basis of IL-1 family cytokine signaling.” Frontiers in Immunology 10 (2019): 1412; and Dinarello, Charles A. “Overview of the IL-1 family in innate inflammation and acquired immunity.” Immunological Reviews 281.1 (2018): 8-27; each of which is incorporated by reference in its entirety.

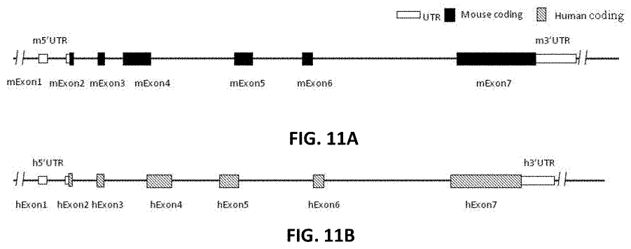

In human genomes, IL1A gene (Gene ID: 3552) locus has 7 exons, exon 1, exon 2, exon 3, exon 4, exon 5, exon 6, and exon 7 (FIG. 11B). The nucleotide sequence for human IL1A mRNA is NM 000575.5 (SEQ ID NO: 10), and the amino acid sequence for human IL1A is NP_000566.3 (SEQ ID NO: 11). The location for each exon and each region in human IL1A nucleotide sequence and amino acid sequence is listed below:

TABLE 3

| Human IL1A | NM_000575.5 | NP_000566.3 |
|---|---|---|
| (approximate | 2017bp | 271aa |
| location) | SEQ ID NO: 10 | SEQ ID NO: 11 |
| Exon 1 | 1-51 | — |
| Exon 2 | 52-106 | 1-15 |
| Exon 3 | 107-155 | 16-32 |
| Exon 4 | 156-378 | 33-106 |
| Exon 5 | 379-549 | 107-163 |
| Exon 6 | 550-674 | 164-205 |
| Exon 7 | 675-2017 | 206-271 |
| | | 1-112 |
| | | (N-terminal propeptide) |
| | | 113-271 |
| | | (C-terminal mature IL1A) |
| Replaced region | 59-875 | 1-271 |
| in Example | | |

The human IL1A gene (Gene ID: 3552) is located in Chromosome 2 of the human genome, which is located from 112773925 to 112784493 of NC 000002.12 (GRCh38.p13 (GCF_000001405.39)). The 5′-UTR is from 112,784,493 to 112,783,771, exon 1 is from 112,784,493 to 112,784,443, the first intron is from 112,784,442 to 112,783,779, exon 2 is from 112,783,778 to 112,783,724, the second intron is from 112,783,723 to 112,782,765, exon 3 is from 112,782,764 to 112,782,716, the third intron is from 112,782,715 to 112,781,827, exon 4 is from 112,781,826 to 112,781,604, the forth intron is from 112,781,603 to 112,779,667, exon 5 is from 112,779,666 to 112,779,496, the fifth intron is from 112,779,495 to 112,778,112, exon 6 is from 112,778,111 to 112,777,987, the sixth intron is from 112,777,986 to 112,775,268, exon 7 is from 112,775,267 to 112,773,925, the 3′-UTR is from 112775066 to 112,773,925, based on transcript NM 000575.5. All relevant information for human IL1A locus can be found in the NCBI website with Gene ID: 3552, which is incorporated by reference herein in its entirety.

Similar to IL1B, human IL1A can be cleaved to form C-terminal mature IL1A and N-terminal IL1A propeptide. The N-terminal propeptide (or propeptide) corresponds to amino acids 1-112 of SEQ ID NO: 11, and a C-terminal mature IL1A corresponds to amino acids 113-271 of SEQ ID NO: 11. Details can be found, e.g., in UniProt Database (UniProt ID: P01583); and Afonina, Inna S., et al., “Proteolytic processing of interleukin-1 family cytokines: variations on a common theme.” Immunity 42.6 (2015): 991-1004; each of which is incorporated herein by reference in its entirety.

In mice, IL1A gene locus has 7 exons, exon 1, exon 2, exon 3, exon 4, exon 5, exon 6, and exon 7 (FIG. 11A). The nucleotide sequence for mouse IL1A mRNA is NM 010554.4 (SEQ ID NO: 8), the amino acid sequence for mouse IL1A is NP_034684.2 (SEQ ID NO: 9). The location for each exon and each region in the mouse IL1A nucleotide sequence and amino acid sequence is listed below:

TABLE 4

| Mouse IL1A | NM_010554.4 | NP_034684.2 |
|---|---|---|
| (approximate | 1974bp | 270aa |
| location) | SEQ ID NO: 8 | SEQ ID NO: 9 |
| Exon 1 | 1-52 | — |
| Exon 2 | 53-107 | 1-16 |
| Exon 3 | 108-156 | 17-32 |
| Exon 4 | 157-385 | 33-108 |
| Exon 5 | 386-559 | 109-166 |
| Exon 6 | 560-684 | 167-208 |
| Exon 7 | 685-1974 | 209-270 |
| | | 1-114 |
| | | (N-terminal propeptide) |
| | | 115-270 |
| | | (C-terminal mature IL1A) |
| Replaced region | 61-873 | 1-270 |
| in Example | | |

The mouse IL1A gene (Gene ID: 16175) is located in Chromosome 2 of the mouse genome, which is located from 129299609 to 129310186 of NC 000068.7 (GRCm38.p6 (GCF_000001635.26)). The 5′-UTR is from 129,309,972 to 129,309,102, exon 1 is from 129,309,972 to 129,309,921, the first intron is from 129,309,920 to 129,309,110, exon 2 is from 129,309,109 to 129,309,055, the second intron is from 129,309,054 to 129,307,932, exon 3 is from 129,307,931 to 129,307,883, the third intron is from 129,307,882 to 129,306,693, exon 4 is from 129,306,692 to 129,306,464, the forth intron is from 129,306,463 to 129,304,847, exon 5 is from 129,304,846 to 129,304,673, the fifth intron is from 129,304,672 to 129,302,998, exon 6 is from 129,302,997 to 129,302,873, the sixth intron is from 129,302,872 to 129,300,900, exon 7 is from 129,300,899 to 129,299,610, the 3′-UTR is from 129300710 to 129,299,610, based on transcript NM 010554.4. All relevant information for mouse IL1A locus can be found in the NCBI website with Gene ID: 16175, which is incorporated by reference herein in its entirety.

According to the UniProt Database (UniProt ID: P01582), an N-terminal propeptide (or propeptide) corresponds to amino acids 1-114 of SEQ ID NO: 9, and a C-terminal mature IL1B corresponds to amino acids 115-270 of SEQ ID NO: 9.

FIG. 24 shows the alignment between mouse IL1A amino acid sequence (NP_034684.2; SEQ ID NO: 9) and human IL1A amino acid sequence (NP_000566.3; SEQ ID NO: 11). Thus, the corresponding amino acid residue or region between human and mouse IL1A can be found in FIG. 24.

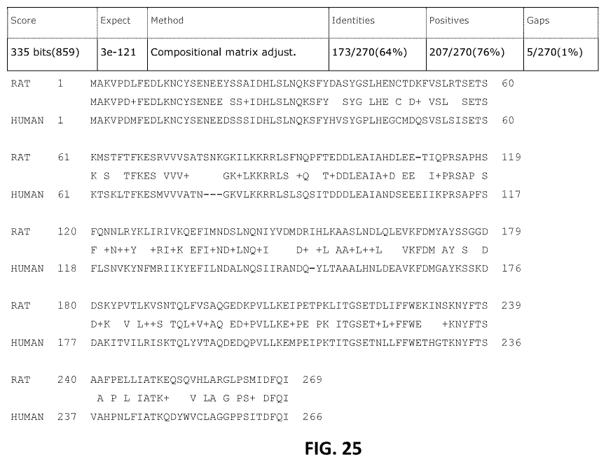

IL1A genes, proteins, and locus of the other species are also known in the art. For example, the gene ID for IL1A in Rattus norvegicus (rat) is 24493, the gene ID for IL1A in Macaca mulatta (Rhesus monkey) is 700193, the gene ID for IL1A in Sus scrofa (pig) is 397094, the gene ID for IL1A in Oryctolagus cuniculus (rabbit) is 100009250, and the gene ID for IL1A in Felis catus (domestic cat) is 493944. The relevant information for these genes (e.g., intron sequences, exon sequences, amino acid residues of these proteins) can be found, e.g., in NCBI database, which is incorporated by reference herein in its entirety. FIG. 25 shows the alignment between rodent IL1A amino acid sequence (NP_058715.1; SEQ ID NO: 50) and human IL1A amino acid sequence (NP_000566.3; SEQ ID NO: 11). Thus, the corresponding amino acid residue or region between rodent and human IL1A can be found in FIG. 25.

The present disclosure provides human or chimeric (e.g., humanized) IL1A nucleotide sequence and/or amino acid sequences. In some embodiments, the entire sequence of mouse exon 1, exon 2, exon 3, exon 4, exon 5, exon 6, exon 7, propeptide, and/or mature IL1A, are replaced by the corresponding human sequence. In some embodiments, a “region” or a “portion” of mouse exon 1, exon 2, exon 3, exon 4, exon 5, exon 6, exon 7, propeptide, and/or mature IL1A, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, or 270 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, propeptide, or mature IL1A. In some embodiments, a region, a portion, or the entire sequence of mouse exon 1, exon 2, exon 3, exon 4, exon 5, exon 6, and/or exon 7 (e.g., a portion of exon 2, exon 3, exon 4, exon 5, exon 6, and a portion of exon 7 of mouse IL1A gene) are replaced by human exon 1, exon 2, exon 3, exon 4, exon 5, exon 6, and/or exon 7 (e.g., a portion of exon 2, exon 3, exon 4, exon 5, exon 6, and a portion of exon 7 of human IL1A gene) sequence.

In some embodiments, the present disclosure also provides a chimeric (e.g., humanized) or human IL1A nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL1A mRNA sequence (e.g., SEQ ID NO: 8), mouse IL1A amino acid sequence (e.g., SEQ ID NO: 9), or a portion thereof (e.g., a portion of exon 1 and a portion of exon 7 of NM 010554.4 (SEQ ID NO: 8)); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human mRNA sequence (e.g., SEQ ID NO: 10), human IL1A amino acid sequence (e.g., SEQ ID NO: 11), or a portion thereof (e.g., a portion of exon 2, exon 3, exon 4, exon 5, exon 6, and a portion of exon 7, of NM 000575.5 (SEQ ID NO: 10)).

In some embodiments, the sequence encoding amino acids 1-270 of mouse IL1A (SEQ ID NO: 9) is replaced. In some embodiments, the sequence is replaced by a sequence encoding a corresponding region of human IL1A (e.g., amino acids 1-271 of human IL1A (SEQ ID NO: 11)).

In some embodiments, the sequence encoding amino acids 115-270 of mouse IL1A (SEQ ID NO: 9) is replaced. In some embodiments, the sequence is replaced by a sequence encoding a corresponding region of human IL1A (e.g., amino acids 113-271 of human IL1A (SEQ ID NO: 11)).

In some embodiments, the nucleic acid sequence described herein are operably linked to a promotor or regulatory element, e.g., an endogenous mouse IL1A promotor, an inducible promoter, an enhancer, and/or mouse or human regulatory elements. In some embodiments, the nucleic acid sequence described herein is connected to an endogenous 5′ UTR. In some embodiments, the 5′UTR is identical to nucleic acid positions 1-52 of exon 1 and positions 53-60 of exon 2 of SEQ ID NO: 8. In some embodiments, the nucleic acid sequence described herein is connected to a human 5′ UTR. In some embodiments, the nucleic acid sequence described herein is connected to an endogenous 3′ UTR. In some embodiments, the nucleic acid sequence described herein is connected to a human 3′ UTR.

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire mouse IL1A nucleotide sequence (e.g., a portion of exon 2, exons 3-6, and a portion of exon 7, of NM 010554.4 (SEQ ID NO: 8)).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse IL1A nucleotide sequence (e.g., exon 1, a portion of exon 2, and a portion of exon 7, of NM 010554.4 (SEQ ID NO: 8)).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human IL1A nucleotide sequence (e.g., exon 1, a portion of exon 2, and a portion of exon 7, of NM 000575.5 (SEQ ID NO: 10)).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL1A nucleotide sequence (e.g., a portion of exon 2, exons 3-6, and a portion of exon 7, of NM 000575.5 (SEQ ID NO: 10)).

In some embodiments, the amino acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL1A amino acid sequence (e.g., NP_034684.2 (SEQ ID NO: 9)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL1A amino acid sequence (e.g., NP_034684.2 (SEQ ID NO: 9)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL1A amino acid sequence (e.g., NP_000566.3 (SEQ ID NO: 11)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL1A amino acid sequence (e.g., NP_000566.3 (SEQ ID NO: 11)).

The present disclosure also provides a human or humanized IL1B amino acid sequence, or a human or humanized IL1A amino acid sequence, wherein the amino acid sequence is selected from the group consisting of:

a) an amino acid sequence shown in SEQ ID NO: 2, 4, 9, or 11;

b) an amino acid sequence having a homology of at least 90% with or at least 90% identical to the amino acid sequence shown in SEQ ID NO: 2, 4, 9, or 11;

c) an amino acid sequence encoded by a nucleic acid sequence, wherein the nucleic acid sequence is able to hybridize to a nucleotide sequence encoding the amino acid shown in SEQ ID NO: 2, 4, 9, or 11, under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2, 4, 9, or 11;

e) an amino acid sequence that is different from the amino acid sequence shown in SEQ ID NO: 2, 4, 9, or 11, by no more than 10, 9, 8, 7, 6, 5, 4, 3, 2 or no more than 1 amino acid; or f) an amino acid sequence that comprises a substitution, a deletion and/or insertion of one or more amino acids to the amino acid sequence shown in SEQ ID NO: 2, 4, 9, or 11.

The present disclosure also relates to a IL1B nucleic acid (e.g., DNA or RNA) sequence, or a IL1A nucleic acid (e.g., DNA or RNA) sequence, wherein the nucleic acid sequence can be selected from the group consisting of:

a) a nucleic acid sequence as shown in SEQ ID NO: 1, 3, 5, 6, 7, 8, 10, 12, 13, or 14; a nucleic acid sequence encoding a homologous IL1B amino acid sequence of a humanized mouse IL1B; or a nucleic acid sequence encoding a homologous IL1A amino acid sequence of a humanized mouse IL1A;

b) a nucleic acid sequence that is shown in SEQ ID NO: 1, 3, 5, 6, 7, 8, 10, 12, 13, or 14;

c) a nucleic acid sequence that is able to hybridize to the nucleotide sequence as shown in SEQ ID NO: 1, 3, 5, 6, 7, 8, 10, 12, 13, or 14 under a low stringency condition or a strict stringency condition;

d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 1, 3, 5, 6, 7, 8, 10, 12, 13, or 14;

e) a nucleic acid sequence that encodes an amino acid sequence, wherein the amino acid sequence has a homology of at least 90% with or at least 90% identical to the amino acid sequence shown in SEQ ID NO: 2, 4, 9, or 11;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2, 4, 9, or 11;

g) a nucleic acid sequence that encodes an amino acid sequence, wherein the amino acid sequence is different from the amino acid sequence shown in SEQ ID NO: 2, 4, 9, or 11 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 2, 4, 9, or 11.

The present disclosure also relates to a IL1B nucleic acid (e.g., DNA or RNA) sequence, wherein the nucleic acid sequence can be selected from the group consisting of:

a) the transcribed mRNA sequence is all or part of the nucleotide sequence shown in positions 88-897 of SEQ ID NO: 3;

b) the transcribed mRNA sequence is at least 70%, 80%, 90%, 91%, 92%, 93%, 94%, 95%, 96%, 97%, 98% or at least 99% identical to the nucleotide sequence shown in positions 88-897 of SEQ ID NO: 3;

c) the transcribed mRNA sequence differs from the nucleotide sequence shown in positions 88-897 of SEQ ID NO: 3 by no more than 10, 9, 8, 7, 6, 5, 4, 3, 2 or no more than 1 nucleotide; and

d) the transcribed mRNA sequence is shown in the nucleotide sequence shown at positions 88-897 of SEQ ID NO: 3, including the nucleotide sequence of substitution, deletion and/or insertion of one or more nucleotides.

The present disclosure also relates to a IL1A nucleic acid (e.g., DNA or RNA) sequence, wherein the nucleic acid sequence can be selected from the group consisting of:

a) the transcribed mRNA sequence is all or part of the nucleotide sequence shown in positions 59-875 of SEQ ID NO: 10;

b) the transcribed mRNA sequence is at least 70%, 80%, 90%, 91%, 92%, 93%, 94%, 95%, 96%, 97%, 98% or at least 99% identical to the nucleotide sequence shown in positions 59-875 of SEQ ID NO: 10;

c) the transcribed mRNA sequence differs from the nucleotide sequence shown in positions 59-875 of SEQ ID NO: 10 by no more than 10, 9, 8, 7, 6, 5, 4, 3, 2 or no more than 1 nucleotide; and

d) the transcribed mRNA sequence is shown in the nucleotide sequence shown at positions 59-875 of SEQ ID NO: 10, including the nucleotide sequence of substitution, deletion and/or insertion of one or more nucleotides.

The present disclosure further relates to an IL1B genomic DNA sequence of a humanized mouse IL1B, or an IL1A genomic DNA sequence of a humanized mouse IL1A. The DNA sequence is obtained by a reverse transcription of the mRNA obtained by transcription thereof is consistent with or complementary to the DNA sequence homologous to the sequence shown in SEQ ID NO: 5 or 12.

The disclosure also provides an amino acid sequence that has a homology of at least 90% with, or at least 90% identical to the sequence shown in SEQ ID NO: 2, 4, 9, or 11, and has protein activity. In some embodiments, the homology with the sequence shown in SEQ ID NO: 2, 4, 9, or 11, is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 2, 4, 9, or 11, is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 1, 3, or 5, and encodes a polypeptide that has IL1B protein activity. In some embodiments, the homology with the sequence shown in SEQ ID NO: 1, 3, or 5 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 8, 10, or 12, and encodes a polypeptide that has IL1A protein activity. In some embodiments, the homology with the sequence shown in SEQ ID NO: 8, 10, or 12 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 1, 3, 5, 6, 7, 8, 10, 12, 13, or 14 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For illustration purposes, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) IL1B and/or IL1A from an endogenous non-human IL1B locus and/or an endogenous non-human IL1A locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous IL1B and/or IL1A locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized IL1B gene or a humanized IL1B nucleic acid. In some embodiments, at least one or more portions of the gene or the nucleic acid is from the human IL1B gene. In some embodiments, the gene or the nucleic acid comprises a sequence that encodes a human or humanized IL1B protein. The encoded IL1B protein is functional or has at least one activity of the human IL1B protein and/or the non-human IL1B protein, e.g., interacting with human or non-human IL-1R1 and/or IL1RAcP; competing with IL-1Ra binding to IL1R1; inducing prostaglandin synthesis, neutrophil influx and activation; inducing T-cell activation and cytokine production; inducing B-cell activation and antibody production; inducing fibroblast proliferation and collagen production; promoting Th17 differentiation of T-cells; synergizing with IL12/interleukin-12 to induce IFNG synthesis from T-helper 1 (Th1) cells; inducing VEGF production synergistically with TNF and IL6; and/or upregulating the immune response.

In some embodiments, the chimeric gene or the chimeric nucleic acid is a humanized IL1A gene or a humanized IL1A nucleic acid. In some embodiments, at least one or more portions of the gene or the nucleic acid is from the human IL1A gene. In some embodiments, the gene or the nucleic acid comprises a sequence that encodes a human or humanized IL1A protein. The encoded IL1A protein is functional or has at least one activity of the human IL1A protein and/or the non-human IL1A protein, e.g., interacting with human or non-human or IL1RL1 and/or IL1RAcP; competing with IL1Ra binding to IL1R1; simulating fibroblasts proliferation; inducing synthesis of proteases, subsequent muscle proteolysis; releasing amino acids in blood and stimulating acute-phase proteins synthesis; changing the metallic ion content of blood plasma by increasing copper and decreasing zinc and iron concentration in blood; inducing production of SASP factors by senescent cells as a result of mTOR activity; increasing blood neutrophils; activating lymphocyte proliferation; inducing fever; and/or upregulating the immune response.

In some embodiments, the chimeric protein or the chimeric polypeptide is a humanized IL1B protein or a humanized IL1B polypeptide. In some embodiments, at least one or more portions of the amino acid sequence of the protein or the polypeptide is from a human IL1B protein. The human IL1B protein or the humanized IL1B protein is functional or has at least one activity of the human IL1B protein or the non-human IL1B protein.

In some embodiments, the chimeric protein or the chimeric polypeptide is a humanized IL1A protein or a humanized IL1A polypeptide. In some embodiments, at least one or more portions of the amino acid sequence of the protein or the polypeptide is from a human IL1A protein. The human IL1A protein or the humanized IL1A protein is functional or has at least one activity of the human IL1A protein or the non-human IL1A protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 12951/SV, 12951/SvIm), 129S2, 129S4, 129S5, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL1B and/or IL1A animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD-Prkdcscid IL-2rγ^{null} NOD mice, NOD-Rag 1−/−-IL2rg−/− (NRG) mice, Rag 2−/−-IL2rg−/− (RG) mice, SCID mice, NOD/SCID mice, IL2Rγ knockout mice, NOD/SCID/γc^{null} mice (Ito, M. et al., NOD/SCID/γc^{null} mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL1B and/or IL1A locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD-Prkdcscid IL-2rγ^{null} NOD mice, NOD-Rag 1−/-−IL2rg−/− (NRG) mice, Rag 2−/-−IL2rg−/− (RG) mice, NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety.

In some embodiments, the non-human animal (e.g., mouse) can include a replacement of all or part of mature IL1B coding sequence with human mature IL1B coding sequence. In some embodiments, the non-human animal (e.g., mouse) can include a replacement of all or part of mature IL1B coding sequence with human mature IL1B coding sequence. In some embodiments, the non-human animal (e.g., mouse) can include a replacement of all or part of mature IL1A coding sequence with human mature IL1A coding sequence. In some embodiments, the non-human animal (e.g., mouse) can include a replacement of all or part of mature IL1A coding sequence with human mature IL1A coding sequence.

In some embodiments, the genetically modified non-human animal comprises a modification of an endogenous non-human IL1B locus. In some embodiments, the modification can comprise a human nucleic acid sequence encoding at least a portion of a mature IL1B protein (e.g., at least 10%, 20%, 30%, 40%, 50%, 60%, 70%, 80%, 90%, 95%, 96%, 97%, 98%, 99% or 100% identical to the mature IL1B protein sequence). Although genetically modified cells are also provided that can comprise the modifications described herein (e.g., ES cells, somatic cells), in many embodiments, the genetically modified non-human animals comprise the modification of the endogenous IL1B locus in the germline of the animal. In some embodiments, the genetically modified non-human animal comprises a modification of an endogenous non-human IL1A locus. In some embodiments, the modification can comprise a human nucleic acid sequence encoding at least a portion of a mature IL1A protein (e.g., at least 10%, 20%, 30%, 40%, 50%, 60%, 70%, 80%, 90%, 95%, 96%, 97%, 98%, 99% or 100% identical to the mature IL1A protein sequence). Although genetically modified cells are also provided that can comprise the modifications described herein (e.g., ES cells, somatic cells), in many embodiments, the genetically modified non-human animals comprise the modification of the endogenous IL1A locus in the germline of the animal.

In some embodiments, the genetically modified mice express a human IL1B and/or a chimeric (e.g., humanized) IL1B from endogenous mouse loci, wherein the endogenous mouse IL1B gene has been replaced with a human IL1B gene and/or a nucleotide sequence that encodes a region of human IL1B sequence or an amino acid sequence that is at least 10%, 20%, 30%, 40%, 50%, 60%, 70%, 80%, 90%, 95%, 96%, 97%, 98%, or 99% identical to the human IL1B sequence. In various embodiments, an endogenous non-human IL1B locus is modified in whole or in part to comprise human nucleic acid sequence encoding at least one protein-coding sequence of a mature IL1B protein. In some embodiments, the genetically modified mice express a human IL1A and/or a chimeric (e.g., humanized) IL1A from endogenous mouse loci, wherein the endogenous mouse IL1A gene has been replaced with a human IL1A gene and/or a nucleotide sequence that encodes a region of human IL1A sequence or an amino acid sequence that is at least 10%, 20%, 30%, 40%, 50%, 60%, 70%, 80%, 90%, 95%, 96%, 97%, 98%, or 99% identical to the human IL1A sequence. In various embodiments, an endogenous non-human IL1A locus is modified in whole or in part to comprise human nucleic acid sequence encoding at least one protein-coding sequence of a mature IL1A protein.

In some embodiments, the genetically modified mice express the human IL1B and/or chimeric IL1B (e.g., humanized IL1B) from endogenous loci that are under control of mouse promoters and/or mouse regulatory elements. The replacement(s) at the endogenous mouse loci provide non-human animals that express human IL1B or chimeric IL1B (e.g., humanized IL1B) in appropriate cell types and in a manner that does not result in the potential pathologies observed in some other transgenic mice known in the art. The human IL1B or the chimeric IL1B (e.g., humanized IL1B) expressed in animal can maintain one or more functions of the wild-type mouse or human IL1B in the animal. For example, human or non-human IL1B receptors (e.g., IL1R1) can bind to the expressed IL1B, and trigger an inflammatory cascade. Furthermore, in some embodiments, the animal does not express endogenous IL1B. As used herein, the term “endogenous IL1B” refers to IL1B protein that is expressed from an endogenous IL1B nucleotide sequence of the non-human animal (e.g., mouse) before any genetic modification.

In some embodiments, the genetically modified mice express the human IL1A and/or chimeric IL1A (e.g., humanized IL1A) from endogenous loci that are under control of mouse promoters and/or mouse regulatory elements. The replacement(s) at the endogenous mouse loci provide non-human animals that express human IL1A or chimeric IL1A (e.g., humanized IL1A) in appropriate cell types and in a manner that does not result in the potential pathologies observed in some other transgenic mice known in the art. The human IL1A or the chimeric IL1A (e.g., humanized IL1A) expressed in animal can maintain one or more functions of the wild-type mouse or human IL1A in the animal. For example, human or non-human IL1A receptors (e.g., IL1R1) can bind to the expressed IL1A, and trigger an inflammatory cascade. Furthermore, in some embodiments, the animal does not express endogenous IL1A. As used herein, the term “endogenous IL1A” refers to IL1A protein that is expressed from an endogenous IL1A nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL1B (e.g., NP_000567.1 (SEQ ID NO: 4)). In some embodiments, the genome comprises a sequence encoding an amino acid sequence that is at least 70%, 75%, 80%, 85%, 90%, 95%, 99%, or 100% identical to SEQ ID NO: 4. The genome of the animal can comprise a sequence encoding an amino acid sequence that is at least 70%, 75%, 80%, 85%, 90%, 95%, 99%, or 100% identical to human IL1A (e.g., NP_000566.3 (SEQ ID NO: 11)). In some embodiments, the genome comprises a sequence encoding an amino acid sequence that is at least 70%, 75%, 80%, 85%, 90%, 95%, 99%, or 100% identical to SEQ ID NO: 11.

The genome of the genetically modified animal can comprise a replacement at an endogenous IL1B gene locus of a sequence encoding a region of endogenous IL1B with a sequence encoding a corresponding region of human IL1B. In some embodiments, the sequence that is replaced is any sequence within the endogenous IL1B gene locus, e.g., exon 1, exon 2, exon 3, exon 4, exon 5, exon 6, exon 7, 5′-UTR, 3′-UTR, the first intron, the second intron, and the third intron, the fourth intron, the fifth intron, the sixth intron, etc. In some embodiments, the sequence that is replaced is within the regulatory region of the endogenous IL1B gene. In some embodiments, the sequence that is replaced is exon 2, exon 3, exon 4, exon 5, exon 6, exon 7, or a part thereof, of an endogenous mouse IL1B gene locus. In some embodiments, the sequence that is replaced is starts within exon 2 and ends within exon 7 of an endogenous mouse IL1B gene locus. In some embodiments, the sequence that is replaced is from exon 2 to exon 7 of an endogenous mouse IL1B gene locus. In some embodiments, the coding region (starting from the “A” of start codon ATG and ending at the second “A” of stop codon TAA) of endogenous mouse IL1B gene is replaced.

The genome of the genetically modified animal can comprise a replacement at an endogenous IL1A gene locus of a sequence encoding a region of endogenous IL1A with a sequence encoding a corresponding region of human IL1A. In some embodiments, the sequence that is replaced is any sequence within the endogenous IL1A gene locus, e.g., exon 1, exon 2, exon 3, exon 4, exon 5, exon 6, exon 7, 5′-UTR, 3′-UTR, the first intron, the second intron, and the third intron, the fourth intron, the fifth intron, the sixth intron, etc. In some embodiments, the sequence that is replaced is within the regulatory region of the endogenous IL1A gene. In some embodiments, the sequence that is replaced is exon 2, exon 3, exon 4, exon 5, exon 6, exon 7, or a part thereof, of an endogenous mouse IL1A gene locus. In some embodiments, the sequence that is replaced is starts within exon 2 and ends within exon 7 of an endogenous mouse IL1A gene locus. In some embodiments, the sequence that is replaced is from exon 2 to exon 7 of an endogenous mouse IL1A gene locus. In some embodiments, the coding region (starting from the “A” of start codon ATG and ending at the second “A” of stop codon TAA) of endogenous mouse IL1A gene is replaced.

In some embodiments, the genetically modified animal does not express endogenous IL1B. In some embodiments, the genetically modified animal expresses a decreased level of endogenous IL1B as compared to a wild-type animal. In some embodiments, the genetically modified animal does not express endogenous IL1A. In some embodiments, the genetically modified animal expresses a decreased level of endogenous IL1A as compared to a wild-type animal.

Furthermore, the genetically modified animal can be heterozygous with respect to the replacement at the endogenous IL1B locus, or homozygous with respect to the replacement at the endogenous IL1B locus. Furthermore, the genetically modified animal can be heterozygous with respect to the replacement at the endogenous IL1A locus, or homozygous with respect to the replacement at the endogenous IL1A locus.

In some embodiments, the humanized IL1B locus lacks a human IL1B 5′-UTR. In some embodiment, the humanized IL1B locus comprises a rodent (e.g., mouse) 5′-UTR. In some embodiments, the humanization comprises a human 3′-UTR. In some embodiments, the humanization comprises a mouse 3′-UTR. In appropriate cases, it may be reasonable to presume that the mouse and human IL1B genes appear to be similarly regulated based on the similarity of their 5′-flanking sequence. As shown in the present disclosure, humanized IL1B mice that comprise a replacement at an endogenous mouse IL1B locus, which retain mouse regulatory elements but comprise a humanization of IL1B encoding sequence, do not exhibit pathologies. Both genetically modified mice that are heterozygous or homozygous for humanized IL1B are grossly normal.

In some embodiments, the humanized IL1A locus lacks a human IL1A 5′-UTR. In some embodiment, the humanized IL1A locus comprises a rodent (e.g., mouse) 5′-UTR. In some embodiments, the humanization comprises a human 3′-UTR. In some embodiments, the humanization comprises a mouse 3′-UTR. In appropriate cases, it may be reasonable to presume that the mouse and human IL1A genes appear to be similarly regulated based on the similarity of their 5′-flanking sequence. As shown in the present disclosure, humanized IL1A mice that comprise a replacement at an endogenous mouse IL1A locus, which retain mouse regulatory elements but comprise a humanization of IL1A encoding sequence, do not exhibit pathologies. Both genetically modified mice that are heterozygous or homozygous for humanized IL1A are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized IL1B gene. In some embodiments, the non-human mammal expresses a protein encoded by a humanized IL1A gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL1B in the genome of the mammal. The present disclosure also provides non-human mammals produced by any of the methods described herein. In some embodiments, a non-human mammal is provided; and the genetically modified animal contains the DNA encoding human or humanized IL1A in the genome of the mammal.

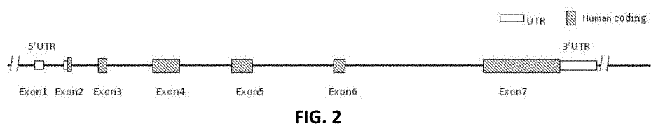

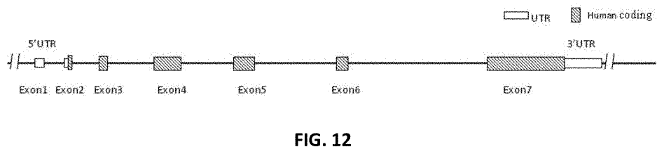

In some embodiments, the non-human mammal comprises the genetic construct as described herein (e.g., gene construct as shown in FIG. 2 or FIG. 12). In some embodiments, a non-human mammal expressing human or humanized IL1B is provided. In some embodiments, a non-human mammal expressing human or humanized IL1A is provided. In some embodiments, the tissue-specific expression of human or humanized IL1B protein is provided. In some embodiments, the tissue-specific expression of human or humanized IL1A protein is provided.

In some embodiments, the expression of human or humanized IL1B in a genetically modified animal is controllable, as by the addition of a specific inducer or repressor substance. In some embodiments, the expression of human or humanized IL1A in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can be performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human IL1B and/or IL1A protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL1B and/or IL1A protein.

### Vectors

In one aspect, the present disclosure relates to a targeting vector, comprising: a) a DNA fragment homologous to the 5′ end of a region to be altered (5′ arm), which is selected from the IL1B gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL1B gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC 000068.7; c) the DNA fragment homologous to the 3′ end of the region to be altered (3′ arm) is selected from the nucleotide sequences that have at least 90% homology to the NCBI accession number NC_000068.7.

In some embodiments, a) the DNA fragment homologous to the 5′ end of a region to be altered (5′ arm) is selected from the nucleotides from the position 129370331 to the position 129375271 of the NCBI accession number NC_000068.7; c) the DNA fragment homologous to the 3′ end of the region to be altered (3′ arm) is selected from the nucleotides from the position 129360159 to the position 129364160 of the NCBI accession number NC_000068.7.

In some embodiments, the length of the selected genomic nucleotide sequence in the targeting vector can be more than about 1 kb, about 1.5 kb, about 2 kb, about 2.5 kb, 3 kb, about 3.5 kb, about 4 kb, about 4.5 kb, about 5 kb, about 5.5 kb, or about 6 kb.

In some embodiments, the region to be altered is exon 1, exon 2, exon 3, exon 4, exon 5, exon 6, and/or exon 7 of IL1B gene (e.g., a portion of exon 2, exon 3, exon 4, exon 5, exon 6, and a portion of exon 7 of mouse IL1B gene).

The targeting vector can further include a selected gene marker.

In some embodiments, the sequence of the 5′ arm is shown in SEQ ID NO: 6; and the sequence of the 3′ arm is shown in SEQ ID NO: 7.

In some embodiments, the sequence is derived from human (e.g., 112830361-112836229 of NC_000002.12). For example, the target region in the targeting vector is a part or entirety of the nucleotide sequence of a human IL1B, preferably comprising exon 2, exon 3, exon 4, exon 5, exon 6, and/or exon 7, or a part thereof, of the human IL1B. In some embodiments, the nucleotide sequence of the humanized IL1B encodes the entire or the part of human IL1B protein with the NCBI accession number NP_000567.1 (SEQ ID NO: 4).

In one aspect, the present disclosure relates to a targeting vector, comprising: a) a DNA fragment homologous to the 5′ end of a region to be altered (5′ arm), which is selected from the IL1A gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL1A gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000068.7; c) the DNA fragment homologous to the 3′ end of the region to be altered (3′ arm) is selected from the nucleotide sequences that have at least 90% homology to the NCBI accession number NC_000068.7.

In some embodiments, a) the DNA fragment homologous to the 5′ end of a region to be altered (5′ arm) is selected from the nucleotides from the position 129309102 to the position 129313901 of the NCBI accession number NC_000068.7; c) the DNA fragment homologous to the 3′ end of the region to be altered (3′ arm) is selected from the nucleotides from the position 129295411 to the position 129299309 of the NCBI accession number NC_000068.7.

In some embodiments, the length of the selected genomic nucleotide sequence in the targeting vector can be more than about 1 kb, about 1.5 kb, about 2 kb, about 2.5 kb, 3 kb, about 3.5 kb, about 4 kb, about 4.5 kb, about 5 kb, about 5.5 kb, about 6 kb, about 6.5 kg, about 7 kb, about 7.5 kb, about 8 kb, about 8.5 kb, or about 9 kb.

In some embodiments, the region to be altered is exon 1, exon 2, exon 3, exon 4, exon 5, exon 6, and/or exon 7 of IL1A gene (e.g., a portion of exon 2, exon 3, exon 4, exon 5, exon 6, and a portion of exon 7 of mouse IL1A gene).

The targeting vector can further include a selected gene marker.

In some embodiments, the sequence of the 5′ arm is shown in SEQ ID NO: 13; and the sequence of the 3′ arm is shown in SEQ ID NO: 14.

In some embodiments, the sequence is derived from human (e.g., 112775067-112783770 of NC_000002.12). For example, the target region in the targeting vector is a part or entirety of the nucleotide sequence of a human IL1A, preferably comprising exon 2, exon 3, exon 4, exon 5, exon 6, and/or exon 7, or a part thereof, of the human IL1A. In some embodiments, the nucleotide sequence of the humanized IL1A encodes the entire or the part of human IL1A protein with the NCBI accession number NP_000566.3 (SEQ ID NO: 11).

The disclosure also relates to a cell comprising the targeting vectors as described above. In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., non-homologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL1B gene locus, a sequence encoding a region of an endogenous IL1B with a sequence encoding a corresponding region of human or chimeric IL1B. In some embodiments, the disclosure provides replacing in at least one cell of the animal, at an endogenous IL1A gene locus, a sequence encoding a region of an endogenous IL1A with a sequence encoding a corresponding region of human or chimeric IL1A. In some embodiments, the replacement occurs in a germ cell, a somatic cell, a blastocyst, or a fibroblast, etc. The nucleus of a somatic cell or the fibroblast can be inserted into an enucleated oocyte.

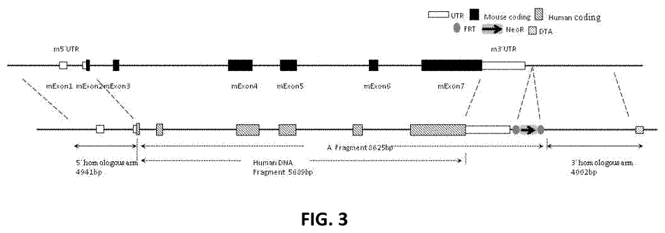

FIG. 3 shows a humanization strategy for a mouse IL1B locus. In FIG. 3, the targeting strategy involves a vector comprising the 5′ end homologous arm, human IL1B gene fragment, 3′ homologous arm. The process can involve replacing endogenous IL1B sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL1B sequence with human IL1B sequence.

Thus, in some embodiments, the methods for making a genetically modified, humanized animal, can include the step of replacing at an endogenous IL1B locus (or site), a nucleic acid encoding a sequence encoding a region of endogenous IL1B with a sequence encoding a corresponding region of human IL1B. The sequence can include a region (e.g., a part or the entire region) of exon 1, exon 2, exon 3, exon 4, exon 5, exon 6, and/or exon 7 of an endogenous or human IL1B gene. In some embodiments, the sequence includes a region of exon 2, exon 3, exon 4, exon 5, exon 6, and a region of exon 7 of a human IL1B gene (e.g., a sequence encoding amino acids 1-269 of SEQ ID NO: 4). In some embodiments, the endogenous IL1B locus is exon 2, exon 3, exon 4, exon 5, exon 6, and/or exon 7 of mouse IL1B gene (e.g., a sequence encoding amino acids 1-269 of SEQ ID NO: 2).

In some embodiments, the methods of modifying a IL1B locus of a mouse to express a chimeric human/mouse IL1B peptide can include the steps of replacing at the endogenous mouse IL1B locus a nucleotide sequence encoding a mouse IL1B with a nucleotide sequence encoding a human IL1B, thereby generating a sequence encoding a chimeric human/mouse IL1B.

In some embodiments, provided herein is a genetically-modified non-human animal whose genome comprises a sequence that is at least 70%, 75%, 80%, 85%, 90%, 95%, 99%, or 100% identical to SEQ ID NO: 15, 16, 17, or 18.

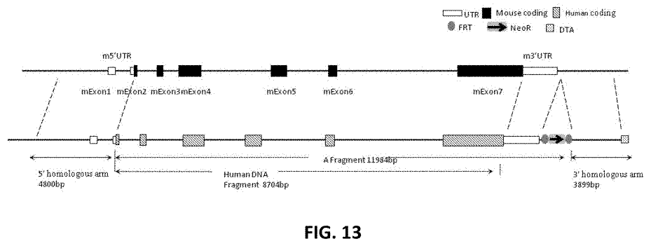

FIG. 13 shows a humanization strategy for a mouse IL1A locus. In FIG. 13, the targeting strategy involves a vector comprising the 5′ end homologous arm, human IL1A gene fragment, 3′ homologous arm. The process can involve replacing endogenous IL1A sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL1A sequence with human IL1A sequence.

Thus, in some embodiments, the methods for making a genetically modified, humanized animal, can include the step of replacing at an endogenous IL1A locus (or site), a nucleic acid encoding a sequence encoding a region of endogenous IL1A with a sequence encoding a corresponding region of human IL1A. The sequence can include a region (e.g., a part or the entire region) of exon 1, exon 2, exon 3, exon 4, exon 5, exon 6, and/or exon 7 of an endogenous or human IL1A gene. In some embodiments, the sequence includes a region of exon 2, exon 3, exon 4, exon 5, exon 6, and a region of exon 7 of a human IL1A gene (e.g., a sequence encoding amino acids 1-271 of SEQ ID NO: 11). In some embodiments, the endogenous IL1A locus is exon 2, exon 3, exon 4, exon 5, exon 6, and/or exon 7 of mouse IL1A gene (e.g., a sequence encoding amino acids 1-270 of SEQ ID NO: 9).

In some embodiments, the methods of modifying a IL1A locus of a mouse to express a chimeric human/mouse IL1A peptide can include the steps of replacing at the endogenous mouse IL1A locus a nucleotide sequence encoding a mouse IL1A with a nucleotide sequence encoding a human IL1A, thereby generating a sequence encoding a chimeric human/mouse IL1A.

In some embodiments, provided herein is a genetically-modified non-human animal whose genome comprises a sequence that is at least 70%, 75%, 80%, 85%, 90%, 95%, 99%, or 100% identical to SEQ ID NO: 32, 33, 34, or 35.

In some embodiments, the nucleotide sequences as described herein do not overlap with each other (e.g., the 5′ homologous arm, the “A fragment”, and/or the 3′ homologous arm do not overlap). In some embodiments, the amino acid sequences as described herein do not overlap with each other.

The present disclosure further provides a method for establishing a IL1B and/or IL1A gene humanized animal model, involving the following steps:

(a) providing the cell (e.g. an embryonic stem cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the embryonic stem cells for the methods described above are C57BL/6 embryonic stem cells. Other embryonic stem cells that can also be used in the methods as described herein include, but are not limited to, FVB/N embryonic stem cells, BALB/c embryonic stem cells, DBA/1 embryonic stem cells and DBA/2 embryonic stem cells.

Embryonic stem cells can come from any non-human animal, e.g., any non-human animal as described herein. In some embodiments, the embryonic stem cells are derived from rodents. The genetic construct can be introduced into an embryonic stem cell by microinjection of DNA. For example, by way of culturing an embryonic stem cell after microinjection, a cultured embryonic stem cell can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized IL1B and/or IL1A protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized IL1B, which are useful for testing agents that can decrease or block the interaction between IL1B and IL1B receptors (e.g., IL1R1) or the interaction between IL1B and anti-human IL1B antibodies, testing whether an agent can increase or decrease the immune response, and/or determining whether an agent is an IL1B agonist or antagonist. In various aspects, genetically modified animals are provided that express human or humanized IL1A, which are useful for testing agents that can decrease or block the interaction between IL1A and IL1A receptors (e.g., IL1R1) or the interaction between IL1A and anti-human IL1A antibodies, testing whether an agent can increase or decrease the immune response, and/or determining whether an agent is an agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In one aspect, the disclosure also provides methods of determining effectiveness of an IL1B antagonist (e.g., an anti-IL1B antibody) for reducing inflammation. The methods involve administering the IL1B antagonist to the animal described herein, wherein the animal has an inflammation; and determining effects of the IL1B antagonist for reducing the inflammation. In one aspect, the disclosure also provides methods of determining effectiveness of an IL1A antagonist (e.g., an anti-IL1A antibody) for reducing inflammation. The methods involve administering the IL1A antagonist to the animal described herein, wherein the animal has an inflammation; and determining effects of the IL1A antagonist for reducing the inflammation.

In one aspect, the disclosure also provides methods of determining effectiveness of an IL1B antagonist (e.g., an anti-IL1B antibody) for treating an immune disorder (e.g., an autoimmune disorder or allergic disorder). The methods involve administering the IL1B antagonist to the animal described herein, wherein the animal has an immune disorder; and determining effects of the IL1B antagonist for treating the immune disorder. In one aspect, the disclosure also provides methods of determining effectiveness of an IL1A antagonist (e.g., an anti-IL1A antibody) for treating an immune disorder (e.g., an autoimmune disorder or allergic disorder). The methods involve administering the IL1A antagonist to the animal described herein, wherein the animal has an immune disorder; and determining effects of the IL1A antagonist for treating the immune disorder.

In one aspect, the disclosure also provides methods of determining effectiveness of an agent for treating autoimmune disorder. The methods involve administering the agent to the animal described herein, wherein the animal has an autoimmune disorder; and determining effects of the agent for treating the autoimmune disorder. In some embodiments, the autoimmune disorder is psoriasis. In some embodiments, psoriasis is induced, e.g., by applying an immune response modifier (e.g., 5% imiquimod cream) to the skin of the animal (e.g., mouse). In some embodiments, the immune response modifier induces local inflammatory effects of the skin. In some embodiments, the skin is shaved before applying the immune response modifier. In some embodiments, the agent is a steroid or corticosteroid, e.g., bethamethasone, prednisone, prednisolone, triamcinolone, methylprednisolone, or dexamethasone. In some embodiments, the agent is hydrocortisone, calamine lotion, camphor, or benzocaine. In some embodiments, the agent is an anti-IL1B or anti-IL1A antibody. In some embodiments, the agent is a non-steroidal anti-inflammatory drug, disease-modifying antirheumatic drug, or immunosuppressant. In some embodiments, the effects are evaluated by clinical scores (e.g., Psoriasis Area Severity Index to measure the severity and extent of psoriasis). In some embodiments, the effects are evaluated by staining the relevant skin tissues, e.g., by hematoxylin and eosin (HE) staining. Details of imiquimod-induced psoriasis model can be found, e.g., in Sakai, Kent, et al. “Mouse model of imiquimod-induced psoriatic itch.” Pain 157.11 (2016): 2536, which is incorporated herein by reference in its entirety.

In some embodiments, the genetically modified animals can be used for determining effectiveness of an anti-IL1B antibody for treating cancer. The methods involve administering the anti-IL1B antibody (e.g., anti-human IL1B antibody) to the animal as described herein, wherein the animal has a tumor; and determining the inhibitory effects of the anti-IL1B antibody to the tumor. In some embodiments, the genetically modified animals can be used for determining effectiveness of an anti-IL1A antibody for treating cancer. The methods involve administering the anti-IL1A antibody (e.g., anti-human IL1A antibody) to the animal as described herein, wherein the animal has a tumor; and determining the inhibitory effects of the anti-IL1A antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the IL1B antibody is a monoclonal antibody. In some embodiments, the IL1B antibody is Gevokizumab. Details of Gevokizumab can be found, e.g., in WO2007002261A2, which is incorporated herein by reference in its entirety. In some embodiments, the IL1B antibody is Canakinumab (ACZ885, or Ilaris®. Details of Antibody 43 can be found, e.g., in WO2002016436A2, which is incorporated herein by reference in its entirety.

In some embodiments, the tumor comprises one or more cancer cells (e.g., human or mouse cancer cells) that are injected into the animal. In some embodiments, the anti-IL1B antibody prevents IL1R1 from binding to IL1B. In some embodiments, the anti-IL1B antibody does not prevent IL1R1 from binding to IL1B. In some embodiments, the anti-IL1A antibody prevents IL1R1 from binding to IL1A. In some embodiments, the anti-IL1A antibody does not prevent IL1R1 from binding to IL1A.

In some embodiments, the genetically modified animals can be used for determining whether an anti-IL1B antibody is a IL1B agonist or antagonist. In some embodiments, the methods as described herein are also designed to determine the effects of an agent (e.g., a steroid (e.g., dexamethasone), or anti-IL1B antibodies) on IL1B, e.g., reducing inflammation. In some embodiments, the genetically modified animals can be used for determining whether an anti-IL1A antibody is a IL1A agonist or antagonist. In some embodiments, the methods as described herein are also designed to determine the effects of an agent (e.g., a steroid (e.g., dexamethasone), or anti-IL1A antibodies) on IL1A, e.g., reducing inflammation. In some embodiments, the genetically modified animals can be used for determining the effective dosage of a therapeutic agent for treating a disease in the subject, e.g., an immune disorder, an allergy, or autoimmune diseases (e.g., psoriasis).

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVₜ/TV_{c})×100, where TVt and TVc are the mean tumor volume (or weight) of treated and control groups.

In some embodiments, the anti-IL1B antibody or the anti-IL1A antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the anti-IL1B antibody or the anti-IL1A antibody is designed for treating breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, urothelial cancer, oral cancer, or bone cancer. In some embodiments, the anti-IL1B or anti-IL1A antibody is designed for treating solid tumor. In some embodiments, the anti-IL1B or anti-IL1A antibody is designed for treating metastatic solid tumors. In some embodiments, the anti-IL1B or anti-IL1A antibody is designed for reducing tumor growth, metastasis, and/or angiogenesis. In some embodiments, the anti-IL1B or anti-IL1A antibody is designed for treating hematopoietic malignancies.

In some embodiments, the cancer types as described herein include, but not limited to, lymphoma, non-small cell lung cancer (NSCLC), leukemia, ovarian cancer, nasopharyngeal cancer, breast cancer, endometrial cancer, colon cancer, rectal cancer, stomach cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, and sarcoma. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T cell lymphoma, and Waldenstrom macroglobulinemia. In some embodiments, the sarcoma is selected from osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma.

In some embodiments, the antibody is designed for treating various autoimmune diseases or allergy (e.g., psoriasis, allergic rhinitis, sinusitis, asthma, rheumatoid arthritis, atopic dermatitis, chronic obstructive pulmonary disease (COPD), chronic bronchitis, emphysema, eczema, osteoarthritis, rheumatoid arthritis, systemic lupus erythematosus, polymyalgia rheumatica, autoimmune hemolytic anemia, systemic vasculitis, pernicious anemia, inflammatory bowel disease, ulcerative colitis, Crohn's disease, or multiple sclerosis). Thus, the methods as described herein can be used to determine the effectiveness of an antibody in inhibiting immune response.

In some embodiments, the immune disorder or immune-related diseases described here include allergy, asthma, myocarditis, nephritis, hepatitis, systemic lupus erythematosus, rheumatoid arthritis, scleroderma, hyperthyroidism, primary thrombocytopenic purpura, autoimmune hemolytic anemia, ulcerative colitis, self-immune liver disease, diabetes, pain, or neurological disorders.

In some embodiments, the antibodies is designed for treating various acute or chronic autoinflammatory diseases (e.g., familial Mediterranean fever, familial cold-induced autoinflammatory syndrome, cryopyrin-associated periodic syndrome (CAPS), Hyper IgD Syndrome, Adult and Juvenile Still's Disease, Behcet's Disease, Schnitzler's Syndrome, TNF Receptor-Associated Periodic Syndrome, PAPA Syndrome, Blau's Syndrome, Sweet's Syndrome, Urticarial Vasculitis, Anti-synthetase Syndrome, Recurrent Idiopathic Pericarditis, Relapsing Perichondritis, Urate Crystal Arthritis (gout), Type-2 Diabetes, Smoldering Multiple Myeloma, Post-myocardial Infarction Heart Failure, or Osteoarthritis.

In some embodiments, the antibody is designed for reducing inflammation (e.g., inflammatory bowel disease, chronic inflammation, asthmatic inflammation, periodontitis, or wound healing). Thus, the methods as described herein can be used to determine the effectiveness of an antibody for reducing inflammation. In some embodiments, the inflammation described herein includes degenerative inflammation, exudative inflammation, serous inflammation, fibrinitis, suppurative inflammation, hemorrhagic inflammation, necrotitis, catarrhal inflammation, proliferative inflammation, specific inflammation, tuberculosis, syphilis, leprosy, or lymphogranuloma. In some embodiments, the inflammation is cryopyrin-associated periodic syndrome (CAPS). In some embodiments, the inflammation is a skin disease, e.g., acne.

In some embodiments, the antibody is designed for treating other diseases (e.g., endometriosis).

The present disclosure also provides methods of determining toxicity of an antibody (e.g., anti-IL1B or anti-IL1A antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL1B gene function, human IL1B antibodies, drugs for human IL1B targeting sites, the drugs or efficacies for human IL1B targeting sites, the drugs for immune-related diseases and antitumor drugs. The disclosure also relates to the use of the animal model generated through the methods as described herein in the screening, verifying, evaluating or studying the IL1A gene function, human IL1A antibodies, drugs for human IL1A targeting sites, the drugs or efficacies for human IL1A targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the IL1B and/or IL1A gene humanized non-human animal prepared by the methods described herein, the IL1B and/or IL1A gene humanized non-human animal described herein, the double- or multi-humanized non-human animal generated by the methods described herein (or progeny thereof), a non-human animal expressing the human or humanized IL1B and/or IL1A protein, or the tumor-bearing or inflammatory animal models described herein. In some embodiments, the TCR-T, CAR-T, and/or other immunotherapies can treat the IL1B-associated or IL1A-associated diseases described herein. In some embodiments, the TCA-T, CAR-T, and/or other immunotherapies provides an evaluation method for treating the IL1B-associated or IL1A-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric IL1B and/or IL1A gene and a sequence encoding an additional human or chimeric protein.

In some embodiments, the additional human or chimeric protein can be interleukin 1 alpha (IL1A), interleukin 1 beta (IL1B), IL-1 receptor type I (IL1R1), interleukin-1 receptor accessory protein (IL1RAP), programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

The methods of generating genetically modified animal model with two or more human or chimeric genes (e.g., humanized genes) can include the following steps:

(a) using the methods of introducing human IL1B gene (or human IL1A gene) or chimeric IL1B gene (or chimeric IL1A gene) as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL1A, IL1B, IL1R1, IL1RAP, PD-1, CTLA-4, LAG-3, IL15 receptor, BTLA, PD-L1, CD3, CD27, CD28, CD47, CD137, CD154, TIGIT, TIM-3, GITR, SIRPα or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the IL1B and/or IL1A humanization is directly performed on a genetically modified animal having a human or chimeric IL1R1, IL1RAP, PD-1, CTLA-4, LAG-3, IL15 receptor, BTLA, PD-L1, CD3, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL1B or anti-IL1A antibody and an additional therapeutic agent for the treatment of cancer or an immune disorder. The methods include administering the anti-IL1B or anti-IL1A antibody and the additional therapeutic agent to the animal, wherein the animal has a tumor; and determining the inhibitory effects of the combined treatment to the tumor. In some embodiments, the additional therapeutic agent is an antibody that specifically binds to PD-1, CTLA-4, LAG-3, IL15 receptor, BTLA, PD-L1, CD3, CD27, CD28, CD47, CD137, CD154, TIGIT, TIM-3, GITR, SIRPα or OX40. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, urothelial cancer, oral cancer, or bone cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

SspI, SpeI, AseI, NcoI, and DraIII restriction enzymes were purchased from NEB with catalog numbers: R0132, R3133V, R0526V, R3193V, and R3510V, respectively.

Lipopolysaccharides from Escherichia coli O111:B4 was purchased from Sigma with catalog number L2630.

Mouse IL-1β ELISA MAX™ Deluxe kit was purchased from BioLegend with catalog number 432604.

ELISA MAX™ Deluxe Set Human IL-1β kit was purchased from BioLegend with catalog number 437004.

ELISA MAX™ Deluxe Set Mouse IL-la kit was purchased from BioLegend with catalog number 433404.

LEGEND MAX™ Human IL-la ELISA kit was purchased from BioLegend with catalog number 445807.

Attune™ Nxt Acoustic Focusing Cytometer was purchased from Thermo Fisher Scientific (Model: Attune™ Nxt).

PrimeScript™ 1st Strand cDNA Synthesis Kit was purchased from Takara Bio Inc. with catalog number 6110A.

Heraeus™ Fresco™ 21 Microcentrifuge was purchased from Thermo Fisher Scientific (Model: Fresco™ 21).

### Example 1: Mice with Humanized IL1B Gene

A gene sequence encoding the human IL1B protein can be introduced into the endogenous mouse IL1B locus, such that the mouse can express a human or humanized IL1B protein. The mouse IL1B gene (NCBI Gene ID: 16176, Primary source: MGI: 96543, UniProt ID: P10749) comprises 7 exons, and is located at 129364569 to 129371164 of chromosome 2 (NC_000068.7). The human IL1B gene (NCBI Gene ID: 3553, Primary source: HGNC: 5992, UniProt ID: P01584) comprises 7 exons, and is located at 112829751 to 112836843 of chromosome 2 (NC_000002.12). The mouse IL1B transcript sequence NM 008361.4 is set forth in SEQ ID NO: 1, and the corresponding protein sequence NP_032387.1 is set forth in SEQ ID NO: 2. The human IL1B transcript sequence NM 000576.3 is set forth in SEQ ID NO: 3, and the corresponding protein sequence NP_000567.1 is set forth in SEQ ID NO: 4. Mouse and human IL1B gene loci are shown in FIG. 1A and FIG. 1B, respectively.

Mouse cells can be modified by various gene-editing techniques, for example, replacement of specific mouse IL1B gene sequences with human IL1B gene sequences at the endogenous mouse IL1B locus. For example, a sequence starting within exon 2 (from start codon ATG) and ending within exon 7 (to stop codon TAA) of mouse IL1B gene was replaced with a sequence starting within exon 2 (from start codon ATG) and ending within exon 7 (to stop codon TAA) of human IL1B gene to obtain a humanized IL1B locus, thereby humanizing mouse IL1B gene (shown in FIG. 2).

As shown in the schematic diagram of the targeting strategy in FIG. 3, the targeting vector contained homologous arm sequences upstream and downstream of mouse IL1B gene locus, and an “A fragment” comprising a human IL1B gene sequence. The upstream homologous arm sequence (5′ homologous arm, SEQ ID NO: 6, 4941 bp) is identical to nucleotide sequence of 129370331-129375271 of NCBI accession number NC_000068.7, and the downstream homologous arm sequence (3′ homologous arm, SEQ ID NO: 7, 3458 bp) is identical to nucleotide sequence of 129360159-129364160 of NCBI accession number NC_000068.7. The A fragment comprises a genomic DNA sequence of 5869 bp (SEQ ID NO: 5) from a portion of exon 2 (starting from start codon ATG) to a portion of exon 7 (ending at stop codon TAA) of human IL1B gene, which is identical to nucleotide sequence of 112830361-112836229 of NCBI accession number NC_000002.12. The connection between the upstream of the human DNA fragment in the “A fragment” and the mouse sequence is designed as: ACTTTCTTTCTTCACACAGGTGTCTGAAGCAGCTATGGCAGAAGTACCTGAGCTCGC CAGTGAAATGATGGCTTATTACAG (SEQ ID NO: 15), wherein the “T” in sequence “AGCT” is the last nucleotide of the mouse sequence, and the “A” in sequence “ATGG” is the first nucleotide of the human sequence. The connection between the downstream of the human DNA fragment and the mouse sequence is designed as: CCATGCAATTTGTGTCTTCCTAAAGTATGGGCTGGACTGTTTCTAATGCCTTCCCCAG GGC (SEQ ID NO: 16), wherein the second “A” in sequence “TAA” is the last nucleotide of the human sequence, and the “A” in sequence “AGT” is the first nucleotide of the mouse sequence.

The targeting vector also included an antibiotic resistance gene for positive clone screening (neomycin phosphotransferase gene, or Neo), and two Frt recombination sites flanking the antibiotic resistance gene, that formed a Neo cassette. The connection between the upstream of the Neo cassette and the mouse sequence is designed as: TTTGGCAACAGGAAGATCTCTGGGCTTGACAGCAGCCATCTACTAGGGTTAACGAAT TCCGAAGTTCCTATTCTCTAGAAAGTATAGGAACTTCAGGTCTGAAGA (SEQ ID NO: 17), wherein the second “G” in sequence “TAGG” is the last nucleotide of the mouse sequence, and the “G” in sequence “GTTA” is the first nucleotide of the Neo cassette. The downstream connection of the Neo cassette is designed as: CTTCGAAGTTCCTATTCTCTAGAAAGTATAGGAACTTCATCAGTCAGGTACATAATT AGGTGGATCCAATATTGATATCGGTACCTATCCACATCTAGCAAGGAACCCTGTCTC AAAAATTAAGGTGGAAAATAATTGAGGAAGACAACTGATTTTGATCTCTGGTCTCC (SEQ ID NO: 18), wherein the second “C” in sequence “TACC” is the last nucleotide of the Neo cassette, and the first T in sequence “TATC” is the first nucleotide of the mouse sequence. In addition, a coding gene with a negative selectable marker (a gene encoding diphtheria toxin A subunit (DTA)) was also inserted downstream of the 3′ homologous arm of the targeting vector. The modified humanized mouse IL1B mRNA sequence is shown as nucleic acids 88-897 of SEQ ID NO: 3, and the expressed protein has the same sequence as human IL1B protein shown in SEQ ID NO: 4.

The targeting vector was constructed, e.g., by restriction enzyme digestion and ligation. The constructed targeting vector sequence was preliminarily verified by restriction enzyme digestion, followed by verification by sequencing. The correct targeting vector was electroporated and transfected into embryonic stem cells of C57BL/6 mice. The positive selectable marker gene was used to screen the cells, and the integration of exogenous genes was confirmed by PCR and Southern Blot.

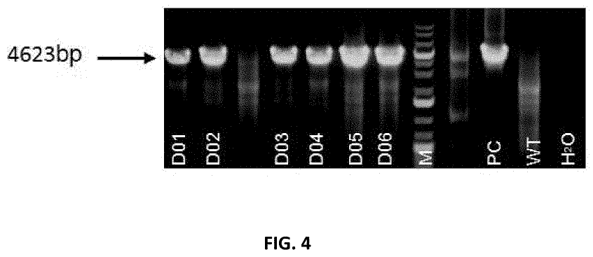

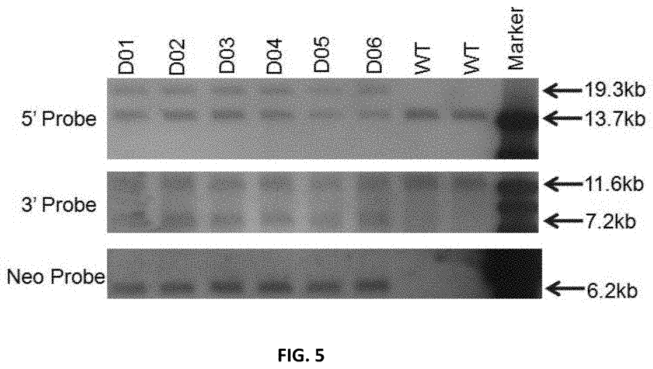

Specifically, PCR primers ES-F and ES-R were used for amplification. The results are shown in FIG. 4. All 6 clones, i.e., D01, D02, D03, D04, D05, and D06, were identified as positive clones. The positive clones identified by PCR were further confirmed by Southern Blot (digested with SpeI, SspI, or AseI, respectively, and hybridized with 3 probes) to screen out correct positive clone cells. The length of the probes and the size of target fragments are shown in Table 5, and the results are shown in FIG. 5. All 6 clones confirmed by PCR and Southern Blot, were further verified by sequencing and no random insertions were detected.

TABLE 5
IL1B gene detection probe and target fragment size

| Restriction enzyme | Probe | Wild-type | Recombinant sequence | |
|---|---|---|---|---|
| SpeI | 5′ Probe | 13.7 kb | 19.3 | kb |
| SpeI | 3′ Probe | 11.6 kb | 7.2 | kb |
| AseI | Neo Probe | — | 6.2 | kb |

The following primers were used in PCR:

ES-F:
(SEQ ID NO: 19)
5′-CAGGACATAGCGTTGGCTAC-3′
ES-R:
(SEQ ID NO: 20)
5′-TTAGCCAACAGGCTACAGAACCACG-3′

The following probes were used in Southern Blot assays:

5′ Probe:
5′ Probe-F:
(SEQ ID NO: 21)
5′-CATCCATAACCAAGGCTGCCAGTCA-3′
5′ Probe-R:
(SEQ ID NO: 22)
5′-AATTGCTCTGACCACTTACTGCCCC-3′
3′ Probe:
3′ Probe-F:
(SEQ ID NO: 23)
5′-CTTGTTCCTTGCTCTTCACCAGCCC-3′
3′ Probe-R:
(SEQ ID NO: 24)
5′-CGGCCAATGCATCTTCTGTGTTTCAA-3′
Neo Probe:
NeoProbe-F:
(SEQ ID NO: 25)
5′-GGATCGGCCATTGAACAAGAT-3′
NeoProbe-R:
(SEQ ID NO: 26)
5′-CAGAAGAACTCGTCAAGAAGGC-3′

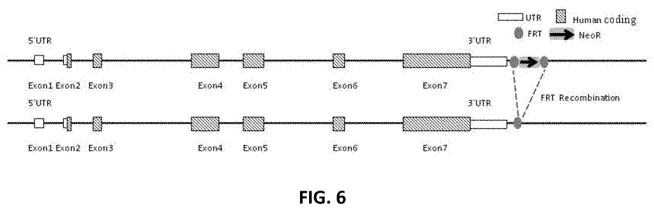

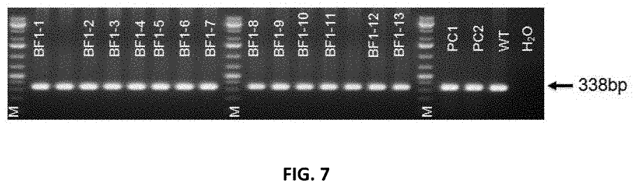

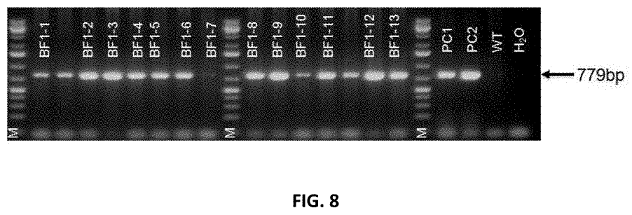

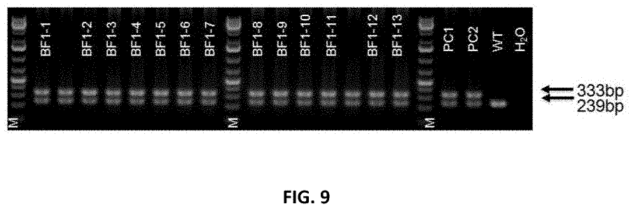

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp mice to remove the positive selectable marker gene (the process diagram is shown in FIG. 6), and then the humanized IL1B homozygous mice expressing human IL1B protein were obtained by breeding with each other. The genotype of the progeny mice can be identified by PCR using primers shown in Table 6. The identification results of exemplary F1 generation mice (Neo cassette-removed) are shown in FIGS. 7-9, and a total of 13 mice labelled BF1-1, BF1-2, BF1-3, BF1-4, BF1-5, BF1-6, BF1-7, BF1-8, BF1-9, BF1-10, BF1-11, BF1-12, and BF1-13 were identified as positive heterozygous clones.

TABLE 6
IL IB gene detection primer sequence

| | | | Fragment |
|---|---|---|---|
| | | Tm | size |
| Primer | Sequence (5+40-3′) | (° C.) | (bp) |
| ILIB-F | CTTCCTGGGAAACAACAGTGGTC | 60 | WT: 338 |
| | (SEQ ID NO: 27) | | |
| IL1B-R | GAGAGTGCTGCCTAATGTCCCCTTG | 64 | |
| | (SEQ ID NO: 28) | | |
| IL1B-F1 | GCCCCTGGAAACTAGGTACTTCAAG | 62 | Mut: 779 |
| | (SEQ ID NO: 29) | | |
| IL1B-R | GAGAGTGCTGCCTAATGTCCCCTTG | 64 | |
| | (SEQ ID NO: 28) | | |
| Frt-F | TGAGCAAGAGGACCTGAATGAG | 57 | WT: 239 |
| | (SEQ ID NO: 30) | | (Neo |
| | | | cassette- |
| | | | removed) |
| Frt-R | CATTCACATGTGTGTAGGGGTGGA | 59 | Mut: 333 |
| | (SEQ ID NO: 31) | | |

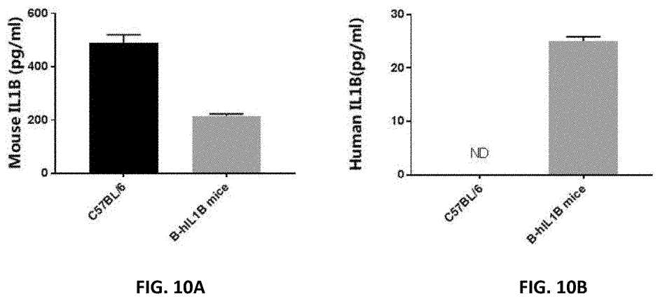

The results indicate that this method can be used to construct humanized IL1B genetically engineered mice that can be passaged stably without random insertion. The expression of human IL1B protein in positive mice can be confirmed, e.g., by enzyme-linked immunosorbent assay (ELISA). For example, BioLegend Mouse IL-1β ELISA MAX™ Deluxe Kit and BioLegend ELISA MAX™ Deluxe Set Human IL-1β Kit were used herein. The control group used C57BL/6 wild-type mice, and the experimental group used IL1B humanized heterozygous mice. Specifically, mouse bone marrow samples were collected to isolate monocytes, and 1 μg/mL Lipopolysaccharide (LPS) was used to stimulate the monocytes for 24 hours. Supernatant was collected for ELISA analysis. The test procedure was carried out according to with the instructions of the kits. As shown in FIGS. 10A-10B, expression of mouse IL1B protein was detected in both wild-type mice and IL1B humanized heterozygous mice. However, expression of human IL1B protein was only detected in IL1B humanized heterozygous mice. The results indicate that the humanized IL1B heterozygous mice can successfully express human IL1B protein in vivo.

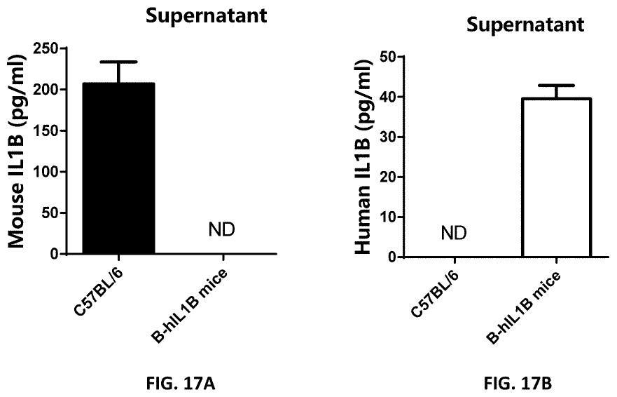

In another experiment, expression of human IL1B protein in the positive IL1B homozygous mice was confirmed by ELISA. Three female wild-type C57BL/6 mice and three female IL1B gene humanized homozygous mice were selected, and 1 μg Lipopolysaccharide (LPS) was injected to each mouse intraperitoneally. After 24 hours, mouse serum was collected and the expression of human IL1B protein was detected by the aforementioned ELISA detection method. As shown in FIGS. 17A-17B, expression of mouse IL1B protein was detected in wild-type C57BL/6 mice, but the expression of human IL1B protein was not detected; by contrast, expression of human IL1B protein was detected in IL1B humanized homozygous mice, but the expression of mouse IL1B protein was not detected.

### Example 2: Mice with Humanized IL1A Gene

A gene sequence encoding the human IL1A protein can be introduced into the endogenous mouse IL1A locus, such that the mouse can express a human or humanized IL1A protein. The mouse IL1A gene (NCBI Gene ID: 16175, Primary source: MGI: 96542, UniProt ID: P01582) comprises 7 exons, and is located at 129299609 to 129310186 of chromosome 2 (NC_000068.7). The human IL1A gene (NCBI Gene ID: 3552, Primary source: HGNC: 5991, UniProt ID: P01583) comprises 7 exons, and is located at 112773925 to 112784493 of chromosome 2 (NC_000002.12). The mouse IL1A transcript sequence NM 010554.4 is set forth in SEQ ID NO: 8, and the corresponding protein sequence NP_034684.2 is set forth in SEQ ID NO: 9. The human IL1A transcript sequence NM 000575.5 is set forth in SEQ ID NO: 10, and the corresponding protein sequence NP_000566.3 is set forth in SEQ ID NO: 11. Mouse and human IL1A gene loci are shown in FIG. 11A and FIG. 11B, respectively.

Mouse cells can be modified by various gene-editing techniques, for example, replacement of specific mouse IL1A gene sequences with human IL1A gene sequences at the endogenous mouse IL1A locus. For example, a sequence starting within exon 2 (from start codon ATG) and ending within exon 7 (to stop codon TAA) of mouse IL1A gene was replaced with a sequence starting within exon 2 (from start codon ATG) and ending within exon 7 (to stop codon TAA) of human IL1A gene to obtain a humanized IL1A locus, thereby humanizing mouse IL1A gene (shown in FIG. 12).

As shown in the schematic diagram of the targeting strategy in FIG. 13, the targeting vector contained homologous arm sequences upstream and downstream of mouse IL1A gene locus, and an “A fragment” comprising a human IL1A gene sequence. The upstream homologous arm sequence (5′ homologous arm, SEQ ID NO: 13, 4800 bp) is identical to nucleotide sequence of 129309102-129313901 of NCBI accession number NC_000068.7, and the downstream homologous arm sequence (3′ homologous arm, SEQ ID NO: 14, 3899 bp) is identical to nucleotide sequence of 129295411-129299309 of NCBI accession number NC_000068.7. The A fragment comprises a genomic DNA sequence (SEQ ID NO: 12) from a portion of exon 2 (starting from start codon ATG) to a portion of exon 7 (ending at stop codon TAA) of human IL1A gene, which is identical to nucleotide sequence of 112775067-112783770 of NCBI accession number NC_000002.12. The connection between the upstream of the human DNA fragment in the “A fragment” and the mouse sequence is designed as: GGTGTTCTCTTACAGAAATCAAGATGGCCAAAGTTCCAGACATGTTTGAAG (SEQ ID NO: 32), wherein the “G” in sequence “CAAG” is the last nucleotide of the mouse sequence, and the “A” in sequence “ATGG” is the first nucleotide of the human sequence. The connection between the downstream of the human DNA fragment and the mouse sequence is designed as: TACTGGAAAACCAGGCGTAGAAGCAGCCTTATTTCGGGAGTCTATTCACT (SEQ ID NO: 33), wherein the “G” in sequence “GTAG” is the last nucleotide of the human sequence, and the first “A” in sequence “AAGC” is the first nucleotide of the mouse sequence.

The targeting vector also included an antibiotic resistance gene for positive clone screening (neomycin phosphotransferase gene, or Neo), and two Frt recombination sites flanking the antibiotic resistance gene, that formed a Neo cassette. The connection between the upstream of the Neo cassette and the mouse sequence is designed as: GGAGACAGGAGTCGGGGAGACAGAAGGGATGGATATCGAATTCCGAAGTTCCTATT CTCTAGAAAGTATAGGAACTTCAGG (SEQ ID NO: 34), wherein the second “G” in sequence “GATG” is the last nucleotide of the mouse sequence, and the “G” in sequence “GATA” is the first nucleotide of the Neo cassette. The downstream connection of the Neo cassette is designed as: GAAGTTCCTATTCTCTAGAAAGTATAGGAACTTCATCAGTCAGGTACATAATTAGGT GGATCCCACTATGTG\vec{TGAG}AACAAGGCCTTCTAAAATAACTGAGCAAAACCC (SEQ ID NO: 35), wherein the second “G” in sequence “TGTG” is the last nucleotide of the Neo cassette, and the first T in sequence “TGAG” is the first nucleotide of the mouse sequence. In addition, a coding gene with a negative selectable marker (a gene encoding diphtheria toxin A subunit (DTA)) was also inserted downstream of the 3′ homologous arm of the targeting vector. The modified humanized mouse IL1A mRNA sequence is shown as nucleic acids 59-895 of SEQ ID NO: 10, and the expressed protein has the same sequence as human IL1A protein shown in SEQ ID NO: 11.

The targeting vector was constructed, e.g., by restriction enzyme digestion and ligation. The constructed targeting vector sequence was preliminarily verified by restriction enzyme digestion, followed by verification by sequencing. The correct targeting vector was electroporated and transfected into embryonic stem cells of C57BL/6 mice. The positive selectable marker gene was used to screen the cells, and the integration of exogenous genes was confirmed by PCR and Southern Blot.

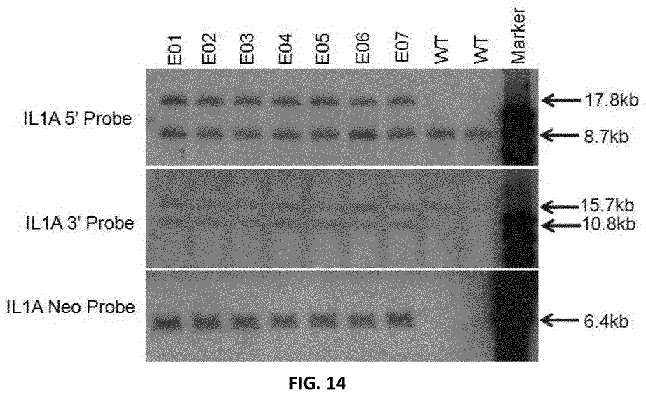

Specifically, PCR primers IL1A ES-F and IL1A ES-R were used for amplification. The positive clones identified by PCR were further confirmed by Southern Blot (digested with NcoI, DraIII, or AseI, respectively, and hybridized with 3 probes) to screen out correct positive clone cells. The length of the probes and the size of target fragments are shown in Table 7, and the results are shown in FIG. 14. All 7 clones, i.e., E01, E02, E03, E04, E05, E06, and E07, were identified as positive clones, which were further verified by sequencing and no random insertions were detected.

TABLE 7
IL1A gene detection probe and target fragment size

| Restriction enzyme | Probe | Wild-type | | Recombinant sequence | |
|---|---|---|---|---|---|
| NcoI | 5′-Probe | 8.7 | kb | 17.8 | kb |
| DraIII | 3′-Probe | 15.7 | kb | 10.8 | kb |

| | | | | |
|---|---|---|---|---|
| AseI | Neo-Probe | — | 6.4 | kb |

The following primers were used in PCR:

ILIAES-F:
(SEQ ID NO: 36)
5′-GCTCGACTAGAGCTTGCGGA-3′
ILIAES-R:
(SEQ ID NO: 37)
5′-GACTTGGACGAGAGAAGGCGTGAG-3′

The following probes were used in Southern Blot assays:

ILIA 5′ Probe:
ILIA 5′ Probe-F:
(SEQ ID NO: 38)
5′-GAAGTAACCCTCCAGAAAAGACTTCCCG-3′
ILIA 5′-Probe-R:
(SEQ ID NO: 39)
5′-GCAACACCAGCTGTGGTCTCTGAT-3′
ILIA 3′-Probe:
ILIA 3′ Probe-F:
(SEQ ID NO: 40)
5′-GGCTTTCCTGATTCTTCTGTACCAAGG-3′
ILIA 3′ Probe-R:
(SEQ ID NO: 41)
5′-GACAGGACCTGACTCTTACTGGTTGTAT-3′

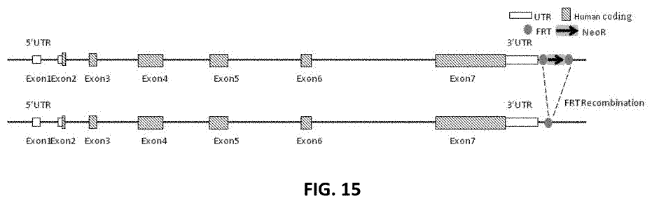

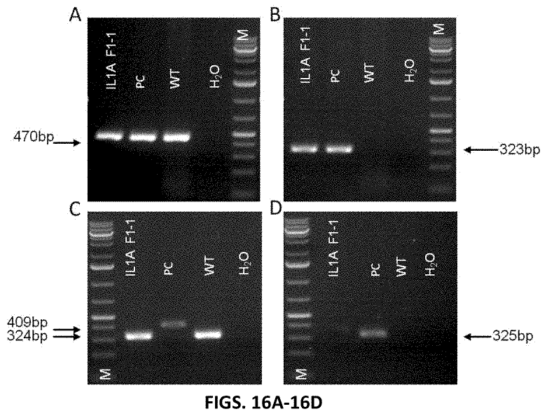

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp mice to remove the positive selectable marker gene (the process diagram is shown in FIG. 15), and then the humanized IL1A homozygous mice expressing human IL1A protein were obtained by breeding with each other. The genotype of the progeny mice can be identified by PCR using primers shown in Table 8. The identification results of exemplary F1 generation mice (Neo cassette-removed) are shown in FIGS. 16A-16D, and the mouse labelled IL1AF1-1 was identified as positive heterozygous clones.

TABLE 8
ILIA gene detection primer sequence

| | | Tm | Fragment |
|---|---|---|---|
| Primer | Sequence (5′-3′) | (° C.) | size (bp) |
| ILIA | TCGAGAGCGCTGTTTCTCATGAAGT | 61 | WT: 470 |
| WT-F | (SEQ ID NO: 42) | | |
| ILIA | GGCTCCACTAGGGTTTGCTCTTCTC | 63 | |
| WT-R | (SEQ ID NO: 43) | | |
| ILIA | TCTTCTTCTGGGAAACTCACGGCAC | 62 | Mut: 323 |
| Mut-F | (SEQ ID NO: 44) | | |
| ILIA | GGCTCCACTAGGGTTTGCTCTTCTC | 63 | |
| WT-R | (SEQ ID NO: 43) | | |
| ILIA | GTTAGGGACAACTGGTCAGCACTCA | 60 | WT: 324 |
| Frt-F | (SEQ ID NO: 45) | | |
| ILIA | TCCATTCAGGAGTCCCATCAGAGCA | 62 | Mut: 409 |
| Frt-R | (SEQ ID NO: 46) | | |
| ILIA | GACAAGCGTTAGTAGGCACATATAC | 57 | Mut: 325 |
| Flp-F2 | (SEQ ID NO: 47) | | |
| ILIA | GCTCCAATTTCCCACAACATTAGT | 58 | |
| Flp-R2 | (SEQ ID NO: 48) | | |

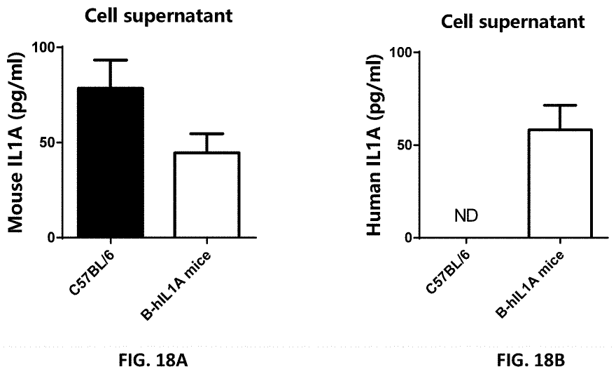

The results indicate that this method can be used to construct humanized IL1A genetically engineered mice that can be passaged stably without random insertion. The expression of human IL1A protein in positive mice can be confirmed, e.g., by enzyme-linked immunosorbent assay (ELISA). For example, ELISA MAX™ Deluxe Set Mouse IL-la Kit and BioLegend LEGEND MAX™ Human IL-la ELISA Kit were used herein. The control group used C57BL/6 wild-type mice, and the experimental group used IL1A humanized heterozygous mice. Specifically, mouse bone marrow samples were collected to isolate monocytes, and 1 μg/mL Lipopolysaccharide (LPS) was used to stimulate the monocytes for 24 hours. Supernatant was collected for ELISA analysis. The test procedure was carried out according to with the instructions of the kits. As shown in FIGS. 18A-18B, expression of mouse IL1A protein was detected in both wild-type mice and IL1A humanized heterozygous mice. However, expression of human IL1A protein was only detected in IL1A humanized heterozygous mice. The results indicate that the humanized IL1A heterozygous mice can successfully express human IL1A protein in vivo.

### Example 3: A Psoriasis Model for Evaluation of In Vivo Drug Efficacy Using Humanized IL1B Mice

Toll-like receptors play an important role in the occurrence and development of psoriasis. Imiquimod is a Toll-like receptor agonist and can be used to model psoriasis. In this example,

C57BL/6 mice and IL1B gene humanized homozygotes mice as described in Example 1 were used to establish an imiquimod-induced psoriasis model. Specifically, C57BL/6 and IL1B humanized mice were randomly divided into 9 groups, each with 8 animals. The grouping is shown in Table 9. The grouping day was set as day DO. On day D-1 (the day before grouping), the hair on the back of the mice was removed by a shaver to expose a 2 cm×4 cm skin area. On days D2-D7, 5% Imiquimod (IMQ) cream (10 mg/cm², smearing area 2 cm×4 cm), was smeared at the back skin area every day for 7 consecutive days. For the treatment groups, dexamethasone (Dexamethasone), Gevokizumab or Canakinumab were randomly selected and administered by subcutaneous (s.c.) injection. The specific grouping and dosing schedule are shown in the table below.

TABLE 9
Grouping and dosing schedule

| | Modeling | | | | Administration | Administration |
|---|---|---|---|---|---|---|
| Group | reagent | Treatment | Dosage | | route | frequency |
| G1 | IMQ | IgG1-kappa | 10 | mg/kg | s.c. | Q3d × 3 |
| (C57BL/6) | | | | | | |
| G2 | IMQ | Gevokizumab | 10 | mg/kg | s.c. | Q3d × 3 |
| (C57BL/6) | | | | | | |
| G3 (hIL1B) | Vaseline | / | / | | / | / |
| G4 (hIL1B) | IMQ | Dexamethasone | 25 | mg/kg | s.c. | QD × 6 |
| G5 (hIL1B) | IMQ | IgG1-kappa | 10 | mg/kg | s.c. | Q3d × 3 |
| G6 (hIL1B) | IMQ | Gevokizumab | 3 | mg/kg | s.c. | Q3d × 3 |
| G7 (hIL1B) | IMQ | Gevokizumab | 10 | mg/kg | s.c. | Q3d × 3 |
| G8 (hIL1B) | IMQ | Canakinumab | 3 | mg/kg | s.c. | Q3d × 3 |
| G9 (hIL1B) | IMQ | Canakinumab | 10 | mg/kg | s.c. | Q3d × 3 |

Starting from day DO, the mice were weighed every day, and photos were taken to record the mouse back skin conditions. The incidence of psoriasis was clinically scored. Scoring items included erythema and scales in mouse skin lesions. Each item was scaled into 0-4 points according to the severity, and the PASI (Psoriasis Area Severity Index) scoring standards were as follows: 0—none; 1—mild; 2—moderate; 3—severe; and 4—extremely severe. A PASI score is a tool used to measure the severity and extent of psoriasis. The average of each score and the average of the total scores of each group of mice were calculated and compared. At the end of the experiment (day D14), the skin specimens of the back and right ear of the mice were sectioned and stained with hematoxylin and eosin (HE). The back erosion, spinous process appearance, hypokeratosis, and mixed inflammatory cell infiltration of each group of mice were scored according to the severity (0.5-2 points): 0.5—slight, 1—slight, 1.5—moderate, and 2—severe. Stromal cell proliferation was also scored (0.5-2 points): 0.5 was 2-4 layers, 1 was 4-6 layers, 1.5 was 6-8 layers, and 2 was 8-10 layers. Appearance of scab: 0.5 points. Result statistics and pathological analysis scores between groups were performed.

The body weight of each group of mice had the same changing trend over time, and they all showed a trend of falling first and then slowly rising. During the experiment, the body weight of each group showed no observable difference. At the end of the experiment, the weight of mice in all groups was close and there was no significant difference. The results of erythema, scaly, and comprehensive PASI scores on the back skin of the mice in each group showed that the pathological development trend of psoriasis in each group of mice was consistent. G2, G4, and G6-G9 groups all exhibited therapeutic effects on psoriasis, and the therapeutic effect of the humanized mouse treatment group (G6-G9) was better than that of the C57BL/6 mouse treatment group (G2), indicating that the treatment of humanized IL1B mice with anti-human IL1B antibody had a better therapeutic effect on psoriasis. The above results prove that the humanized mice as described herein can be used to establish a psoriasis model to evaluate the in vivo efficacy of drugs against human IL1B.

### Example 4: Tumor Models to Evaluate In Vivo Drug Effects Using Humanized IL1B Mice

The tumor models constructed by the humanized mouse prepared herein can be used to test drugs targeting human IL1B. In this example, a monoclonal antibody Canakinumab was selected to verify the efficacy of humanized animal models in vivo. Canakinumab is a first-line drug developed for the treatment of lung cancer. Canakinumab monoclonal antibody is a fully humanized IgG1 monoclonal antibody that specifically binds to human IL1B with high affinity and neutralizes the biological activity of human IL1B by blocking its interaction with IL-1 receptor, thereby preventing IL1B-induced gene activation and production of inflammatory mediators.

In one experiment, the IL1B gene humanized homozygous mice (4-6 week old) prepared in Example 1 were subcutaneously injected with mouse colon cancer cell MC38. After the tumor volume reached about 100 mm³, the mice were randomly divided into a control group and a treatment group (8 mice in each group). The treatment group was administered with Canakinumab (See https://www.cortellis.com/, ID: 320352 for sequence information) at a dose level of 20 mg/kg, and the control group was injected with phosphate-buffered saline (PBS). Canakinumab or PBS were administered by intraperitoneal injection, with a frequency of twice a week (6 times in total). The tumor volume was measured twice a week and body weight of the mice was recorded as well. Euthanasia was performed when tumor volume of a mouse reached 3000 mm³.

Table 10 shows results for this experiment, including the tumor volumes at Day 0 (grouping), Day 14, and Day 21 (the last day of the experiment) after the grouping; the survival rate of the mice; number of tumor-free mice; the Tumor Growth Inhibition value (TGIrv %); and the statistical differences (P value) in mouse body weights and tumor volume between the treatment and control groups.

TABLE 10
Tumor volume, survival status and tumor growth inhibition value

| | | | | | | | P value | |
|---|---|---|---|---|---|---|---|---|
| | Tumor volume (mm³) | | | survival | Tumor- | | Body | Tumor |
| | Day 0 | Day 14 | Day 21 | rate | free | TGI_{TV} % | weight | volume |
| Control | 107 ± 3 | 827 ± 84 | 1640 ± 156 | 8/8 | 0/8 | N/A | N/A | N/A |
| group (G1) | | | | | | | | |
| Treatment | 108 ± 5 | 277 ± 49 | 1116 ± 255 | 8/8 | 0/8 | 34.2% | 0.289 | 0.450 |
| group (G2) | | | | | | | | |

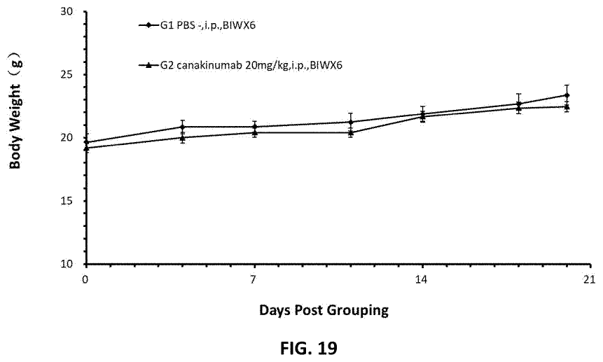

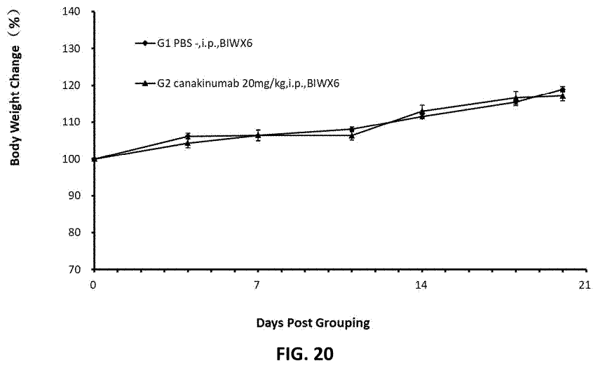

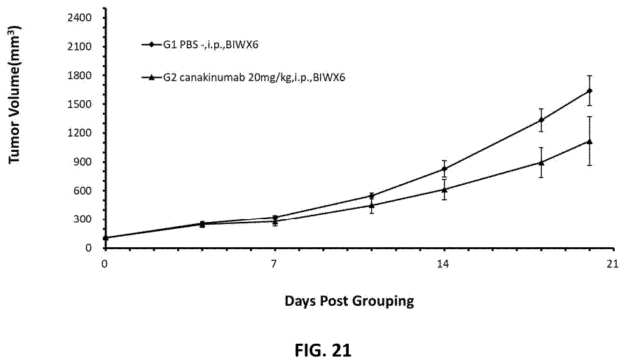

Overall, the mice in each group were grossly healthy. At the end of the experiment (day 21), the body weight of each group increased and there was no significant difference between the groups, indicating that the treatment group mice tolerated the antibody well. There was no significant difference in the average weight gain of mice in the treatment group (G2) and the control group (G1) during the entire experimental period (FIGS. 19-20), indicating that the antibody did not exhibit significant toxic effects on animals. However, with respect to the tumor volume (FIG. 21), in each period, the tumor volume of the treatment group was smaller than that of the control group, and the difference was obvious. Compared with the control group (G1), the treatment group mice showed an inhibitory effect of tumor growth, indicating that Canakinumab had a good inhibitory effect on tumor growth in humanized IL1B animals. It is proved that the humanized IL1B mice prepared by the method described herein can be used for screening anti-human IL1B antibodies and in vivo drug efficacy testing, and used as a living substitute model for in vivo research for the screening, evaluation, and treatment of human IL1B signal pathway regulators.

### Example 5: IL1A/IL1B Double Gene Humanized Mice

The homozygous mice obtained in Example 1 and Example 2 were used for breeding. After multiple generations of screening, IL1A/IL1B double gene humanized mice were obtained. The mice expressed human IL1A protein from humanized homozygous IL1A gene locus, and human IL1B protein from humanized homozygous IL1B gene locus.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric interleukin 1 beta (IL1B).

2. The animal of claim 1, wherein the sequence encoding the human or chimeric IL1B is operably linked to an endogenous regulatory element at the endogenous IL1B gene locus in the at least one chromosome.

3. The animal of claim 1 or 2, wherein the sequence encoding a human or chimeric IL1B is operably linked to an endogenous 5′ untranslated region (5′-UTR).

4. The animal of any one of claims 1-3, wherein the sequence encoding a human or chimeric IL1B comprises a sequence encoding an amino acid sequence that is at least 70%, 75%, 80%, 85%, 90%, 95%, 99%, or 100% identical to human IL1B (SEQ ID NO: 4).

5. The animal of any one of claims 1-4, wherein the sequence comprises a sequence that is at least 70%, 75%, 80%, 85%, 90%, 95%, 99%, or 100% identical to SEQ ID NO: 15, 16, 17, or 18.

6. The animal of any one of claims 1-5, wherein the animal is a mammal, e.g., a monkey, a rodent, or a mouse.

7. The animal of claim 6, wherein the mammal is a mouse.

8. The animal of any one of claims 1-7, wherein the animal does not express endogenous IL1B.

9. The animal of any one of claims 1-8, wherein the animal has one or more cells expressing human or chimeric IL1B.

10. The animal of any one of claims 1-9, wherein the expressed human or chimeric IL1B can bind to human IL-1 receptor type I (IL1R1).

11. The animal of any one of claims 1-9, wherein the expressed human or chimeric IL1B can bind to endogenous IL1R1.

12. A genetically-modified, non-human animal, wherein the genome of the animal comprises a replacement of a sequence encoding a region of endogenous IL1B with a sequence encoding a corresponding region of human IL1B at an endogenous IL1B gene locus.

13. The animal of claim 12, wherein the sequence encoding the corresponding region of human IL1B is operably linked to an endogenous regulatory element at the endogenous IL1B locus.

14. The animal of claim 12 or 13, wherein the animal does not express endogenous IL1B, and the animal has one or more cells expressing human or chimeric IL1B.

15. The animal of any one of claims 12-14, wherein the replaced sequence encoding a region of endogenous IL1B comprises exon 1, exon 2, exon 3, exon 4, exon 5, exon 6, and/or exon 7, or a part thereof, of endogenous IL1B gene.

16. The animal of claim 15, wherein the animal is a mouse, and the replaced sequence starts within exon 2 and ends within exon 7 of endogenous mouse IL1B gene.

17. The animal of any one of claims 12-16, wherein the animal is heterozygous with respect to the replacement at the endogenous IL1B gene locus.

18. The animal of any one of claims 12-16, wherein the animal is homozygous with respect to the replacement at the endogenous IL1B gene locus.

19. A method for making a genetically-modified, non-human animal, comprising:
replacing in at least one cell of the animal, at an endogenous IL1B gene locus, a sequence encoding a region of an endogenous IL1B with a sequence encoding a corresponding region of human IL1B.

20. The method of claim 19, wherein the sequence encoding the corresponding region of human IL1B comprises exon 2, exon 3, exon 4, exon 5, exon 6, and/or exon 7, or a part thereof, of a human IL1B gene.

21. The method of claim 19 or 20, wherein the sequence encoding the corresponding region of human IL1B encodes an amino acid sequence that is at least 70%, 75%, 80%, 85%, 90%, 95%, 99%, or 100% identical to SEQ ID NO: 4.

22. The method of any one of claims 19-21, wherein the endogenous IL1B locus comprises exon 1, exon 2, exon 3, exon 4, exon 5, exon 6, and/or exon 7, or a part thereof, of the endogenous IL1B gene.

23. A non-human animal comprising at least one cell comprising a nucleotide sequence encoding an exogenous IL1B polypeptide, wherein the exogenous IL1B polypeptide comprises at least 50 contiguous amino acid residues that are identical to the corresponding contiguous amino acid sequence of a human IL1B, wherein the animal expresses the exogenous IL1B.

24. The animal of claim 23, wherein the exogenous IL1B polypeptide comprises an amino acid sequence that is at least 90%, 95%, or 99% identical to SEQ ID NO: 4.

25. The animal of claim 23 or 24, wherein the nucleotide sequence is operably linked to an endogenous IL1B regulatory element of the animal.

26. The animal of any one claims 23-25, wherein the nucleotide sequence is integrated to an endogenous IL1B gene locus of the animal.

27. The animal of any one of claims 23-26, wherein the animal in its genome comprises, preferably from 5′ to 3′: a mouse 5′ UTR, a sequence encoding the exogenous IL1B polypeptide, and a mouse 3′ UTR.

28. A method of making a genetically-modified non-human animal cell that expresses a chimeric IL1B, the method comprising:
replacing at an endogenous IL1B gene locus, a nucleotide sequence encoding a region of endogneous IL1B with a nucleotide sequence encoding a corresponding region of human IL1B, thereby generating a genetically-modified non-human animal cell that includes a nucleotide sequence that encodes the chimeric IL1B, wherein the non-human animal cell expresses the chimeric IL1B.

29. The method of claim 28, wherein the nucleotide sequence encoding the chimeric IL1B is operably linked to an endogenous IL1B regulatory region, e.g., promoter.

30. The animal of any one of claims 1-18 and 23-27, wherein the animal further comprises a sequence encoding an additional human or chimeric protein.

31. The animal of claim 30, wherein the additional human or chimeric protein is interleukin 1 alpha (IL1A), IL-1 receptor type I (IL1R1), interleukin-1 receptor accessory protein (IL1RAP), programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

32. The animal of claim 30, wherein the additional human or chimeric protein is IL1A and the animal expresses the human or chimeric IL1A.

33. The method of any one of claims 19-22, 28, and 29, wherein the animal or mouse further comprises a sequence encoding an additional human or chimeric protein.

34. The method of claim 33, wherein the additional human or chimeric protein is IL1A, IL1R1, IL1RAP, PD-1, CTLA-4, LAG-3, IL15 receptor, BTLA, PD-L1, CD3, CD27, CD28, CD47, CD137, CD154, TIGIT, TIM-3, GITR, SIRPα or OX40.

35. The method of claim 33, wherein the additional human or chimeric protein is IL1A and the animal expresses the human or chimeric IL1A.

36. A method of determining effectiveness of an anti-IL1B antibody for treating an allergic disorder, comprising:
a) administering the anti-IL1B antibody to the animal of any one of claims 1-18, 23-27, and 30-32, wherein the animal has the allergic disorder; and
b) determining effects of the anti-IL1B antibody in treating the allergic disorder.

37. The method of claim 36, wherein the allergic disorder is allergy, asthma, and/or atopic dermatitis.

38. A method of determining effectiveness of an anti-IL1B antibody for reducing an inflammation, comprising:
a) administering the anti-IL1B antibody to the animal of any one of claims 1-18, 23-27, and 30-32, wherein the animal has the inflammation; and
b) determining effects of the anti-IL1B antibody for reducing the inflammation.

39. A method of determining effectiveness of an agent for treating an autoimmune disorder, comprising:
a) administering the agent to the animal of any one of claims 1-18, 23-27, and 30-32, wherein the animal has the autoimmune disorder; and
b) determining effects of the agent for treating the autoimmune disorder.

40. The method of claim 39, wherein the autoimmune disorder is rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

41. The method of claim 39, wherein the autoimmune disorder is psoriasis.

42. The method of claim 41, wherein the animal is a mouse and the psoriasis is induced by treating the mouse with imiquimod (IMQ).

43. The method of claim 41 or 42, wherein the agent is a corticosteroid (e.g., dexamethasone).

44. The method of claim 41 or 42, wherein the agent is an anti-IL1B antibody.

45. The method of claim 44, wherein the anti-IL1B antibody is Gevokizumab or Canakinumab.

46. The method of any one of claims 41-45, wherein the effects are evaluated by clinical scores (e.g., Psoriasis Area Severity Index) and/or hematoxylin and eosin (HE) staining.

47. A method of determining effectiveness of an agent for treating an autoinflammatory disease, comprising:
a) administering the agent to the animal of any one of claims 1-18, 23-27, and 30-32, wherein the animal has the autoinflammatory disease; and
b) determining effects of the agent for treating the autoinflammatory disease.

48. The method of claim 47, wherein the autoinflammatory disease is tumor necrosis factor receptor associated periodic syndrome (TRAPS), hyperimmunoglobulin D syndrome (HIDS)/mevalonate kinase deficiency (MKD), familial mediterranean fever (FMF), Still's disease, adult-onset Still's disease (AOSD), autoinflammatory periodic fever syndromes, cryopyrin-associated periodic syndromes (CAPS), Familial Cold Autoinflammatory Syndrome (FCAS), Muckle-Wells syndrome (MWS), Neonatal-Onset Multisystem Inflammatory Disease (NOMID), Deficiency of the interleukin-1 receptor antagonist (DIRA), or gouty arthritis.

49. The method of claim 47 or 48, wherein the agent is an anti-IL1B antibody.

50. A method of determining effectiveness of an anti-IL1B antibody for treating a cancer, comprising:
a) administering the anti-IL1B antibody to the animal of any one of claims 1-18, 23-27, and 30-32, wherein the animal has the cancer; and
b) determining inhibitory effects of the anti-IL1B antibody for treating the cancer.

51. The method of claim 50, wherein the cancer is a tumor, and determining the inhibitory effects of the treatment involves measuring the tumor volume in the animal.

52. The method of claim 50 or 51, wherein the cancer comprises one or more cancer cells that are injected into the animal.

53. The method of any one of claims 50-52, wherein the cancer is breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, melanoma, or bone cancer.

54. The method of claim 53, wherein the cancer is colorectal cancer, lung cancer, or melanoma.

55. A method of determining toxicity of an anti-IL1B antibody, the method comprising
a) administering the anti-IL1B antibody to the animal of any one of claims 1-18, 23-27, and 30-32; and
b) determining weight change of the animal.

56. The method of claim 55, the method further comprising performing a blood test (e.g., determining red blood cell count).

57. A genetically-modified, non-human animal whose genome comprises at least one chromosome comprising a sequence encoding a human or chimeric interleukin 1 alpha (IL1A).

58. The animal of claim 57, wherein the sequence encoding the human or chimeric IL1A is operably linked to an endogenous regulatory element at the endogenous IL1A gene locus in the at least one chromosome.

59. The animal of claim 57 or 58, wherein the sequence encoding a human or chimeric IL1A is operably linked to an endogenous 5′ untranslated region (5′-UTR).

60. The animal of any one of claims 57-59, wherein the sequence encoding a human or chimeric IL1A comprises a sequence encoding an amino acid sequence that is at least 70%, 75%, 80%, 85%, 90%, 95%, 99%, or 100% identical to human IL1A (SEQ ID NO: 11).

61. The animal of any one of claims 57-60, wherein the sequence comprises a sequence that is at least 70%, 75%, 80%, 85%, 90%, 95%, 99%, or 100% identical to SEQ ID NO: 32, 33, 34, or 35.

62. The animal of any one of claims 57-61, wherein the animal is a mammal, e.g., a monkey, a rodent, or a mouse.

63. The animal of claim 62, wherein the mammal is a mouse.

64. The animal of any one of claims 57-63, wherein the animal does not express endogenous IL1A.

65. The animal of any one of claims 57-64, wherein the animal has one or more cells expressing human or chimeric IL1A.

66. The animal of any one of claims 57-65, wherein the expressed human or chimeric IL1A can bind to human IL-1 receptor type I (IL1R1).

67. The animal of any one of claims 57-65, wherein the expressed human or chimeric IL1A can bind to endogenous IL1R1.

68. A genetically-modified, non-human animal, wherein the genome of the animal comprises a replacement of a sequence encoding a region of endogenous IL1A with a sequence encoding a corresponding region of human IL1A at an endogenous IL1A gene locus.

69. The animal of claim 68, wherein the sequence encoding the corresponding region of human IL1A is operably linked to an endogenous regulatory element at the endogenous IL1A locus.

70. The animal of claim 68 or 69, wherein the animal does not express endogenous IL1A, and the animal has one or more cells expressing human or chimeric IL1A.

71. The animal of any one of claims 68-70, wherein the replaced sequence encoding a region of endogenous IL1A comprises exon 1, exon 2, exon 3, exon 4, exon 5, exon 6, and/or exon 7, or a part thereof, of endogenous IL1A gene.

72. The animal of claim 71, wherein the animal is a mouse, and the replaced sequence starts within exon 2 and ends within exon 7 of endogenous mouse IL1A gene.

73. The animal of any one of claims 68-72, wherein the animal is heterozygous with respect to the replacement at the endogenous IL1A gene locus.

74. The animal of any one of claims 68-72, wherein the animal is homozygous with respect to the replacement at the endogenous IL1A gene locus.

75. A method for making a genetically-modified, non-human animal, comprising:
replacing in at least one cell of the animal, at an endogenous IL1A gene locus, a sequence encoding a region of an endogenous IL1A with a sequence encoding a corresponding region of human IL1A.

76. The method of claim 75, wherein the sequence encoding the corresponding region of human IL1A comprises exon 2, exon 3, exon 4, exon 5, exon 6, and/or exon 7, or a part thereof, of a human IL1A gene.

77. The method of claim 75 or 76, wherein the sequence encoding the corresponding region of human IL1A encodes an amino acid sequence that is at least 70%, 75%, 80%, 85%, 90%, 95%, 99%, or 100% identical to SEQ ID NO: 11.

78. The method of any one of claims 75-77, wherein the endogenous IL1A locus comprises exon 1, exon 2, exon 3, exon 4, exon 5, exon 6, and/or exon 7, or a part thereof, of the endogenous IL1A gene.

79. A non-human animal comprising at least one cell comprising a nucleotide sequence encoding an exogenous IL1A polypeptide, wherein the exogenous IL1A polypeptide comprises at least 50 contiguous amino acid residues that are identical to the corresponding contiguous amino acid sequence of a human IL1A, wherein the animal expresses the exogenous IL1A.

80. The animal of claim 79, wherein the exogenous IL1A polypeptide comprises an amino acid sequence that is at least 90%, 95%, or 99% identical to SEQ ID NO: 11.

81. The animal of claim 79 or 80, wherein the nucleotide sequence is operably linked to an endogenous IL1A regulatory element of the animal.

82. The animal of any one claims 79-81, wherein the nucleotide sequence is integrated to an endogenous IL1A gene locus of the animal.

83. The animal of any one of claims 79-82, wherein the animal in its genome comprises, preferably from 5′ to 3′: a mouse 5′ UTR, a sequence encoding the exogenous IL1A polypeptide, and a mouse 3′ UTR.

84. A method of making a genetically-modified non-human animal cell that expresses a chimeric IL1A, the method comprising:
replacing at an endogenous IL1A gene locus, a nucleotide sequence encoding a region of endogenous IL1A with a nucleotide sequence encoding a corresponding region of human IL1A, thereby generating a genetically-modified non-human animal cell that includes a nucleotide sequence that encodes the chimeric IL1A, wherein the non-human animal cell expresses the chimeric IL1A.

85. The method of claim 84, wherein the nucleotide sequence encoding the chimeric IL1A is operably linked to an endogenous IL1A regulatory region, e.g., promoter.

86. The animal of any one of claims 57-74 and 79-83, wherein the animal further comprises a sequence encoding an additional human or chimeric protein.

87. The animal of claim 86, wherein the additional human or chimeric protein is interleukin 1 beta (IL1B), IL-1 receptor type I (IL1R1), interleukin-1 receptor accessory protein (IL1RAP), programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

88. The animal of claim 86, wherein the additional human or chimeric protein is IL1B and the animal expresses the human or chimeric IL1B.

89. The method of any one of claims 75-78, 84, and 85, wherein the animal or mouse further comprises a sequence encoding an additional human or chimeric protein.

90. The method of claim 89, wherein the additional human or chimeric protein is IL1B, IL1R1, IL1RAP, PD-1, CTLA-4, LAG-3, IL15 receptor, BTLA, PD-L1, CD3, CD27, CD28, CD47, CD137, CD154, TIGIT, TIM-3, GITR, SIRPα or OX40.

91. The method of claim 89, wherein the additional human or chimeric protein is IL1B and the and the animal expresses the human or chimeric IL1B.

92. A method of determining effectiveness of an anti-IL1A antibody for treating an allergic disorder, comprising:
a) administering the anti-IL1A antibody to the animal of any one of claims 57-74, 79-83, and 86-88, wherein the animal has the allergic disorder; and
b) determining effects of the anti-IL1A antibody in treating the allergic disorder.

93. The method of claim 92, wherein the allergic disorder is allergy, asthma, and/or atopic dermatitis.

94. A method of determining effectiveness of an anti-IL1A antibody for reducing an inflammation, comprising:
a) administering the anti-IL1A antibody to the animal of any one of claims 57-74, 79-83, and 86-88, wherein the animal has the inflammation; and
b) determining effects of the anti-IL1A antibody for reducing the inflammation.

95. A method of determining effectiveness of an agent for treating an autoimmune disorder, comprising:
a) administering the agent to the animal of any one of claims 57-74, 79-83, and 86-88, wherein the animal has the autoimmune disorder; and
b) determining effects of the agent for treating the autoimmune disorder.

96. The method of claim 95, wherein the autoimmune disorder is rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

97. The method of claim 95, wherein the autoimmune disorder is psoriasis.

98. The method of any one of claims 95-97, wherein the agent is a corticosteroid (e.g., dexamethasone) or an anti-IL1A antibody.

99. A method of determining effectiveness of an agent for treating an autoinflammatory disease, comprising:
a) administering the agent to the animal of any one of claims 57-74, 79-83, and 86-88, wherein the animal has the autoinflammatory disease; and
b) determining effects of the agent for treating the autoinflammatory disease.

100. The method of claim 99, wherein the autoinflammatory disease is tumor necrosis factor receptor associated periodic syndrome (TRAPS), hyperimmunoglobulin D syndrome (HIDS)/mevalonate kinase deficiency (MKD), familial mediterranean fever (FMF), Still's disease, adult-onset Still's disease (AOSD), autoinflammatory periodic fever syndromes, cryopyrin-associated periodic syndromes (CAPS), Familial Cold Autoinflammatory Syndrome (FCAS), Muckle-Wells syndrome (MWS), Neonatal-Onset Multisystem Inflammatory Disease (NOMID), Deficiency of the interleukin-1 receptor antagonist (DIRA), or gouty arthritis.

101. The method of claim 99 or 100, wherein the agent is an anti-IL1A antibody or anti-IL1B antibody.

102. A method of determining effectiveness of an anti-IL1A antibody for treating a cancer, comprising:
a) administering the anti-IL1A antibody to the animal of any one of claims 57-74, 79-83, and 86-88, wherein the animal has the cancer; and
b) determining inhibitory effects of the anti-IL1A antibody for treating the cancer.

103. The method of claim 102, wherein the cancer is a tumor, and determining the inhibitory effects of the treatment involves measuring the tumor volume in the animal.

104. The method of claim 102 or 103, wherein the cancer comprises one or more cancer cells that are injected into the animal.

105. The method of any one of claims 102-104, wherein the cancer is a solid tumor, breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, melanoma, refractory cancer, or bone cancer.

106. A method of determining toxicity of an anti-IL1A antibody, the method comprising
a) administering the anti-IL1A antibody to the animal of any one of claims 57-74, 79-83, and 86-88; and
b) determining weight change of the animal.

107. The method of claim 106, the method further comprising performing a blood test (e.g., determining red blood cell count).

108. A protein comprising an amino acid sequence, wherein the amino acid sequence is one of the following:
(a) an amino acid sequence set forth in SEQ ID NO: 2, 4, 9, or 11;
(b) an amino acid sequence that is at least 90% identical to SEQ ID NO: 2, 4, 9, or 11;
(c) an amino acid sequence that is at least 91%, 92%, 93%, 94%, 95%, 96%, 97%, 98%, or 99% identical to SEQ ID NO: 2, 4, 9, or 11;
(d) an amino acid sequence that is different from the amino acid sequence set forth in SEQ ID NO: 2, 4, 9, or 11 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2, 4, 9, or 11.

109. A nucleic acid comprising a nucleotide sequence, wherein the nucleotide sequence is one of the following:
(a) a sequence that encodes the protein of claim 108;
(b) SEQ ID NO: 1, 3, 5, 6, 7, 8, 10, 12, 13, or 14;
(c) a sequence that is at least 90% identical to SEQ ID NO: 1, 3, 5, 6, 7, 8, 10, 12, 13, or 14; and
(d) a sequence that is at least 91%, 92%, 93%, 94%, 95%, 96%, 97%, 98%, or 99% identical to 1, 3, 5, 6, 7, 8, 10, 12, 13, or 14.

110. A cell comprising the protein of claim 108 and/or the nucleic acid of claim 109.

111. An animal comprising the protein of claim 108 and/or the nucleic acid of claim 109.


SEQUENCE LISTING (50 sequences)

SEQ ID NO: 1 - DNA, 1348 bp, Mus musculus
aacaaaccctgcagtggttcgaggcctaataggctcatctgggatcctctccagccaagcttccttgtgcaagtgtctgaagcagctatggcaactgttcctgaactcaactgtgaaatgccaccttttgacagtgatgagaatgacctgttctttgaagttgacggaccccaaaagatgaagggctgcttccaaacctttgacctgggctgtcctgatgagagcatccagcttcaaatctcgcagcagcacatcaacaagagcttcaggcaggcagtatcactcattgtggctgtggagaagctgtggcagctacctgtgtctttcccgtggaccttccaggatgaggacatgagcaccttcttttccttcatctttgaagaagagcccatcctctgtgactcatgggatgatgatgataacctgctggtgtgtgacgttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaaagtatgggctggactgtttctaatgccttccccagggcatgttaaggagctcccttttcgtgaatgagcagacagctcaatctccaggggactccttagtcctcggccaagacaggtcgctcagggtcacaagaaaccatggcacattctgttcaaagagagcctgtgttttcctccttgcctctgatgggcaaccacttacctatttatttatgtatttattgattggttgatctatttaagttgattcaaggggacattaggcagcactctctagaacagaacctagctgtcaacgtgtgggggatgaattggtcatagcccgcactgaggtctttcattgaagctgagaataaataggttcctataatatggatgagactttttatgaatgaagcaccagcacattgctttgatgagtatgaaataaatttcattaaaacaaacaaac

SEQ ID NO: 2 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 3 - DNA, 1507 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaagaaaaaaaaa

SEQ ID NO: 4 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 5 - DNA, 5869 bp, Artificial Sequence
  nucleotide sequence of 112830361-112836229 of NCBI accession number NC_000002.12
atggcagaagtacctgagctcgccagtgaaatgatggcttattacaggtcagtggagacgctgagaccagtaacatgagcaggtctcctctttcaagagtagagtgttatctgtgcttggagaccagatttttcccctaaattgcctctttcagtggcaaacagggtgccaagtaaatctgatttaaagactactttcccattacaagtccctccagccttgggacctggaggctatccagatgtgttgttgcaagggcttcctgcagaggcaaatggggagaaaagactccaagcccacaatacaaggaatccctttgcaaagtgtggcttggagggagagggagagctcagattttagctgactctgctgggctagaggttaggcctcaagatccaacagggagcacccagggtgcccacctgccaggcctagaatctgccttctggactgttctgcgcatatcactgtgaaacttgccaggtgtttcaggcagctttgagaggcaggctgtttgcagtttcttatgaacagtcaagtcttgtacacagggaaggaaaaataaacctgtttagaagacataattgagacatgtccctgtttttattacagtggcaatgaggatgacttgttctttgaagctgatggccctaaacagatgaaggtaagactatgggtttaactcccaacccaaggaagggctctaacacagggaaagctcaaagaagggagttctgggccactttgatgccatggtattttgttttagaaagactttaacctcttccagtgagacacaggctgcaccacttgctgacctggccacttggtcatcatatcaccacagtcactcactaacgttggtggtggtggccacacttggtggtgacaggggaggagtagtgataatgtttcccatttcatagtaggaagacaaccaagtcttcaacataaatttgattatccttttaagagatggattcagcctatgccaatcacttgagttaaactctgaaaccaagagatgatcttgagaactaacatatgtctaccccttttgagtagaatagttttttgctacctggggtgaagcttataacaacaagacatagatgatataaacaaaaagatgaattgagacttgaaagaaaaccattcacttgctgtttgaccttgacaagtcattttacccgctttggacctcatctgaaaaataaagggctgagctggatgatctctgagattccagcatcctgcaacctccagttctgaaatattttcagttgtagctaagggcatttgggcagcaaatggtcatttttcagactcatccttacaaagagccatgttatattcctgctgtcccttctgttttatatgatgctcagtagccttcctaggtggcccagccatcagcctagctaggtcagttgtgcaggttgggaggcagccacttttctctggctttattttattccagtttgtgatagcctcccctagcctcataatccagtcctcaatcttgttaaaaacatatttctttagaagttttaagactggcataacttgttggctgcagctgtgggaggagcccattggcttgtctgcctggcctttgcccccattgcctcttccagcagcttggctctgctccaggcaggaaattctctcctgctcaactttcttttgtgcacttacaggtctctttaactgtctttcaagcctttgaaccattatcatgccttaaggcaacctcagtgaagccttaatacggagcttctctgaataagaggaaagtggtaacatttcacaaaaagtactctcacaggatttgcagaatgcctatgagacagtgttatgaaaaaggaaaaaaaagaacagtgtagaaaaattgaatacttgctgagtgagcataggtgaatggaaaatgttatggtcatctgcatgaaaaagcaaatcatagtgtgacagcattagggatacaaaaagatatagagaaggtatacatgtatggtgtaggtggggcatgtacaaaaaagatgaacaaagtagaaatgggatttattctaaaagaatagcctgtaaggtgtcagaaagcccacattctagtcttgagtctgcctctaacctgctgtgtgcccttgagtacacacttaacctccttgagcttcagagagggataatctttttattttattttattttattttgttttgttttgttttgttttgttttatgagacagagtctcactctgttgcccaggctggagtgcagtggtacaatcttggcttactgcatcctccacctcctgagttcaagcgattctccttcctcagtctcctgaatagctaggattacaggtgcaccccaccacacccagctaatttttgtatttttagtagagaaggggtttcgccatgttggccaggctggttttgaagtcctgacctaaatgattcatccacctcggcttcccaaagtgctgggattacaggcatgagccaccacgcctggcccagagagggatgatctttagaagctcgggattctttcaagccctttcctcctctctgagctttctactctctgatgtcaaagcatggttcctggcaggaccacctcaccaggctccctccctcgctctctccgcagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaaggtagttagccaagagcaggcagtagatctccacttgtgtcctcttggaagtcatcaagccccagccaactcaattcccccagagccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccgcctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccacccaagtctcttgggcatagtgcaatgtcaattcttccacaatatggggtcatttgatggacatggcctaactgcctgtgggttctctcttcctgttgttgaggctgaaacaagagtgctggagcgataatgtgtccatccccctccccagtcttccccccttgccccaacatccgtcccacccaatgccaggtggttccttgtagggaaattttaccgcccagcaggaacttatatctctccgctgtaacgggcaaaagtttcaagtgcggtgaacccatcattagctgtggtgatctgcctggcatcgtgccacagtagccaaagcctctgcacaggagtgtgggcaactaaggctgctgactttgaaggacagcctcactcagggggaagctatttgctctcagccaggccaagaaaatcctgtttctttggaatcgggtagtaagagtgatcccagggcctccaattgacactgctgtgactgaggaagatcaaaatgagtgtctctctttggagccactttcccagctcagcctctcctctcccagtttcttcccatgggctactctctgttcctgaaacagttctggtgcctgatttctggcagaagtacagcttcacctctttcctttccttccacattgatcaagttgttccgctcctgtggatgggcacattgccagccagtgacacaatggcttccttccttccttccttcagcatttaaaatgtagaccctctttcattctccgttcctactgctatgaggctctgagaaaccctcaggcctttgaggggaaaccctaaatcaacaaaatgaccctgctattgtctgtgagaagtcaagttatcctgtgtcttaggccaaggaacctcactgtgggttcccacagaggctaccaaattacatgtatcctactcatggggcctaggggttggggtgaccctgcactgctgtgtccctaaccacaagacccccttctttcttcagtggtgttctccatgtcctttgtacaaggagaagaaagtaatgacaaaatacctgtggccttgggcctcaaggaaaagaatctgtacctgtcctgcgtgttgaaagatgataagcccactctacagctggaggtaagtgaatgctatggaatgaagcccttctcagcctcctgctaccacttattcccagacaaccaccttctccccgcccccatccctaggaaaagctgggaacaggtctatttgacaattttgcattaatgtaaataaatttaacataatttttaactgcgtgcaaccttcaatcctgctgcagaaaattaaatcattttgccgatgttattatgtcctaccatagttacaaccccaacagattatatattgttagggctgctctcatttgatagacaccttgggaaatagatgacttaaagggtcccattatcatgtccactccactcccaaaattaccaccactatcacctccagctttctcagcaaaagcttcatttccaagttgatgtcattctaggaccataaggaaaaatacaataaaaagcccctggaaactaggtacttcaagaagctctagcttaattttcacccccccaaaaaaaaaaaattctcacctacattatgctcctcagcatttggcactaagttttagaaaagaagaagggctcttttaataatcacacggaaagttgggggcccagttacaactcaggagtctggctcctgatcatgtgacctgctcgtcagtttcctttctggccaacccaaagaacatctttcccatagcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 6 - DNA, 4941 bp, Artificial Sequence
  5′ homologous arm
gtgcatgttccaacgtcaaagatgcactcaagatcttctgggatcctctaaggggcttgagttacttctgcagctcagccctttgtagcacaaacagcttgtcttttaggatttggctggctccactccactgctgctgctgttcttgatggtcatcccatggtacttgcatctccaaaatgctggggtcttctgctgcaactgggctggacttataccagtactctcctgggctctcttcatggtgacaagcctcaacttctctgtatgaccctttcaatcctgggccttcagctgccactgaggctgtattgtcagtgccaagactcagctgctcttccatgaaccagtgtcacctgggtggctcttccacaggaccaaatttggctgccagtggagaaatacaactttggccatctctggaaaacagcttctgtgtgctctcagaaaacacttcccagaagatttcacctcaataatgctggacttttcttagtcactgctaatttctcagctccagctcaccagcactgagtatctaagcaaagcaaaggtttcatttttagtggttctggaatcttgtttatcgatgcctattcttcagccccagctaatgagatattatgttatcacggaatcttaattcaatataacaaatggccctgaagaagtctttaagcttccttctgaagcttcacaagtcaggcctccatctttgtgttgccctcaacgtccctatcttccaagttcctaggaacagctcaccaagaattgaccactctatgggttttcttgtacaaagtccttccaaaacaatatgctcaggtctgtcacagtcatgtcacagtaaatcttggtgccaattcattttcatttaggttactattactataatgaaactccatgatcaaagcaacttggggaggaaaggatgtattctgcttacatttccacatcacagtttatcatcaaaggaagtcaggacaggaactcaagcaaggcaggaacctggaaccaggagctgatgcagcgatcatggagggatgctgctcactggcttgctcctcatggcttgtcaacctactttcttatagaacccaggaccaccagcccagggatggcaccacccacaacaggctgatttctccccaagtaaataccaattaataaaatttcctacaggcttgcctacagacagatcttttggatgtattttgtcagttaaggctcccttctctctgatgattctagcttgtgtctagttgacataaaactaaccaggacagaaaagatgagagggaaagaacagacccctaaggcctgtgctaagtcgtcaacttaaggaataagacaaggtctggagaaagtaatgaggacagtcattgcttagctctgttctgagcaagaggataagtaaagaagatgtagaacacatacatcaactgggcctgggagctcgtgcctgtaatctcagtccttgggagacaaatgcaggagaattgtcatgtacttgaagccagtctgggctgcacagtagtcatggttatcacagcaatagaaagtaagtaaaacaggtagcaaggcactttcagctttgaagaaatgcctgcctccatcttggaggaatgagatgtcagaacagagggaaccttacagcttaaaagtgctgagtgagtcaagagctgaaaagttccccaaaagctagagtgcccgtcaccatcctggctttgccgacttcctcttttgctttgttcatttcctttgccaacatcatcatcagtatcgtcatcactatgccacacccccagcataacaatttctatagtgagttatttcttctactcattggggaccaaaaaggaagtgtggtctgagagacagggtttgatatacatgttgtgcaacttgcctgctctataacgacaaggggaggaaatttggagcccaagtcacagggccaggatgaggtttgatagaacaatagagtaccagaggcattgcccagtagttccaaaatctccctctagaagcaaaagaatcatcaaccagatcattgcctcctcccagacaaacctcctcccatctcttcatctcttactcacgattaaatggccattcgtcttcatgcatgtgccttcctccaaatcctcccagacaaccactcctctcaggcatcagctcaagggtttaggagtgttataactagcagatggtgaagaagattctgtaactagactgagctcaaggctcctgaaaaatcatccagggagaagggacttggagctgacactcaggcccctagttacccttctctgtcccctggttttcaccatccctggttcaactcacatcagcagaccaaaggagtctccaataatctctgtcaggacagcccataaaatcatcagaccttccagccctgcacacaggctctgaggaaggtgttagtccctccaggcatagtttgaaatgtggaccctgtgaaggcagaacagaaaaatgaaccagatggcccagacaggacctctggattgtctgcagaactagataatacatcttataagaccttagtgctgagaactgaccattgctcaccaaactcaaaagcaactgagaccctgaaccatcttcagatttcaaatcaaacatatagctggtcaaaggcaggattcttctgtttgccttcctgaaatcgagatgctgtgaaccaaattaggcaaaagaaggctgcctagtaagtaaaccttgtttcatagtagagccttgtctattcctccttccaattctgtctgtctatttcccttcagtgctgcagaataagctcagtaaccaaaacatactaggtacaaactcatctgaatgaacacattgccaaattcctactcacccgggctagctcctactgcctgcatccatctgccaggcactgtggagacctggctctaatggagtccacagactctctgagggctcagcaaaggagttaggtttccactgaggattctactatactgtagatgtgcccaagagactgtatgcagcattcatatggcctggttgctcattccatccaagcaagaagagctcccctgggtaggctccctgggctctctgagttagcagtctagtgatgcttgatatggccaagagacttggtctccccagatcttatagaaacaagaattttccaaaacaattttttaggcaaagactatctcttcactttttaagatggactgtgctcatgaacaggcagatgcctcgttcaccacctttgcactgtgcaacttaattcaggctcattctgctgatcacctagcactgatatggtttcaacatgagactggctatggtattataagtaccctggcagggcaggaaagcaggagtgggtgggtgagtgggggagcatcctcatagaggcaggggagggggagaggatagggggtttccggaggggagacctggaaaagggataacatttgaaatgtaaataaagaaaatatccaataaaagaaaaaaataagcaccctggcattatcagactgcataggcttgcttccagagttccctgaccctatgataagtctacactgatacctgcatactgtgtgtgccctgacccacacaaggaagtgcgtgtctctccagaagcccctgctaacacagttgatggagagcacagaagcaccatccagttaccaaactccaactgcaaagctccctcagcttaagcacaaggaggcgagagaggtgacacacttctgggtgtgcatctacgtgcctacctttgttccgcacatcctgacttaaaatgtacagctaacccaggaaaacccaatatttttaatattgacaccatctgcacaattgtccagggggaaataatgcccatttccaccacgatgacacacttgcgaatgtgtcactatctgccaccccttgacttccagggattagaaattatttcagggtagcaatagcctcttcccctaagaattcccatcaagcttctcccccctcccccacccttcagttttgttgtgaaatcagttaacccaagggaaaatttcacagctcttcacttctgctttttaggactataaaacaagggagggaaaacaagttggacaacaaaccctgcagtggttcgaggcctaataggctcatctgggatcctctccagccaagcttccttgtgcaagtaagtctctctctctctctgtctctctctctctctgtctctctctatctctctctctctgtctctctctctctgtctctgtctgtctctctctctgtctctgtctgtctatctctctctctctctgtctgtctctgtctctctctgtctgtctctctctgtctctctgtctctctctctgtctctctctgtctctctctctctctctctttcccccccccccactgatggactttgggctttttatttataaaaagtagctgtacctccatggtttgtgaagtactctggaaatgagtactgtctgtatagccgctgacatctaaaaggcagctcctgtcttgtaggaaagcttctttattagggtttggctctgctgttgcttccttccagagcatcttcctaatgctgttccccatgttgtagtgaccccccacacaccctaaaattattttcattgctacttcataactataattttgctactgttatgaagtgtaaatatctgtgttatccaatggtcttaggtgacccctgtgaaagggccacttgactccaaaagggccactgttctagacctatacaacggctcctccgttccttcattcctcagagaccagtttagcatgcctgccctgaactagagctaagagaaacacatatgaaggacgcccctcggggagctaacgctggatgcccacccactttctttcttcacacaggtgtctgaagcagct

SEQ ID NO: 7 - DNA, 3458 bp, Artificial Sequence
  3′ homologous arm
tatccacatctagcaaggaaccctgtctcaaaaattaaggtggaaaataattgaggaagacaactgattttgatctctggtctccacccctacacacatgtgaatgcacacacacacacacacacagagcacatatatacacaaagggattttcattacatatgattcatcaaagaaataagatcagctgaaactttaaagggagtaaggtttgatcctaaatcatttagttcacagacacctgttcaacaaacgctgagtgcctagtacagagatgagtactctgagagttaagaataagcttaggagatagagggatagaaaaatagatggatggatggatagatagatagatagatagatagatagatagacagacagacagatagatatagaaattaaggcaacatgaccaaatagagacaagatgaagtataaaaagagtagccaggaaagacctacagggtctggtaggagagatgacaaaccagggaggttgacatttgagccagatctaaaggaacaagagcaagttcgaaaaatggaggggaaaaaggtacatcaggtctaaggtcccaaaggaacaaagtcatggggatatgagagtgaatgatgcataaataaaatcaagaataaccagaagctaagtcaagacacaaaacaaattacagaaaaagaaaattgtactttatcccatataaagggagaattctctgagcttacaaagagctccttcaatacattagtggaagaacataatgaatgcccaacaaaacaataaacttaaagggttattaacagagcaagctggtcacagaaaggggaatgccatcagatcctgggcagacaagggttactcaacgttactcataagaaagtcaactcaaaaggactttgagatattgatgtgttcctatgtgacagacaaaaatatcataggtttaacacaccctgttaaagttggcaaaactgtagggaaaccccattgttaaacattttacatggaagtatgacctctacggaagaaaatgtgatgatggctttgtcccagcaattttacttctaaggctgtacactgtgtgtgtgtgtgtgtgtgtgtgtgtgtgtgtgtgtgtgtgtgtgtgtgtgtgtaacctataggaaagatagagaaccagaatcatttatttcacatggatcacttgcaaacattagaactaatgcaggaccagcaaaagactggttaaagactcaaagatgaaactgaagaatgagaaaagacctggctctttgcatcaatatggaactctctccaaaacattcagaaaaagcaagtgcgccacccagagaagcacactttgtgaggactaatggaaaagagcctgagctgttggagtccattccaaaggaattcatggcctaatgtacacaaagaaataaagtacccagactgcaaaagaaagaaagaaagaaagaaagaaagaaagaaagaaagaaagaaagaaagaaagaaagaaagaaagaaagaaaaaagaaagagagagagagagagagagagagagagaaagaaagagagagagggagagagagggagagagagggagagagagagagggagagagagagagagggagagagagagaggaagaaaggagagtggaaatacaaacttacataacagagggcagagaaaagggcaaaaccacctagtgtgctcatatttgatctgaaatttctagaggatacataaggaagagccaagaaataaaacatataagcacacataggaataaagccttccccctcccccaccgccgtatactttctatatagtttaatttttttgtgtgaatgtatcaccttctcagatataaaatagcaatgacgaaagctttctttccaaaacaaaagaacctaaatgaccaatcacaaaaaagcaaaggccgaacagtcagtgctgctgagcccccttccaacacctgctttctctccctccagctccctaagctaaggcagagtggggagggcagctctcacctctcctggttccaggacaccagtgcaaagcatgggaccactgttaggagaggcacccaaacagcacctgttccataacagagagctgccatgagtgatgcagaggtagccccatagcctaaggacccacccaccattccttggttcagtgacacaatccgtctaccccttcaggtccaatggcgacctgtaatggcatctttacttgtgaggtagccagattctaaatgcagatagatgtgcatggctctttagaggcaaaattgccagtgcttagctttgttaaatgcctgatggttcagaattccactcactatttgctgttgccccaaagagacaagttcaagacaagttcatagatgcactgtctgtggatgcttaacaaaagcggctggaagcaagatccagacaactcactggtaggaaagagcaaaccacacaaggacagagcaagggcattcacacacaaggaaaggcccaacccatgtgcagcgtctacccgtgtgcacctaaggcagagctggaagggcactgacccagctgaaagtggtcgttattgggggttgagacagacagagcaaggagaggtttcagggggaaccttaattttatcatttgactctgccagagagaatatattgtgtgtaacacacacttctttaatcctctgcattaaaatttgcaaatgccttccatttcaattcctggatctgagtcaatgactaaactgatcctccaaaagataattctattcctgatgtggagccaagttctctctcgatctctgtactaggtatccttggcctggaaggaagaaaggccttcctgctacatcaaatggctcagataacttgagaaaggaagagtcagaatgttccttgaactcttctcagaacacaaaatgtgggccaatagaaagtcaatttgtttgagactttggtaggtgacacatcaccccaccacagtttctaccagcttccaaggacagcccacgtgctggaactttggcctgatgtctgagatcctgtgacaagcaatccctcgggacaggcagtgggtgattttagaggacagggctgcagtaagcaaggaccaggctagggtagaacaagaatccagtgttgtctgggagcagagctggtctgagagacaggggagagggtggctgctaaggtctaagccagaggtggaggggaagtcatagtgtcttctatagacctctcttcacctttgttccagtcagggctttaggattgacctaactagaacatcaggaaatgaagaaataataggcatatacagaaaagctggggttgagtgagtcatgtatccttatgaagacatatcagcctgtcctaaaactcagaaagtttaatgtatacagtttaagtgggagccaggctagctagcaggtctctgtagggtagcatcctcaggatgct

SEQ ID NO: 8 - DNA, 1974 bp, Mus musculus
aagtctccagggcagagagggagtcaactcattggcgcttgagtcggcaaagaaatcaagatggccaaagttcctgacttgtttgaagacctaaagaactgttacagtgaaaacgaagactacagttctgccattgaccatctctctctgaatcagaaatccttctatgatgcaagctatggctcacttcatgagacttgcacagatcagtttgtatctctgagaacctctgaaacgtcaaagatgtccaacttcaccttcaaggagagccgggtgacagtatcagcaacgtcaagcaacgggaagattctgaagaagagacggctgagtttcagtgagaccttcactgaagatgacctgcagtccataacccatgatctggaagagaccatccaacccagatcagcaccttacacctaccagagtgatttgagatacaaactgatgaagctcgtcaggcagaagtttgtcatgaatgattccctcaaccaaactatatatcaggatgtggacaaacactatctcagcaccacttggttaaatgacctgcaacaggaagtaaaatttgacatgtatgcctactcgtcgggaggagacgactctaaatatcctgttactctaaaaatctcagattcacaactgttcgtgagcgctcaaggagaagaccagcccgtgttgctgaaggagttgccagaaacaccaaaactcatcacaggtagtgagaccgacctcattttcttctggaaaagtatcaactctaagaactacttcacatcagctgcttatccagagctgtttattgccaccaaagaacaaagtcgggtgcacctggcacggggactgccctctatgacagacttccagatatcataaaagcagccttatttcgggagtctattcacttgggaagtgctgacagtctgtatgtaccatgtacaggaaccttcctcaccctgagtcacttgcacagcatgtgctgagtctctgtaattctaaatgaatgtttaccctctttgtaagagaagagcaaaccctagtggagccaccccgacatatgatactatctgttattttaaagagtaccctatagtttgctcagtactaatcattttaattactattctgcatggcattcttaggaggatcaaaaagactctacacatattacagatgggttaacaaagggataaaacaactgaaaagcacactcaatgcatttggaatataaattcacagaccaatctcactgtgcaccttcggcttcaaaatgccagttgagtaggataaaggtataagaacttaatgctgtcattttcaaaaggaaggggacaatagctacatctttcctacctcagtgggttttactccagtgagatcatttggatgaaatcctcctgtaacagacctcaagaaggagacagactgttgaatgttatttttaagttattttatctatgtatttataaatatatttatgataattatattatttatggaacatccttaaatcctctgagcttgacggcaccctcgcagcagggttttctaggtggtcagttagatgtagtctcctctagagctccatgctacagacttttacactttttccacagccacgaagctctccgtacattcctgcacttgggagccctttcatcatgatcttaatctgtgctgtttactttgtgcatctaaaatgataattgagtcagtctttctccctcccgtccttaaagctgtctgggtattcttacatcattcagtctcacctgtaactaacaccaaccatctaaagatggaaagagcttaactgtgacaaccacatcactgatacctgaagtttcttttctagaatgtaatcagtgtttcccctggattccaatttttttttcaaaccacagtgtcatgtaactatcaacaataacaatcaactcattattattaatcataattaaataaaacaggtttgagctg

SEQ ID NO: 9 - protein, 270 aa, Mus musculus
MAKVPDLFEDLKNCYSENEDYSSAIDHLSLNQKSFYDASYGSLHETCTDQFVSLRTSETSKMSNFTFKESRVTVSATSSNGKILKKRRLSFSETFTEDDLQSITHDLEETIQPRSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 10 - DNA, 2017 bp, Homo sapiens
aagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaa

SEQ ID NO: 11 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 12 - DNA, 8704 bp, Artificial Sequence
  nucleotide sequence of 112775067-112783770 of NCBI accession number NC_000002.12
atggccaaagttccagacatgtttgaagacctgaagaactgttacaggtaaggaataagatttatctcttgtgatttaatgagggtttcaaggctcaccagaatccagctaggcataacagtggccagcatgggggcaggccggcagaggttgtagagatgtgtactagtcctgaagtcagagcaggttcagagaagacccagaaaaactaagcattcagcatgttaaactgagattacattggcagggagaccgccattttagaaaaattatttttgaggtctgctgagccctacatgaatatcagcatcaacttagacacagcctctgttgagatcacatgccctgatataagaatgggttttactggtccattctcaggaaaacttgatctcattcaggaacaggaaatggctccacagcaagctgggcatgtgaactcacatatgcaggcaaatctcactcagatgtagaagaaaggtaaatgaacacaaagataaaattacggaacatattaaactaacatgatgtttccattatctgtagtaaatactaacacaaactaggctgtcaaaattttgcctggatattttactaagtataaattatgaaatctgttttagtgaatacatgaaagtaatgtgtaacatataatctatttggttaaaataaaaaggaagtgcttcaaaacctttcttttctctaaaggagcttaacattcttccctgaacttcaattaaagctcttcaatttgttagccaagtccaatttttacagataaagcacaggtaaagctcaaagcctgtcttgatgactactaattccagattagtaagatatgaattactctacctatgtgtatgtgtagaagtccttaaatttcaaagatgacagtaatggccatgtgtatgtgtgtgacccacaactatcatggtcattaaagtacattggccagagaccacactgaaataacaacaattacattctcatcatcttattttgacagtgaaaatgaagaagacagttcctccattgatcatctgtctctgaatcaggtaagcaaatgactgtaattctcatgggactgctattcttacacagtggtttcttcatccaaagagaacagcaatgacttgaatcttcaatacttttgttttaccctcactagaggtccagagacctgtctttcattataagtgagaccagctgcctctctaaactaatagttgatgtgcattggcttctcccagaacagagcagaaccatcccaaatccctgagaactggagtctcctggggcaggcttcatcaggatgttagttatgccatcctgagaaaggccccgcaggccgcttcaccaggtgtctgtctcctaacgtgatgtgttgtggttgtcttctctgacaccagcatcagaggttagagaaagtctccaaacatgaagctgagagagaggaagcaagccagttgaaagtgagaagtctacagccactcatcaatctgtgttattgtgtttggagaccacaaatagacactataagtactgcctagtatgtcttcagtactggctttaaaagctgtccccaaaggagtatttctaaaatattttgagcattgttaagcagatttttaacctcctgagagggaactaattggaaagctaccactcactacaatcattgttaacctatttagttacaacatctcatttttgagcatgcaaataaatgaaaaagtcttcctaaaaaaatcatctttttatcctggaaggaggaaggaaggtgagacaaaagggagagagggagggaagcctaatgaaacaccagttacctaagaccagaatggagatcctcctcactacctctgttgaatacagcacctactgaaagaactttcattccctgaccatgaacagcctctcagcttctgttttccttcctcacagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaaggtaaggggtcaagcacaataatatctttcttttacagttttaagcaagtagggacagtagaatttaggggaaaattaaacgtggagtcagaataacaagaagacaaccaagcattagtctggtaactatacagaggaaaattaatttttatccttctccaggagggagaaatgagcagtggcctgaatcgagaatacttgctcacagccattatttcttagccatattgtaaaggtcgtgtgacttttagcctttcaggagaaagcagtaataagaccacttacgagctatgttcctctcatactaactatgcctccttggtcatgttacataatcttttcgtgattcagtttcctctactgtaaaatggagataatcagaatcccccactcattggactgctgtaaagattaagagtctcaggctttacagactgagctagctgggccctcctgactgttataaagattaaatgagtcaacatcccctaacttctggactagaataatgtctggtacaaagtaagcacccaataaatgttagctattactatcattattattattattttattttatttttttgagatggagtctggctctgtcacccaggctggagtgcagtggcgcaatctcggctcactgcaagctctgcctcctgggttcatgccattctcctgcctcagcctcctgagtagctgggaatacaggcacccgccactgttcccggctaattttttgtatttttagtagagacggagtttcaccgtggtctccatctcctcgtgatccacccaccttggcctcccaaagtgccgggattacaggcgtgagccaccgcgcccggcctatgattattattattattactactactactacctatatgaatactaccagcaatactaatttattaatgactggattatgtctaaacctcacaagaatcctaccttctcattttacataaaaggaaactaagctcattgagataggtaaactgcccaatggcatacatctgtaagtgggagagcctcaaatctaattcagttctacctgagtaaaaaaatcatggtttctcctccatccctttactgtacaagcctccacatgaactataaacccaatattcctgtttttaagataatacctaagcaataacgcatgttcacctagaaggttttaaaatgtaacaaaatataagaaaataaaaatcactcatatcgtcagtgagagtttactactgccagcactatggtatgtttccttaaaatctttgctatacacatacctacatgtgaacaaatatgtctaacatcaagaccacactatttacaactttatatccagcttttcttacttagcaatgtattgaggacattttagagtgcccgtttttcaccattataagcaatgcaacaatgaacatctgtataaataaatattcatttctctcaccctttatttccttagaatatattcctagaagtagaatttcccagagccatgaggatttgtgacgctattgatatgtgccactttgcactctctgtgacatatataattatttttaatgcattctttttttctcagagtgcattcatttgaaaacatagacgggaaatactggtagtcttccttgtcagttagaaacacccaaacaatgaaaaatgaaaaagttgcacaaatagtctctaaaaacaatgaaactattgcctgaggaattgaagtttaaaaagaagcacataagcaacaacaaggataatcctagaaaaccagttctgctgactgggtgatttcacttctctttgcttcctcatctggattggaatattcctaatatcccctccagaactattttccctgtttgtactagactgtgtatatcatctgtgtttgtacatagacattaatctgcacttgtgatcatggttttagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcaggtacattaaaatggcaccagacatttctgtcatcctcccctcctttcatttacttatttatttatttcaatctttctgcttgcaaaaaacatacctcttcagagttctgggttgcacaattcttccagaatagcttgaagcacagcacccccataaaaatcccaagccagggcagaaggttcaactaaatctggaagttccacaagagagaagtttcctatctttgagagtaaagggttgtgcacaaagctagctgatgtactacctctttggttctttcagacattcttaccctcaattttaaaactgaggaaactgtcagacatattaaatgatttactcagatttacccagaagccaatgaagaacaatcactctcctttaaaaagtctgttgatcaaactcacaagtaacaccaaactaggaagatctttattatctctgataacatatttgtgaggcaaaacctccaataagctacaaatatggcttaaaggatgaagtttagtgtccaaaaacttttatcacacacatccaattttcatggcggacatgttttagtttcaacagtatacatattttcaaaggtccagagaggcaattttgcaataaacaagcaagactttttctgattggatgcacttcagctaacatgctttcaactctacatttacaaattattttgtgttctatttttctacttaatattatttctgcaattttcccaatattgacatcgtgtatgtatttgccatttttaatatcactagacaattcaatcaggttgctacgttggtcccttgggtttactctaaatagcttgattgcaaatatctttgtatatattattgttttttctcctatcttgtaatttctttgagcacatcccaaagaggaatgcctagatcaatgggcacaaataatttgacagctcttattaaacattattctgtaagtaaaaactgaactacttttcagtatcactagcaacatatgagtgtatcagcttcctaaacccctccatgttaggtcattatgaacttatgatctaacaaattacagggtcttatcccactaatgaaattataagagattcaacacttattcagccccgaaggattcattcaacgtagaaaattctaagaacattaaccaagtatttacctgcctagtgagtgtggaagacattgtgaaggacacaaagatgtatagaattccattcctgacttccaggtatttacaccataggtggggacctaactacacacacacacacacacacacacacacacacacacacaccatgcacacacaatctacatcaacacttgattttatacaaatacaatgaatttactttctttttggttcttctcttcaccagtgaaatttgacatgggtgcttataagtcatcaaaggatgatgctaaaattaccgtgattctaagaatctcaaaaactcaattgtatgtgactgcccaagatgaagaccaaccagtgctgctgaaggtcagttgtcctttgtctccaacttaccttcatttacatctcatatgtttgtaaataagcccaataggcagacacctctaacaaggtgacactgtcctctttccttcctaccacagcccccacctgcccaccccactcccattgattccagaggcgtgcctaggcaggctctatgagaaaatataacagagagtaagaggaaaattaccttctttctttttcctttccctgcctgaccttattcacctcccatcccagagcatccatttattccattgatctttactgacatctattatctgacctacacaatactagacattaggacaatgtggcctgcctccaagaaactcaaataagccaactgagatcagagaggattaatcacctgccaatgggcacaaagcaacaagctgggagccaagtcccaaaatggggcctgctgcttccagttcccctctctctgcattgatgtcagcattatccttcgtcccagtcctgtctccactaccactttccccctcaaacacacacacacacaacagccttagatgttttctccactgataagtaggtgactcaatttgtaagtatataatccaagaccttctattcccaagtagaatttatgtgcctgcctgtgcttttctacctggatcaagtgatgtctacagagtagggcagtagcttcattcatgaactcattcaacaagcattattcactgagagccttctatttttcaggcatagtgccaacagcagtgtggacagtggtgcatcaaagcctctagtctcatagaacttagtcttctggaggatatggaaaacagacaacccaaacaaccaacaaaagagcaagatgctgcaaaaaaaaaaaaaaatgaatagggtgctaagatagagaaaagtgggagagtgctatttagacaaagtggtaaaaacaaagccccttgtgagatgagagctgccgacaggagggggcgggtcatggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcatggttgtgggtttttgggacattcagaggagtctgaatgcacccaggcctacaacttcaagatggtaaaggacagctccaaggatcagaagaagcattcttggaactggggcattttgagaaggaggaaaaatatgcagagactagtgcttgcagagcttgcatttggatttcatttgaggtacaatgaaaacccattaatgggtttcacacagtgcaatggcctgacctcacttatatttcctaaaatagaaaacagatcagaaggaaggcaatagagaagcagaaagtccaatgaggaggtttcacagcagtcatgggggtggggtaaggaaaagaagtggaaagaaacagacagaattgggttatattttggagatagaaccaacagaaggaagaggagaaacaacatttactgagaagggaaaaagtaggagaggaataggtttgggaaataaatcctgctgacattggaaaccccaaggaagcctcaaaagtatatttacttgctttagatttaaaagaataggaaagaagcatctcaacttggaatttgaaatctatttttccataaaagtattgttaaattctactcatactcacaagaaaagtacattctaaagagtatattgaaagagtttactgatatacttaggaattttgtgtgtatgtgtgtgtgtgtatgtgtgtgcgtgtgtttaaccttcaattgttgacttaaatactgagataaatgtcatctaaatgctaaattgatttccaaaggtatgatttgttcacttggagatcaaaatgtttagggggcttagaatcactgtagtgctcagatttgatgcaaaatgtcttaggcctatgttgaaggcaggacagaaacaatgtttccctcctacctgcctggatacagtaagatactagtgtcactgacaatcttcataactaatttagatctctctccaatcaactaaggaaatcaactcttattaatagactgggccacacatctactaggcatgtaataaatgcttgctgaatgaacaaatgaatgaagagcctatagcatcatgttacagccatagtcctaaagtgctgtttctcatgaaggccaaatgctaagggattgagcttcagtcctttttctaccatcttgttctctaacagaattctcttcttttcttcataggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtag

SEQ ID NO: 13 - DNA, 4800 bp, Artificial Sequence
  5′ homologous arm
tgtctcttcatgtttgtgcctctgaacacctctcccttgaggtagacaagtgaatagttaatttcaggatccctacgtcctcatgaatgtcaaactctgtaaacgctcacagccagaaagaacatagtcttgaattcacataaatgcatagcccctgtatgccttgaactcatttctacacccctgctgacacctaacagagtataaatgccctgtaagtagccattatactgtattgtctcctgaattcaacacagatgcttctcctccccatccccaagtacttctgccctcacttggttcagtctgcaaatgcagaacccagagacacaaagagccaaactttcccccagaaaattcagagttgtgcttctctcggcgttcaagtgattcctttcagatccgggtatcatgtgctgattttaaggttgattctgaaggcatttacaaatgtttctcaccttcatggtacagtgacaagctgtgccagagtccacagccatactccacttccaaacatcattcatcacaatggtggcctgccttctgttttcagggtatcacagaataaaaaagatttcactagacaaaggaaaggaaaaacctttctcatctttggggcggttgttgtttgtttgtttgtttgttgtggtaagtgcattgtttgagcacatgtgcatacaatttgtgtacctgtgagtaccgaggcccgaggttgctatcagagactgtcctcagtcactcctcttattgtctgaggcagcgtcttttaatgtaaccaagagcttgcagtttagttagtgtagctatcccagttgctccagattccctgggtctattttctgatactgccacggccacccagaatttatgtgggttataggtatctgaactccagtcctcttgctcctctggaagtccctttaacttctgagccaactcccctagccatcttttgttttttataaaatcgtagaattactcagagtaccttaaagtcgatgcatttcagcaaagacttgccagtgacagtgagaaagtattctaggattcagactataaatgagatctaatttcatcagaatctgagtttcaagttcaggctgtgtctgagtcttggtttctgtgaccttcacttggtcatttctttcttccaaggtcttcagctgcataaggacacaggaatggcacatgagtatgtgcagctggaggctgaactgagagcacactgttaaccttaaggcattgtcacatggaaagcatcactgagattcttagggcaagtgtgaccgtgcatggctgggtctataacccagcccccaggaggctgaagcaggaggagtctttgagagcagattcagacgcacaatgagaccctgtcaagaacaaaagacagatgctgggcagtggtggcgcacgcctttaatcccagcacttgggaggcagaggcagtcgaatttctgagttcgaagccagcctggtctacagagtgagttccaggacagccagggctacacagagaaaccctgtctcgaaaaccgaaaaaaagaaaaaagaaagaaagaaagaaagaaagaaagaaagaaagaaagaaagaaagaaagaaagaaagaaagaaagaaagaaagaaagaaagaaagaaagaaagaaaaaaagaaagagaacaaaagacaaagagaagagagaaggatgagatggagggaatgaagggaagtagagagagaaagaacaaggcagacagacagatcttttggagccataacatcagggcttacaaagcaaatccactcacctaccaacacatgtcattttgtcttgcaaaatgcttaattttgtggcttctgaaaggaaaattattaatgtttggcaaagcctatggtacagaatacaatatttatttctgaattatttaggaagcactgagccaaacacatcaggcccagtaaagccctgcccagtgctgtcctctctcccttccctaaaaggattggtttagatgctacagctctccctctctgaacctcctgcatcatcttttcccctcgaaaccactttccaagaaggccatatgaaagagtaggttgagagatacacagacccaaattggctgtttctcaaccaaactctcatttggggaattctctacagcacaggaatgaagatggagctggactggctgttgctaaagacagtagaaccaagggtgttttcccaaattgccaaagaggtgctgtgcactgttctcagaaaccaaatggctttggtgcctcggtagtgaccttcactcaaaaatgtcttccctagtgaaggtttcaaagccctgttttcctaagattcctattttctgctgtgaactaaggcatcatggtgatgactccacagactctccagtgactgaggccatcattagtgtcattatttttgtttagctgcatctccatagcagcccacattcacaatgcaccttgcaattgtttcttagtgattatcctcacaaggtgtccgacaggcagaagatgacaagattagcatttctgtgttgcctagagcagaaggcaggttaaggagggatgtggagctagctagctgctgtcactgctcatagatgtttagagacacccacccgagctttggctccagtctgcttctgtgcttggactacctttacgtaggatcactcatctttgagttttgtttgtaaatacagcatgaggagggacagactgggcagccacattggaattggagcgtttacacttgcagtagaagcaatgaacatctaaagagctccactccatgctttaaaagggctagagggactgggtcaaccaacacacataagctgctttacccctctcgccccaccgcttctggtttggtttgggttttatcccgtgtttcagtggtttgcttttctctctttgccattatttttctttccttgtctaccacccacttcctttatttaaaagttacatggcttaaatgccaactagaaaagctgaaacactgatttgctgagagtctggtgccctacctcttaagactggcctggcgtctgggaactgaaactatctctaataaaactgaccgggcagtgttgctccatgtaagaatccataccttcctctcccatagcctccactctggagctgaggcactatcatggctgggcctttgtccctgcctccaaaagtgagcagctgttctcactcagctctgatctacccagaggtacactagactttgtgtttaggtggtagggcaaggttactttgccataatgtggctgaggcagggaactctctcctgaagctgtcatctctgtaggaagtgtgtttatgttatccaaaggagtaagaggacccagatctttagggcaagtgtcacttttacctcctccacctttaaccataaggttattgtttcatactcagactgataatttattcaagcagattttcccctacaaagaggagggaagaagctcattttccctcaaatgccttagctgccaagtatcctgcccatccatattcagtcctggggcacaataaacaagttagcccaaaacacaaagtacagctatggagctccagtccttgtttggctttcactctgatttgaggatttcttccttcctcttccactggtgggcttcctgccgctcttcccgttttgtaagactacatgtctctaatcacatggattagcaagaaataggctgatcaagtcaacggcccgtggaatctttgcaagcaacattattaccaagtgtataatctgtccttctggccggtatcaggcagaatgaacatagaaagatactaagcagaattgtttcctgtaaattcccagtgacacacacatggccactcctacctgcttgaggaagactataaaaaggcagagaagcctgactcagacttaagtctccagggcagagagggagtcaactcattggcgcttgagtcggcaaaggtattgtccttatcacgtctaaggctatgaaaattgtcttttgtttgctccttgactgtcctctctcaaggcaccttgtgtaaggctatggttttttctcttcttcttcttcttcttcttcttcttcttcttcttcttcttcttcttcttcttcttctttctccttctccttctcctccttctccttctccttctcctcctccttctcttctcctcctcctcctcctcctcctcctcctcctcctcctcctcctcctcctcctcctcctcctcctcctcctccttctccttcttctaatttagtacaggggctgggatgagagatttaagaaaagaattctctttctagtgacagggaacttagggagcagctgaacaagattcatgggagggagtctaccagcaaagtcgaatctttttctggagattggacgtctctactggcaaaaatgagagaggccatcaggttcccattctcccttgcctatatataaggcagacaccccactctggtgattttaattttgtccttaatatattcacctattaaatgcctactatgtgtgatgcaatagactgaacacaaagaataccagggtagagaaaacattctaaaacaaaagtacaagttagtggaggagatagaagcatacagaaataatgtaagggagtacagagaagaatagaggaaattctgggcatgtgataagtcactcattgtaatttctcaagagcaaagtagggtcattgcccttgcaagcacggggtttagaagtggactgagctgacagccactggtgttctcttacagaaatcaag

SEQ ID NO: 14 - DNA, 3899 bp, Artificial Sequence
  3′ homologous arm
tgagaacaaggccttctaaaataactgagcaaaacccatatgcgttcacagagcctgaagcctgaagcagcagccgcagggcctacacaggtctgcccaggtttctgtacacacactctagctttcagtttatgctctgatgggactcctgaatggaaatgagtaagtctctgattcttctgcctactcttggagctctttcatttttctgttggcatagcttgtccaacttatatatgatgatttctgttttatcttactatattttgctacattttgttgttttccccaaaaaatgcatttaaaaaaaattcaggttccatagcttttctttctttttttttttttttgctagctgccagtattcacagatagatagcactgaaaattataatatttccaggctcagttaaaggatacaatccatattcagtacatgtgtatgccgatgtagatgtccatacaaactcaccatataatgtaccaccaaaaccaagtagtggttttagagacaatacagcaggttagaagctgctttcctgtgacctaatgaaaaagaaagtcagaatcaggagtaatagactctattttagaaagtggggttggaaattcacagaaacaataattgaacacctgaaatgtgcagtaacccttcacccaaattcttgtacatagccccgataagacgtgttgagctttggtggtatccaagcttttgtctgtctgcagtaagtagccatgtgttacatctcctcagggaaagtcttgtcacacaagtggatgtcgggatgtcagaccctatggagctagagttacaggcagatgtgttatgtaatatgggtgctgggaaccaactctggtggcctgccagagcactgcatgctccttaccccagccatctcagcgctttttatcatattaaaaaacaaaaacaaaagcaaaaacttttgtgattttttttttattctccttcactcacctgataatgtttaggtatgccagtgataaccttttccttctctttccttcatgattttttttttctttctagcaggcctttgttagcattttaactttaccttgtagatgtttttctgtttaaataaactattatgtttccatgttttatttccattcttaaatttatataattctgttctttacattttctattaggaaatctttgttattcttttaaaaactcttctaaccccttgcccttttctcttcccaccataacatccttctccaataaatatcttttacgagatttgctgcctcatatgactttcgcgttggaagaagccaagaagcaattgacagaggaagggaagaaacaaagaagtggagcggggcagaggctcctgagctcctgggctgagctggagataccctcccctgggaactgcactgcctcagctgacgaggcttcctctcaaagctgtgtggttccccggctctgatgtctcagggtgtccttccacaggaataccttctgaccctcagagctctgtggtttctgggtttccgagtctcagggtgcctttccaaggggacactgtcccacctcagagctgtgtggctcctgagctcctgtgtcttaggatgcccttcccttgagacacttttgacagattgcgccattttgacagctcccgcgagctgctcagggactacacggagttatgttgtttctgtccggtattcggctacaagaatgagaacaataactgatacggaccattttcgtttggcatgctattatttgcagccaaagccagcctaactgatagcacagagacttaggctccggcttgtttctccccgtttcttcaactgtcacaaccgtccttaacccaaatttggctacaattagagccactcctccgccagccagcatcacccaccagctcccacctcagaggcaccgctgcggaagtgacctagccatagggcgggccttctcagaaactctccaatcgcagagcctcaggggaggtccccagtgggaatcgccgaatgtccatttcctgttatcccgccctctccgtctttttattggcgaatttcaaagtcgcggaagccaataggagcgtccagacgggcgcccggttctcgtactagaacaaggcgcgccctttgaaggaagaaactgtcgccgtagagccatggtggggcccggccccactgccagcgcagccgcgggtaagtgtggtcggcagcctccagaagctgccaggcctcccccgcctgcttcagtctcccccgagctcagttaggttaaggacgctggacccgggccacggcgaggagcggcgcgctccgttgccgcagggggacacccctgcattgctagttatggccagaactttgacagcagtcaggtggcaggttcggccccagtccgagtctctgcaggttcactgataaaatgtgcgctgtctggaggttgattgataaactgtgggctgaggcttaaggatgctgtgaaccctggcctccgagggacctggagccctttctacctctccagtatgcccctcccccgtgtcacctttgagatgcacactctgtcctcacttatgggcctgcggttctgcgcaccgtcctaggcacacaatttgtaactcaagggttaaaaaaaaaagtcttgattccaaatcctggctttgctgcacattagtgggtaaatttaagcctgtcacttcatctgagtttcaaacagccaaatacctcataggattgatggttagtgtaaacagcttagtaaagagcttggcaccaggctgataccgttggtattgaaagcaagcataacaggactggtgactttatcaaagccaaaatagctacagggaagtatcatatttaaagatataaagggttttctgtaatgtttttggtgtttgctcgtgtccatgttttacagaagaacggtggcagaagctccaggagtacctggcagccaagggaaagctaaaggaccccaacgccaagtgagtttaccttcccatagttacactggctttgttgccccggttgctaacagcaatgaacggaccagcccttctgtttccagtctgtttgacttgcatcatggtgaatcctgtaataacaaaaggagcatttcagtccttgatttcaggcatgacttaaaaaacaaaacaaaacaaaaaaccagcaacccctaggcagtgctgtctattggcagaaatttaagcattttttttcctaacagtctccaaaggaaggaaggctctacactacactgttacccacatctctggaagctcttctggttccttctttctcctgggaaagttggttcccagcagcgcagattttgatcaagaaactggagggcatttctatggtgtgtgcttacgaagacaggagagttggttatggtcagtccacaacagtggcatgccagcgtcctcccaacttgtcctttggtgttccctgggcatgtatttgaggtggacagaacatcctttgcttttctcagcactgtaccatctttgccctgttatttctctctcttgcttagcagggtgtatttgcctcccacttgagtagctcggttggtgtgtgtatgccagagggggattttgggtgtcttgctctaacgctgtccaccttattcccataagaccggattgctcactgaacccgaagctcactgcttaggttaagcaggctggccagcactctctctcccatgatccactcatccctgtcccctagtgcctagggttagagggatactcaccgtgcctgatttaatgtggatgccggagatcttcctagcccaggtcattttaccttgggtttttctcacctctacttcctggattaaatgaaccaaaattgttttctcaacatacccataatgtctc

SEQ ID NO: 15 - DNA, 81 bp, Artificial Sequence
  connection between the upstream of the human DNA fragment in the “A fragment” and the mouse sequence
actttctttcttcacacaggtgtctgaagcagctatggcagaagtacctgagctcgccagtgaaatgatggcttattacag

SEQ ID NO: 16 - DNA, 61 bp, Artificial Sequence
  connection between the downstream of the human DNA fragment and the mouse sequence
ccatgcaatttgtgtcttcctaaagtatgggctggactgtttctaatgccttccccagggc

SEQ ID NO: 17 - DNA, 105 bp, Artificial Sequence
  connection between the upstream of the Neo cassette and the mouse sequence
tttggcaacaggaagatctctgggcttgacagcagccatctactagggttaacgaattccgaagttcctattctctagaaagtataggaacttcaggtctgaaga

SEQ ID NO: 18 - DNA, 170 bp, Artificial Sequence
  the downstream connection of the Neo cassette
cttcgaagttcctattctctagaaagtataggaacttcatcagtcaggtacataattaggtggatccaatattgatatcggtacctatccacatctagcaaggaaccctgtctcaaaaattaaggtggaaaataattgaggaagacaactgattttgatctctggtctcc

SEQ ID NO: 19 - DNA, 20 bp, Artificial Sequence
  primer
caggacatagcgttggctac

SEQ ID NO: 20 - DNA, 25 bp, Artificial Sequence
  primer
ttagccaacaggctacagaaccacg

SEQ ID NO: 21 - DNA, 25 bp, Artificial Sequence
  probe
catccataaccaaggctgccagtca

SEQ ID NO: 22 - DNA, 25 bp, Artificial Sequence
  probe
aattgctctgaccacttactgcccc

SEQ ID NO: 23 - DNA, 25 bp, Artificial Sequence
  probe
cttgttccttgctcttcaccagccc

SEQ ID NO: 24 - DNA, 26 bp, Artificial Sequence
  probe
cggccaatgcatcttctgtgtttcaa

SEQ ID NO: 25 - DNA, 21 bp, Artificial Sequence
  probe
ggatcggccattgaacaagat

SEQ ID NO: 26 - DNA, 22 bp, Artificial Sequence
  probe
cagaagaactcgtcaagaaggc

SEQ ID NO: 27 - DNA, 23 bp, Artificial Sequence
  primer
cttcctgggaaacaacagtggtc

SEQ ID NO: 28 - DNA, 25 bp, Artificial Sequence
  primer
gagagtgctgcctaatgtccccttg

SEQ ID NO: 29 - DNA, 25 bp, Artificial Sequence
  primer
gcccctggaaactaggtacttcaag

SEQ ID NO: 30 - DNA, 22 bp, Artificial Sequence
  primer
tgagcaagaggacctgaatgag

SEQ ID NO: 31 - DNA, 24 bp, Artificial Sequence
  primer
cattcacatgtgtgtaggggtgga

SEQ ID NO: 32 - DNA, 51 bp, Artificial Sequence
  connection between the upstream of the human DNA fragment in the “A fragment” and the mouse sequence
ggtgttctcttacagaaatcaagatggccaaagttccagacatgtttgaag

SEQ ID NO: 33 - DNA, 50 bp, Artificial Sequence
  connection between the downstream of the human DNA fragment and the mouse sequence
tactggaaaaccaggcgtagaagcagccttatttcgggagtctattcact

SEQ ID NO: 34 - DNA, 81 bp, Artificial Sequence
  connection between the upstream of the Neo cassette and the mouse sequence
ggagacaggagtcggggagacagaagggatggatatcgaattccgaagttcctattctctagaaagtataggaacttcagg

SEQ ID NO: 35 - DNA, 109 bp, Artificial Sequence
  the downstream connection of the Neo cassette
gaagttcctattctctagaaagtataggaacttcatcagtcaggtacataattaggtggatcccactatgtgtgagaacaaggccttctaaaataactgagcaaaaccc

SEQ ID NO: 36 - DNA, 20 bp, Artificial Sequence
  primer
gctcgactagagcttgcgga

SEQ ID NO: 37 - DNA, 24 bp, Artificial Sequence
  primer
gacttggacgagagaaggcgtgag

SEQ ID NO: 38 - DNA, 28 bp, Artificial Sequence
  probe
gaagtaaccctccagaaaagacttcccg

SEQ ID NO: 39 - DNA, 24 bp, Artificial Sequence
  probe
gcaacaccagctgtggtctctgat

SEQ ID NO: 40 - DNA, 27 bp, Artificial Sequence
  probe
ggctttcctgattcttctgtaccaagg

SEQ ID NO: 41 - DNA, 28 bp, Artificial Sequence
  probe
gacaggacctgactcttactggttgtat

SEQ ID NO: 42 - DNA, 25 bp, Artificial Sequence
  primer
tcgagagcgctgtttctcatgaagt

SEQ ID NO: 43 - DNA, 25 bp, Artificial Sequence
  primer
ggctccactagggtttgctcttctc

SEQ ID NO: 44 - DNA, 25 bp, Artificial Sequence
  primer
tcttcttctgggaaactcacggcac

SEQ ID NO: 45 - DNA, 25 bp, Artificial Sequence
  primer
gttagggacaactggtcagcactca

SEQ ID NO: 46 - DNA, 25 bp, Artificial Sequence
  primer
tccattcaggagtcccatcagagca

SEQ ID NO: 47 - DNA, 25 bp, Artificial Sequence
  primer
gacaagcgttagtaggcacatatac

SEQ ID NO: 48 - DNA, 24 bp, Artificial Sequence
  primer
gctccaatttcccacaacattagt

SEQ ID NO: 49 - protein, 268 aa, Rattus norvegicus
MATVPELNCEIAAFDSEENDLFFEADRPQKIKDCFQALDLGCPDESIQLQISQQHLDKSFRKAVSLIVAVEKLWQLPMSCPWSFQDEDPSTFFSFIFEEEPVLCDSWDDDDLLVCDVPIRQLHCRLRDEQQKCLVLSDPCELKALHLNGQNISQQVVFSMSFVQGETSNDKIPVALGLKGKNLYLSCVMKDGTPTLQLESVDPKQYPKKKMEKRFVFNKIEVKTKVEFESAQFPNWYISTSQAEHRPVFLGNSNGRDIVDFTMEPVSS

SEQ ID NO: 50 - protein, 270 aa, Rattus norvegicus
MAKVPDLFEDLKNCYSENEEYSSAIDHLSLNQKSFYDASYGSLHENCTDKFVSLRTSETSKMSTFTFKESRVVVSATSNKGKILKKRRLSFNQPFTEDDLEAIAHDLEETIQPRSAPHSFQNNLRYKLIRIVKQEFIMNDSLNQNIYVDMDRIHLKAASLNDLQLEVKFDMYAYSSGGDDSKYPVTLKVSNTQLFVSAQGEDKPVLLKEIPETPKLITGSETDLIFFWEKINSKNYFTSAAFPELLIATKEQSQVHLARGLPSMIDFQIS